SEQUENCE LISTING (40 sequences)

SEQ ID NO: 1 - protein, 509 aa, Mus musculus
MVDGVMILPVLMMMAFPSPSVEDEKPKVNQKLYMCVCEGLSCGNEDHCEGQQCFSSLSINDGFHVYQKGCFQVYEQGKMTCKTPPSPGQAVECCQGDWCNRNITAQLPTKGKSFPGTQNFHLEVGLIILSVVFAVCLLACILGVALRKFKRRNQERLNPRDVEYGTIEGLITTNVGDSTLAELLDHSCTSGSGSGLPFLVQRTVARQITLLECVGKGRYGEVWRGSWQGENVAVKIFSSRDEKSWFRETELYNTVMLRHENILGFIASDMTSRHSSTQLWLITHYHEMGSLYDYLQLTTLDTVSCLRIVLSIASGLAHLHIEIFGTQGKSAIAHRDLKSKNILVKKNGQCCIADLGLAVMHSQSTNQLDVGNNPRVGTKRYMAPEVLDETIQVDCFDSYKRVDIWAFGLVLWEVARRMVSNGIVEDYKPPFYDVVPNDPSFEDMRKVVCVDQQRPNIPNRWFSDPTLTSLAKLMKECWYQNPSARLTALRIKKTLTKIDNSLDKLKTDC

SEQ ID NO: 2 - DNA, 1530 bp, Mus musculus
  source 1..1530: mol_type = genomic DNA
atggtcgatggagtaatgatccttcctgtgctaatgatgatggctttcccttccccgagtgtggaagatgagaagcccaaggtcaaccagaaactttacatgtgtgtgtgtgagggcctctcctgcgggaacgaggaccactgtgaaggccagcagtgtttttcttctctgagcatcaacgatggcttccacgtctaccagaagggctgctttcaggtttatgagcaggggaagatgacgtgtaagaccccgccgtcacctggccaggctgtggagtgctgccaaggggactggtgtaacaggaacatcacggcccagctgcccactaaagggaagtccttccccggaacacagaatttccacctggaagttggccttatcatcctctcggtggtgtttgcagtatgtcttttagcttgcatccttggagttgctctcaggaagtttaagagacgcaatcaagagcgcctgaaccccagagacgtggagtatggtaccattgaagggctcatcaccaccaatgtgggagacagcactctagcggaactactagatcactcgtgtacatcaggaagtggctccggtcttcctttcctggtacagagaacggtggctcgccagataaccctgttggagtgtgtcgggaagggccggtatggagaagtatggaggggcagctggcaaggcgaaaatgtcgctgtgaagatcttctcctcccgagacgagaagtcatggttcagggagacggaattgtacaacactgtgatgttgaggcatgaaaatatcttaggtttcatcgcttcagacatgacctccagacactccagtacccagctgtggctcatcacacattaccatgaaatgggatcgttgtatgactaccttcagctcactactctggatacggttagctgccttcggattgtactgtccatagccagcggccttgcccatttgcacatagagatatttgggacccaagggaagtccgccattgcccatcgagatctgaagagcaaaaacatcctggtgaagaagaatggacagtgctgcatagcagatttgggcctggcagtcatgcattcccagagcacaaaccagcttgatgtgggaaacaacccccgtgtggggaccaagcgctacatggctccggaagtgctcgatgaaaccatccaagtggattgctttgattcttataagagggtcgatatttgggcctttggccttgttctgtgggaagtggccaggcgaatggtgagcaatggtatagtggaagattacaagccaccattctatgatgtggttcccaatgacccaagttttgaagatatgaggaaagttgtctgtgtggatcaacagaggccaaacatacctaacagatggttctcagacccgacattaacttctctggcgaagctgatgaaagagtgctggtatcagaacccatccgcaagactcacagctctacgtatcaaaaagactttgaccaaaatcgataattccctagacaaattaaaaactgactgttga

SEQ ID NO: 3 - protein, 509 aa, Homo sapiens
MVDGVMILPVLIMIALPSPSMEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLEVGLIILSVVFAVCLLACLLGVALRKFKRRNQERLNPRDVEYGTIEGLITTNVGDSTLADLLDHSCTSGSGSGLPFLVQRTVARQITLLECVGKGRYGEVWRGSWQGENVAVKIFSSRDEKSWFRETELYNTVMLRHENILGFIASDMTSRHSSTQLWLITHYHEMGSLYDYLQLTTLDTVSCLRIVLSIASGLAHLHIEIFGTQGKPAIAHRDLKSKNILVKKNGQCCIADLGLAVMHSQSTNQLDVGNNPRVGTKRYMAPEVLDETIQVDCFDSYKRVDIWAFGLVLWEVARRMVSNGIVEDYKPPFYDVVPNDPSFEDMRKVVCVDQQRPNIPNRWFSDPTLTSLAKLMKECWYQNPSARLTALRIKKTLTKIDNSLDKLKTDC

SEQ ID NO: 4 - DNA, 1530 bp, Homo sapiens
  source 1..1530: mol_type = genomic DNA
atggtagatggagtgatgattcttcctgtgcttatcatgattgctctcccctcccctagtatggaagatgagaagcccaaggtcaaccccaaactctacatgtgtgtgtgtgaaggtctctcctgcggtaatgaggaccactgtgaaggccagcagtgcttttcctcactgagcatcaacgatggcttccacgtctaccagaaaggctgcttccaggtttatgagcagggaaagatgacctgtaagaccccgccgtcccctggccaagccgtggagtgctgccaaggggactggtgtaacaggaacatcacggcccagctgcccactaaaggaaaatccttccctggaacacagaatttccacttggaggttggcctcattattctctctgtagtgttcgcagtatgtcttttagcctgcctgctgggagttgctctccgaaaatttaaaaggcgcaaccaagaacgcctcaatccccgagacgtggagtatggcactatcgaagggctcatcaccaccaatgttggagacagcactttagcagatttattggatcattcgtgtacatcaggaagtggctctggtcttccttttctggtacaaagaacagtggctcgccagattacactgttggagtgtgtcgggaaaggcaggtatggtgaggtgtggaggggcagctggcaaggggagaatgttgccgtgaagatcttctcctcccgtgatgagaagtcatggttcagggaaacggaattgtacaacactgtgatgctgaggcatgaaaatatcttaggtttcattgcttcagacatgacatcaagacactccagtacccagctgtggttaattacacattatcatgaaatgggatcgttgtacgactatcttcagcttactactctggatacagttagctgccttcgaatagtgctgtccatagctagtggtcttgcacatttgcacatagagatatttgggacccaagggaaaccagccattgcccatcgagatttaaagagcaaaaatattctggttaagaagaatggacagtgttgcatagcagatttgggcctggcagtcatgcattcccagagcaccaatcagcttgatgtggggaacaatccccgtgtgggcaccaagcgctacatggcccccgaagttctagatgaaaccatccaggtggattgtttcgattcttataaaagggtcgatatttgggcctttggacttgttttgtgggaagtggccaggcggatggtgagcaatggtatagtggaggattacaagccaccgttctacgatgtggttcccaatgacccaagttttgaagatatgaggaaggtagtctgtgtggatcaacaaaggccaaacatacccaacagatggttctcagacccgacattaacctctctggccaagctaatgaaagaatgctggtatcaaaatccatccgcaagactcacagcactgcgtatcaaaaagactttgaccaaaattgataattccctcgacaaattgaaaactgactgttga

SEQ ID NO: 5 - protein, 509 aa, synthetic construct
  REGION 1..509: note = synthetic polypeptide
MVDGVMILPVLMMMAFPSPSVEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLEVGLIILSVVFAVCLLACILGVALRKFKRRNQERLNPRDVEYGTIEGLITTNVGDSTLAELLDHSCTSGSGSGLPFLVQRTVARQITLLECVGKGRYGEVWRGSWQGENVAVKIFSSRDEKSWFRETELYNTVMLRHENILGFIASDMTSRHSSTQLWLITHYHEMGSLYDYLQLTTLDTVSCLRIVLSIASGLAHLHIEIFGTQGKPAIAHRDLKSKNILVKKNGQCCIADLGLAVMHSQSTNQLDVGNNPRVGTKRYMAPEVLDETIQVDCFDSYKRVDIWAFGLVLWEVARRMVSNGIVEDYKPPFYDVVPNDPSFEDMRKVVCVDQQRPNIPNRWFSDPTLTSLAKLMKECWYQNPSARLTALRIKKTLTKIDNSLDKLKTDC

SEQ ID NO: 6 - DNA, 1530 bp, synthetic construct
  misc_feature 1..1530: note = synthetic polynucleotide
  source 1..1530: mol_type = other DNA
atggtcgatggagtaatgatccttcctgtgctaatgatgatggctttcccttccccgagtgtggaagatgagaagcctaaggtgaaccccaaactctacatgtgcgtgtgcgaaggtctctcctgtggtaatgaggaccactgcgaaggccagcagtgtttttcttctctgagcatcaacgatggcttccacgtctaccagaagggctgctttcaggtttatgagcaggggaagatgacgtgtaagaccccgccgtcacctggccaggctgtggagtgctgccaaggggactggtgtaacaggaacatcacggcccagctgcccactaaagggaagtccttccccggaacacagaatttccacctggaagttggccttatcatcctctcggtggtgtttgcagtatgtcttttagcttgcatccttggagttgctctcaggaagtttaagagacgcaatcaagagcgcctgaaccccagagacgtggagtatggtaccattgaagggctcatcaccaccaatgtgggagacagcactctagcggaattattggatcattcgtgtacatcaggaagtggctctggtcttccttttctggtacaaagaacagtggctcgccagattacactgttggagtgtgtcgggaagggccggtatggagaagtatggaggggcagctggcaaggcgaaaatgtcgctgtgaagatcttctcctcccgagacgagaagtcatggttcagggagacggaattgtacaacactgtgatgttgaggcatgaaaatatcttaggtttcatcgcttcagacatgacctccagacactccagtacccagctgtggctcatcacacattaccatgaaatgggatcgttgtatgactaccttcagctcactactctggatacggttagctgccttcggattgtactgtccatagccagcggccttgcccatttgcacatagagatatttgggacccaaggcaagcccgccattgcccatcgagatctgaagagcaaaaacatcctggtgaagaagaatggacagtgctgcatagcagatttgggcctggcagtcatgcattcccagagcacaaaccagcttgatgtgggaaacaacccccgtgtggggaccaagcgctacatggctccggaagtgctcgatgaaaccatccaagtggattgctttgattcttataagagggtcgatatttgggcctttggccttgttctgtgggaagtggccaggcgaatggtgagcaatggtatagtggaagattacaagccaccattctatgatgtggttcccaatgacccaagttttgaagatatgaggaaagttgtctgtgtggatcaacagaggccaaacatacctaacagatggttctcagacccgacattaacttctctggcgaagctgatgaaagagtgctggtatcagaacccatccgcaagactcacagctctacgtatcaaaaagactttgaccaaaatcgataattccctagacaaattaaaaactgactgttga

SEQ ID NO: 7 - protein, 509 aa, synthetic construct
  REGION 1..509: note = synthetic polypeptide
MVDGVMILPVLMMMAFPSPSVEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLEVGLIILSVVFAVCLLACILGVALRKFKRRNQERLNPRDVEYGTIEGLITTNVGDSTLAELLDHSCTSGSGSGLPFLVQRTVAHQITLLECVGKGRYGEVWRGSWQGENVAVKIFSSRDEKSWFRETELYNTVMLRHENILGFIASDMTSRHSSTQLWLITHYHEMGSLYDYLQLTTLDTVSCLRIVLSIASGLAHLHIEIFGTQGKPAIAHRDLKSKNILVKKNGQCCIADLGLAVMHSQSTNQLDVGNNPRVGTKRYMAPEVLDETIQVDCFDSYKRVDIWAFGLVLWEVARRMVSNGIVEDYKPPFYDVVPNDPSFEDMRKVVCVDQQRPNIPNRWFSDPTLTSLAKLMKECWYQNPSARLTALRIKKTLTKIDNSLDKLKTDC

SEQ ID NO: 8 - DNA, 1530 bp, synthetic construct
  misc_feature 1..1530: note = synthetic polynucleotide
  source 1..1530: mol_type = other DNA
atggtcgatggagtaatgatccttcctgtgctaatgatgatggctttcccttccccgagtgtggaagatgagaagcctaaggtgaaccccaaactctacatgtgcgtgtgcgaaggtctctcctgtggtaatgaggaccactgcgaaggccagcagtgtttttcttctctgagcatcaacgatggcttccacgtctaccagaagggctgctttcaggtttatgagcaggggaagatgacgtgtaagaccccgccgtcacctggccaggctgtggagtgctgccaaggggactggtgtaacaggaacatcacggcccagctgcccactaaagggaagtccttccccggaacacagaatttccacctggaagttggccttatcatcctctcggtggtgtttgcagtatgtcttttagcttgcatccttggagttgctctcaggaagtttaagagacgcaatcaagagcgcctgaaccccagagacgtggagtatggtaccattgaagggctcatcaccaccaatgtgggagacagcactctagcggaactactagatcactcgtgtacatcaggaagtggctccggtcttcctttcctggtacagagaacggtggctcaccagataaccctgttggagtgtgtcgggaagggccggtatggagaagtatggaggggcagctggcaaggcgaaaatgtcgctgtgaagatcttctcctcccgagacgagaagtcatggttcagggagacggaattgtacaacactgtgatgttgaggcatgaaaatatcttaggtttcatcgcttcagacatgacctccagacactccagtacccagctgtggctcatcacacattaccatgaaatgggatcgttgtatgactaccttcagctcactactctggatacggttagctgccttcggattgtactgtccatagccagcggccttgcccatttgcacatagagatatttgggacccaaggcaagcccgccattgcccatcgagatctgaagagcaaaaacatcctggtgaagaagaatggacagtgctgcatagcagatttgggcctggcagtcatgcattcccagagcacaaaccagcttgatgtgggaaacaacccccgtgtggggaccaagcgctacatggctccggaagtgctcgatgaaaccatccaagtggattgctttgattcttataagagggtcgatatttgggcctttggccttgttctgtgggaagtggccaggcgaatggtgagcaatggtatagtggaagattacaagccaccattctatgatgtggttcccaatgacccaagttttgaagatatgaggaaagttgtctgtgtggatcaacagaggccaaacatacctaacagatggttctcagacccgacattaacttctctggcgaagctgatgaaagagtgctggtatcagaacccatccgcaagactcacagctctacgtatcaaaaagactttgaccaaaatcgataattccctagacaaattaaaaactgactgttga

SEQ ID NO: 9 - DNA, 25214 bp, synthetic construct
  misc_feature 1..25214: note = synthetic polynucleotide representing the 8431 Allele
  source 1..25214: mol_type = other DNA
aggggcgaagctcatagaactgaattgtttctcctcctcacctctcctttgcagatgagaagcctaaggtgaaccccaaactctacatgtgcgtgtgcgaaggtctctcctgtggtaatgaggaccactgcgaaggccagcagtgtttttcttctctgagcatcaacgatggcttccacgtctaccagaagggctgctttcaggtttatgagcaggggaagatgacgtgtaagaccccgccgtcacctggccaggctgtggagtgctgccaaggggactggtgtaacaggaacatcacggcccagctgcccactaaaggttcccatggctccttctattactagatcaaggggttttgtgagggttgccccaagtctgtgtgggttaaggatctgcgtagccagaagcattcttccgacttgctgagccagagctgctgggccatggaaggtcactctgggtgaggaagacaaaggcatggcctttgggattgaaaaggagagtgatgttttggggaaagtgagtgatgtgacatgcatccagaggaagggtgaagctcgagtaactttaaataatgccaattatttaaagttagccagtagcagcacccacgtccaccttctgtctagtaatgtccaacacctccctcagtccaaacactgctctgcatccatgtggctcccatttatacctgaagcacttgatggggcctcaatgttttactagagcccacccccctgcaactctgagaccctctggatttgtctgtcagtgcctcactggggcgttggataatttcttaaaaggtcaagttccctcagcagcattctctgagcagtctgaagatgtgtgcttttcacagttcaaatccatgtggctgtttcacccacctgcctggccttgggttatctatcaggacctagcctagaagcaggtgtgtggcacttaacacctaagctgagtgactaactgaacactcaagtggatgccatctttgtcacttcttgactgtgacacaagcaactcctgatgccaaagccctgcccacccctctcatgcccatatttggacatggtacaggtcctcactggccatggtctgtgaggtcctggtcctctttgacttcataattcctaggggccactagtatctataagaggaagagggtgctggctcccaggccacagcccacaaaattccacctgctcacaggttggctggctcgacccaggtggtgtcccctgctctgagccagctcccggccaagccagcaccgccaccatgggaacccccaagaagaagaggaaggtgtccgagctgatcattagcggatcttcgggtggcttcttaaggaatatcggaaaggagtaccaagaagccgctgaaaactttatgcgatttatgaacgatcagggagcttacgcccctaatacgctccgggatctgcggctcgtgttccattcatgggctaggtggtgccacgccaggcagctcgcgtggtttcctatctcgcccgagatggcccgggaatacttcctccaattacatgatgctgacttggcctctaccaccatcgacaagcattacgcaatgctgaacatgctgctctcgcattgtggtctgcctccgctgtctgacgataagtctgtgtcccttgccatgcgacgaatcagacgggaggcagctaccgaaaagggggagcgaactgggcaggtaaatataaaatttttaagtgtataatgatgttaaactactgattctaattgtttgtgtattttaggccataccactgcgctgggacgatctcaagctattggacgtgcttctttcaagatccgaaagactggttgatctgcgcaaccgggcattcctgtttgttgcctacaacacgctgatgaggatgagcgagatctcaaggatcagagtgggcgatctggaccagaccggcgataccgtaaccctgcacatctcgcacacaaaaaccatcacaacagcggcaggactggacaaggtgctttctagaagaactactgctgttttgaacgattggctggacgtgtctggccttagggagcatcccgatgcggtgctgtttcctcccatccacagaagtaacaaagctaggatcaccactaccccccttacagctcctgctatggaaaagatcttctcagacgcgtgggtgttgctgaataagcgagatgctaccccaaataaaggtcggtacaggacctggaccggccacagtgcccgagttggggcagctatcgatatggccgagaaacaggtgtctatggtggagatcatgcaagaaggtacgtggaagaaacctgaaaccctgatgcggtatttgcggagaggcggtgtatctgtgggtgcaaattcacggctgatggattcctgaaccggtgtaatgatcataatcagccatatcacatctgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccatgcatggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtgttgacaattaatcatcggcatagtatatcggcatagtataatacgacaaggtgaggaactaaaccatgaaaaagcctgaactcaccgcgacgtctgtcgagaagtttctgatcgaaaagttcgacagcgtgtccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgattgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagacctcgacctgcagccgctaggtaactttaaataatgccaattatttaaagttatgtacataactataacggtcctaaggtagcgagctagcggcatgtgtgtttcgagagaagtaggaacactagcttggtgtctgtgtgaagtgcctttcccttgttaagaagaagatgaaggaagccttgcccacccttccaaatcagatccctagccttctgaaacagaagtcagatgagggtgtgtatgagccctgaagttatgccatttcccctcatgtatttcttatggattctgaaccctttggaagtcggtcttcctctgaaactctttttctcatatggtgaactctcattgtcttatctcacattactgtcaatattaatataggctcaccccaagccaaatttattcttgtaatgtacagtacctcttaataggtttaattatatgattttgtccttgtggaagagggaaagcacaaataggataagagaggacacacacagccttcattgctaggactgtcacaaggcaggaaggaggcaagaggctggaaaccaaaagaccaataggtcacatggcagttttgaagttactatgaccccctttgggtagatagaacaggctacattgggatataaaaataggatcaaacacaagggtttgaaaaaggattggctatttgagtaattaactcacaggtcatttccttatgctcactgagttaatcctttgctaaaagaagggatggacaggaagaaacatctgtattcacacaaaaaccttgccagcagtgtgaagatcctcacgaacacattatgctgcacactacgcaacacctttacttatgtgttgacttggttgtgtttgtgtgtgcgaaagcgcaggcacacatggccctgcatttgcacatgtgtgttgagcatgtcagaggacaacttgtaggaattggttctcttattgtgtgggttccagggatcaaactcaggctgtcaggctttgcagcaactcctgagaccctctgagccctttcactctcccaacacccagtgttttcacagaagacatttcctggaaggaatgtcctatttgagaggccacaatatctgtcacttattaaatcacttttacgaataattatacagatggtatgcactatagagcaccggcctgattctcaggacttggggagttaaagaaacaatataaaggaacctttaagtagagagtggatggctgcagggaactctgagacaggtcaagcttagggaggagatcagcgctttctcactgggatattacagtatagaggctgttcatcagggctcgttgaaaagcatctttgcatctgaagtccacgttcataagcgttaacatcaaggattatgtgtctcttatatttaagaagctcttaagcattcatgcattaaaatacagttgattcttggtggaatcctttctaaaccccagttctatttaattgatcacttaaatgagtgtctgggccattgtctaaatgttaggaagtattttagttgtgaattgttttgatacgtgagatggtttggtcgggatcaggagccattacctaagatattggtaaataacaaagaaaactgtggtctccacgcctacttactcttttcttcccattctccagggaagtccttccccggaacacagaatttccacctggaagttggccttatcatcctctcggtggtgtttgcagtatgtcttttagcttgcatccttggagttgctctcaggaagtttaagagacgcaatcaagagcgcctgaaccccagagacgtggagtatggtaccattgaagggctcatcaccaccaatgtgggagacagcactctagcggtgagtttgtgaccccagagggaggcctacgaataacgagatggaggtggacactttcttgttttgaatgtgcaaagccaacaatgactatgcagacatgtatggtgactagtcactgggctaaaagccaaggacatccgtttcagacaggcacttaagggatggggctaggagattttagtagatccttggtggtgacttttctttggagaatgtgaggaaaagggacaaagaatagttggtggaccagagattcaaagaacttgacgtggtggtcttcagttactctatttgtaacaacaaacattttttttaaaggaagcatcttttcattattattaagaaatccttttccctttagattatgtgcatccgccataatttagagacttgacatctccagaagtagccattaagattttgatgctccagtactgcccccttgtggcgagtagtcggctggttgaaccagggtcctctagtacaactgggcatagcacatacagagtttaaaatgcttgagtagccagcttaacaagtaaatagggggaaaagcaaaccgtttataccttttatcatttcaacatacaataaaattgttgatatatttgataagttttcaaagtcagttacctggtgctaggaaccccccccccccccccccgagtttacttcctcagcatagaagatgctattatttatacatttggaaatttcttcctggcccgtgaggcggctccgagggtaaagatgctcactgctgagcctaatgacctaaatttaatccccaggactgatagggtaggaggagaaaaccaactcccacaagctgtcctctgacttccaaatatgtaccgtggcaaggctttccccctacccccaacaaataagcaaacaaatatgtgcaataaaaatttaaaattactcctgcattttaaagctatagcacgatactgtaagtgttcctttagttactcttctaaacttttagttaactcggggaagtaggtcactcaccataagcagcctactatagagtgcagaagaataaagtaacagccttctccatggatactacatggtttctctgcaaaagacaaagctctgtgggtgttaggtgcgaacaacacaaccaaatcagctctttccttgtcacaatctagctagtcgcaagtcaatcaagttatctttgtaatgtgattcagagctgtttaatgctttgctccctcctcatcccagatcgtcatgcccgcaggctggcttcactacctgacaagtccgtaagtgcctgggaattctgtagctaggcttcctctacaactgccttttgttgcttttctggaaaagatgtacgctaagcagtggtcatctttggctcacatgctagacctgggcagccataggtaccccctgtagtcaggtgtcagtacgttaataatgctggatccaggctgggaactgtcttgccctgattcctgtcccatcagggttctgtttgaatgaaagaggaacaagttagcctgtgtacatcgttttgcataccaatttgagttgtgatgtggtggaactgggagaaagggtgtaccttgtttccatgaaatgtttgtccagaagtctggtttaaggtgtaggggaaaggaggtggggtttatgagatttttgtagcagaaagacatgacgatgattcccgacaggcaaagcaagatgctaatgtagattagtcagaactgtgtttctccgagctgctagcatgtctaattaccaagggcagtaattggaatatctagtgtaaattactgtttgaatagcttttaggattttgaaatgggaatgaaagcctgcgccataacagtcttctcaacacagcccagccacgcagctgacagagtgtcaccatataatgacacagtaaggaataacgagatgtccttatttgcttatttaaacaatatgttcatttatgtgagctgacaaagagtaaccttgctgaagctcgcccacggctgggccttcagaacttgaggtaggtaggtctttattagtctttgcttgattgcctgatgccccatctcccaaatctgcacacttaattagaaccaaaactatgcctcatggaaaagcaacagctcaatgtgcagagggttttttttttttttttccctcaaaaataattgaacgaatagagtcaccaagcagggcaagagggaattgttggggagacacgaagcctatttcatcacattttttttcttcttgattatccttttgtgacttttaggggttctgaatttcttatgttgagtgtttagcttttggtaatcttactgatccacagtatctcacggtcttgctccacagaaaggagagaagatgaaattcacagaaatctatgtgttgctttaatgggccagtttatgtgttgggaaaggaagaactttgggctgggttaatctttccaccatcaccatggctataaggctgtctgtgccttagagatagtccaggccgggtcctgccgctgggcctttgctgagagtgtgaaagaaaacttcatgaatgaactaactggcagccttttgccagtatgctcatttgggaaatcaatgtgcgactctggtgttatgttttaagtgaaatatatatatattttttttcctgtttcctttctaatttgcagttcatatggaaaaatgataagtaggtgttaatttagtgacctgcttgttcatatattacctgaaaaaaatgagaatcttcatccacagaaacacccacgcacacacctataagtaccaatttaatgacccacatattattagaatcttttcattacagatattacacttggctcttcatagtcctaatttgcttgcttccaattagacacctttagtttgtaaggcaaccgaagtagatcacttgatagacatttagtggacatagcagaatagataatagtagacaatgcccagctcttggtgctcaagtatcccccacccccattcttcctactgactcctcacgcacagccccgtagcctttctaatgtgcgcgttagcttagctctgtttccttccttgcatatttgattagaaaactctatagagcttaagaaagaatgacttatccatcaggaaaacctgctgtaggtttcgcttctaagtttcccaagacatataactagtcctttcaaaggagattttcaggcatttgaatgggtcacatgtccagagttgaccggtataacttcgtataatgtatgctatacgaagttatgctgagcggattgctgcccttcatgtgagttacaatgtcatgcatgctaatactccaaagtgggagctatattgctcaatcgtttcttttcccccttgtcttaaaccacaggaattattggatcattcgtgtacatcaggaagtggctctggtcttccttttctggtacaaagaacagtggctcgccagattacactgttggagtgtgtcggtaattcttttttttcctttctttgtgggtaatatgcaatgttagttttgtttttgaagtaaaagatggaacttggaaaatctgctttttgttacctgttgtttccttattaaacttatacgcgtataacttcgtataaggtatcctatacgaagttatcacctcctgcatttaatgaggaagcaataataagcaacaagaagcagacttttccaagccctggctgacttcgagaatcacaaccaacattgcctgctgcccaaaaggaagggaaacatagaattaccgacacactccaacagggttatctggtgagccaccgttctctgtaccaggaaaggaagaccggagccacttcctgatgtacacgagtgatctagtagttcctgtgatgtaaaacaaggagaaaaagaaatcactgagcaacagtgcccgcttcagcgtcctgcaatacatgctgtgggaacccacaggatttccaggtaccataacttcgtatagcatacattatacgaagttatccggacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggccgcgactgcgtacgataacttcgtataggataccttatacgaagttatggatccccaagcaaccatgtggttattccacttcagggggtgtgagacagccatgggggtctctgttctgtgtgactcacggttgagagcttgctgaagttgtggtaagagagtcatccctgactagaacagacaatgcctagcgtgaatccacgaggctgcgttgaacaggctcgagaagttcctattccgaagttcctattctctagaaagtataggaacttctatgcatggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgtacctcaggtccggccggaataaaagatctttattttcattagatctgtgtgttggttttttgtgtgaattctggccttgggggagggggaggccagaatgactggccttgggggagggggaggccagaatgagtcgactggccttgggggagggggaggccagaatgactggccttgggggagggggaggccagaatgaaccggaataaaagatctttattttcattagatctgtgtgttggttttttgtgtgaattctggccttgggggagggggaggccagaatgactggccttgggggagggggaggccagaatgagtcgactggccttgggggagggggaggccagaatgactggccttgggggagggggaggccagaatgaaccggcgatatcgaagttcctattccgaagttcctattctctagaaagtataggaacttcgctagctcaagttactgtgtatcaattagacacaagagcttatgaaaatatacatttaagaactcgagtgaaccttactatgtttttttttttaattaagtgcttagtacgaagacctatatacattttgatagcctttcatttttctccctatgacatagtgtaggtcagcatattaattgtgaaaaaaattgtactcctttatccgttaaaatttctaaaaaaaagaaaaaaatctgctgttgctgcatgtgggtatatgacgtatgtgtgtgggtgtgtgcacgccacagcacacgtggactttggaggacagtgctgtagagttggttctcacaccgtacctttatatgggctctgggggttgaactcatatcatcaggctttgcggcaagcgcttcacccattgaaccacctcagaggcccaccttccttcattgttcagaatgcacacacctcgtctggtgttgggaattatcagaagtactttaaaaagcggtgcaacggtcagttcggctagcaggttagcagagatgcgtgaaaaggatttggcacacattctttggtcttctgaattggcagaatgaattccaggaattgtgaatttttgggtcaggcatgatttaagaataataaaatgatgatggtgacggcggtggcggcttcatgaattccatgaggctggtacagaatgctcttctagcttttgagacagggtctcttctggcccaggcagacccaaaactcggtatctagccgagaatcgtcttttgatgctctctcctctctctcttgagtactgggggttattataagtatacatccctatggccaaatagagttttaaagtcctgattttcagggctgaggggatggctcagataataagtgcttgttgcacaagcatgaagactggattttgatccccagtgcctatgtagaaaagcttgcacagtggcatgtatgtatgtgcctatagtctcagtgctggggatgcagaaagggcaagatctgtggagtttgctggccaagcagtctaggtttatctgtgagccttaggtttagtctgaaacctgtctcaattaagtaagtgactgagccaaacacccagtattagcgtcacgtatatatacgtagctgccctccccttcccattccccccacacatgaaggacacactttattttacacttgatcttagccaaaaggccgagaagcgatcacactttattttaaatacaatcctcagtttcactatttgtatgtggagtgtagatatttgcaggtagcatgacgggttttgtcagcttctcattgagatggtgtgtaggaatgccttgtattgtctcaccccttctggtgagcaggctttttgggtggctatagcagctcctgccttctgacagtctgtttggcggacgctgtcgcaggctatttgatcacattgtgaatcagaaaaaccctgtggtggtgtggctcagccctagcacacacctttaatccaaagagctttcttcgaatggaattataaagtcagccacaggtcaagaggcagggcaagcaaccagttgacagggagtgaacataggaaaacccaggaaagagaaaggcgggtctttgagttgaggggtatctaactatatcatggaggagaaagagccttttttcatctgggacatggtgaactaggaaggtcagccgggtgctttctctgcctttcggagctagcaggctttcaccacagcatctggctttggagtcttcattcgggataccaaatgtttgggattttgttttaaaaacaacaggaagcagctgaccattatgaactggtccctgcgctttctgccatatgaaggaggaacagtgtcaaatctagtagtaagatctgcagtgggcagcacggcttgcctgtgacacacaggggccagcaaaggagctgtggtaccttttccccaggtctgcacctactgttcctatttcccattctctgcagctccaccacaactgactaagactagaaagagatggagaaacaaaacaaacaaaacaacaacaacaaaacagaggcactcagcacccacaaccagaatggttgtcttgacgtctaactggcatcctgtttgtgtatgaacaaggatacctgggtagtgatggcaggcagcagaggggccatgcacctgctgctcctgtcttcttgccccaacaacccatcactcacagatagtcccacatacctcagagactggctccagagtcttgctcctctgaactgtctacagtgtcgcactcgatagatgctcttcaacatttttcaccactgtaaatttaactattgcattttgggggttgacattagagcctggtgggtggagagcctgttcagaagttttttgtttttgtgtccaaggtgtagcttggcgggaaaagatggatagatgatcactttagttcaagaacgtggggatctgaccatgtcaacccttccagccctatgcatcctctttgtgggactttctggccagggacttttggggaactgtaccactcttttagtggctttgacataagattacattagcaaaaagcatttgacagccattaaaatagatttctgttctgatttaatccacctaccatgtcgtatattttcttttcttaacttaaaaaaaaaaaaaagatttacaacaacaaacaactttgtatccacaattctttgggatgtttttcttgccttctcagtgaaacagtgactccccagagaaaatcaaaaagttgcaagatggaacaaaggcaggaaaagcttagcatttatttcctgcttttgtagcttctggcttggtggcatcttcaggcagttagtcctcatcttgaacagaaaggttgagccactcctacgacttgattaaaatgacttgtaaaccaagtagactttgtgtgattattgacagtagctcacaaggaactctggcctctcttagcatccctttatattagtcattgctattggatagggttatcagttcttgtcatggaagctcagtttgctggatatccgtgctgccacctctggagctggatggtacccagtgcctcacacctgtgaggcaggcactctatcgacaagtcgcatcccctgcccttggagcttttgtgacagtttcaggtatgtcactctggcacacctgctttctttctctgtgtagggtcttattggcttagttttccagcagaagattgtacttgtgtgcaggcaggtggcaccacagctgcctattacagccccgtgatggtgcagtcaccacatgcctgggcagaaagcctcattgcactgtagtttcggttcccgctgcacgcctgtcatccaagccgactacacctcatgttgacccccactggttttgagataatgacatcctcttccctatctctttgatcatctctacctataattaaagagatgactggaagggggacatgcccttgataagcatgtggctactacatagaggtggggggaggggcatgaatcagacaggtcagatcccagctgaagttaccctattttgtaaactatgctcttcaagttactaaaatgaaatcctgaattatattaagtgtataggggttttgcctgcatgtatgtctgggtatcccaagtatgcctcgtgtttgaggaggtaagaagagggcattgcattccctggtactgatgtaataatagaagggtgtgtgctgcgtgggttcttggaatcaaatctgggtcgtccggaagagcctccaagactcttaacccctgagccatttcttcagccctcgggtgacttaagaaatgttttatctaccagttttgtgtgtgtatgtttatggcgcacatatgtgactgtgctttgtggagtcagttctctattttcacttttatatagattctagagtccgaacccaggatcacacttggcaagctcatttgcttgctggtactgtctttctaactgccaaaggctctttattttagtgtagtctgagtgtgacatcactggggcacatctaaaccaacaccctgaccagtgtcagaagttagagctcttctggaattatgtctatgactttgaaactctgcttttgaaatatatctttctccaagaaaccccttggaggtggtggttcacaaccttctaccagcactggggactgtcattaatctccaggatgtcaaactgcccctctgtggggcaagaggagcttaaatatattcaggcatttgggctgagaaatgcttctcagggtccagtggaaagggggggaaccagagaaaccatctagttatttagatatatacccctaccccttgggcttttgatgctacattgaaatttagtgatcagtagtatgtgctaaattgtactacctttcatgtgttttgaaaaactagatgccaaaagtttctagccttttgacattgtgactttccactgtcatctacaaacaggttgagctgcctgcatagttagtctggggcacaagtcagacaccctcaggaaagcaacttaaatataaatgtgaagttagttatcaggtactgtactgactggaaggcagattcctgccatgattcaactggtagggaaagataggttttaccttatttttgtacatctttataggtgaagtatttgcagttctagactgagtttgtctagcaaataatgaagagtttggggcatcaggctttgtttactactaggagtttcttggtcaggctgtgtgtcctgtctttacccttctatacaacaaagaataaattcagtgtcacacaaagaaacacagaaccaatgcaaagttattggcatggcagtttcttttcaaaaaggatctaccagaacccaaatccagctagtgaactcatgtgccaaatggcctccatattttatttttgatacaggtggtgactttgttctatgccatgggtgggagcttggcttcacagtctgacataattggttagtcagaagagaagagcaggaggttcaaatataaggacactgtcgggttcactgtgtttgagaggaaaaggaaagccaacccatttcttacatatatcatctgttttgctgtccacagggaagggccggtatggagaagtatggaggggcagctggcaaggcgaaaatgtcgctgtgaagatcttctcctcccgagacgagaagtcatggttcagggagacggaattgtacaacactgtgatgttgaggcatgaaaatatcttaggtgagtaccaggtgagctttcaccagctggtctccatagagataagggccggccctttctctctcccatttgccaaatctgaggtgtgtggagtctcttctggaaaacaacatagtaaccctcagcatgtatagactacttaattttttgacaggtatttttgtttgtttgtttaaatctgcacataggttatgaattgcctgcatatgaaaaggaaatccacagcaggacctttctcctcctgtatctgtggatgtattgaagcaaagccctgctctgctccgttgttcacttcaaggctccttgggtggttctgcttgccccacccttcctgaatatttattactggaatcaaactaagaaaggtctttggattctttcctagagattcacagctcccagggacgcgtaattagatgcacatttcctcaaactaggcatcatttttaaagaccagatttacagagggattcagcatacattttaatttgaaagctcctcgccttacttctgggactcacaaagtatagatgaattttcatcatcttatcactaaagcttagcaatgcttaagaaatattcgaggtgcctactacaatcctgctatggcaatgagagggtttccatagtttcaaaaggctttgagtgtatttccattgatacacacatatggtgacagctgcctagggtagatcttgagaagtagaaatgaaatccatgtagaatttcactgtacaccatagtgttggccactgtgtggctattggattatgtctgggcatgggacaattcacttctatcccggtctatcactttagcatttaaatgtgacccaagttgttggtgacagatgattggatggttgtccaaagctttggtttaacagtatggtggcgatgttcactctcaggtacatttaaaagtgattttgagaggcaggagagatagcttagcggttaagaacactggctgtgcttgcagaggtcctgagttctactcccagcacttgacatggcattagtttgtaactcttgttcctgggaatctgatgacctcttctggcttctgtgggcactgcatccacatggcacacatacatagtaaataaataaataaatatgcaaataaataaacaagtcaataccgcatgccacatacatgtaagtaaaacacacagacacacacacacacacacacacacacacacacacacacacacaccaaaccaatcaatcaatcaatcaatcaatcaatcaataatacatgtctctgctccccgattagcctgcaatgttggtgacttctcggcctgacattcatgactccctagttgtcgatttcttttgaacttcacttattaagtgtctctgattttggctgcaaccagtatttttctttttgcgggactgtgacaagcaggtggctttgattgcttgcatcactcttgattgttcactatctcctttctgttctctctctcatctttgctctatcaaaacgtctctgtcagtttctttgaagagactcctctgtttgtctatacccctcatggcctatgctgccttcgggggtctgtgtgtggtagttgtgatggcagcaccggcataggacgtgctggatagttcctgtgccgtgagttcttctgtaaagtctccattgcagccttcaagaggaggtattcttatttccccactttcactaacagaatcaccaatgagaggcgaaatactagagccccagctcatgctgcaaggactaacccttcaccatagttccctgccatgcactgcagtctacgggatagccatatcactttgcactggagtcatgtcttagattgcaggtagggtaggaggtgtggacatttatctatagtcaaacttgccacgagccttccaatcttccagcttcctccggagcaaactggaattgagcaagagttggttatgttagagcctaggatctataacaaaggcttatctttaaatagagtcactaataatgggtagatgcacatactcctaagaggcatgtgaagtcttgaagccgtttgaaggaagagccttctgcacctgacgatcaaagttacatctttctgagcactcactgccttatcatgttaaccctggaggctgactagcatggagatctactcccaggtcttttattgcactcctttaaaaagatttatgatatcatcttatgcattgtttttgagacagggcctactgtatctcaagctagcctcaaattcactagctaaggaggagcttgaatttctgatccccctgctttacctctcaagtgcaggaagtacctccgtgcctggtttatgtggagctgagattaaacccaggatttgttgtgtgcaaggcaagccctgctccaacttcatccccaggccaagtttatgtattttaagcacaaagttgaatagagttaaatttggcatgggtgcggactaatcgtcatccaacctccataactgtcttgtgctcttgtgtcttacgtgatgggctggggagatgctcagtgcttgctgtcttttaactcctgggatcctgtggtaggggagaacagacttctacatgttaatctttgacttccatcatgcgtcattggtacatgtgcacatacacacaaaataaataccagtaaatgtaataataaatataaataaaattgaaaattgaaagcactactaaggggctgagtgatgtctcagtaggtaaagtggttattttgcagggctgacatttgaatccccagaacccacataaagctatgctgtagtgcaggcatgcccctacttggagactggagatggagacaggagaattgctagaagcccataggccagctagttttgtatacacaacagtgaacagcaagagaaaccatgcctcaaacaaggtgaaaagcaaggatggacacctgagattgttctgtgacctgcacacatggaccctcttacatgtaagaggcctcacatcatggatacacacatacatgtgcatgcataagcacacacacaagagtactgtagactgtttcatgtcactgtttcttgagatgactagggaggggaacaaactatcgctggaaatacagaattgcaaatgttcttcctatacaccgtgtgctcagcaccatacacattaagcattctgaatgcactggcttcctgtccatatccctttaggatgaatgctgtcactctcatctcatctcatagactagaaagcaagatggagatttctggtgccttgatcttgatcacccagtccaaggctggctgagccactatgtaaacagtggtacagctttgggttcaaccacctcaacgcctcacattccagcgtctctgctggtttctgagggtgttgccactgaaaacttcaggtttcagatggctttttgttcttgaagagtttgggacaatggctctgcagaaatatttggtctaaccatccttccttctgttgcaaatacatccagtgggtacgtctgagggcccccccccccacctccaggcttttattaaccatggactgcttaggactccagacacaaacatcccagtgctcccaggctgggaagcaaatgtacttacctaaatttgaggagtaaacagttgaaaaaaaaatcctgctcatttaaaagtcctagttcctggaatatgcgcgcacacacacacatgagtcctttttcctggtttagaccataaatgaatggtatgaacaacatccttctttaaggtttaagggtctaggaggaagggagaaagggagagttcgggaggggaggagggaggggaagttccgattgggatgtaaagtaaataaataaatgtattaataaaaaataaacaaaagtaattaagagtatgtttaaatgtcaaatataacattattcatgatgaaggtgaagttcatcagagattcatttgtttcctcattcatataaaataacaagatcaaatagagtgagaccagaaaaaaaaaaagatctaagagtcatagggaaggtgagagcaaaagagcttgcttattatatatgattattgttttctcaggggaagaaataggtgctggctactgcacagttatatgtcagcagtgtgctccctttggaatggcactctagtttggcactttattttgagggaagctaatgcagactgagtcccattgtttgtttgtttgtttgtttgtttgtttgtttgttttgagacagggtttctctgtgtagccctggctgtcctggaactcactttgtagaccaggctggcctcgaacccagaaatattgcctctgcctcccaagtgctgggattaaaggtgtgcgccaccacgcccggcaagaatcccattcttagactgtgtacgaaagtacctttatgtctttccgcatctgttagtgtcccacacctcttccggacaaattcacttgtcaccactggacgcaagcttattccaatgtctaatctaaattattaaaattctttattttagaaaaaaacatattgagagcgtacagatggtgcagcaggcacggcagtaacaatgtctccctgctctgggccacttggattctgggtgttaggcccatggggcgtctctcatgcgccctctgcagtgctcacttcctgttgagattctccagcttcgggttgagctcttccagcttacaagcacgatccgtacagttcttattatctgaatgattaagaacaccacaaatatggataacacaagactgtttctgtttgcttgtcttgtttgtttttttaaaaacttaatgaatcaatcatctcataggtttacctctgttttatgctggaataaatataagccaagtagaccatggcaattagccctattacttaaaatctacagccaggttaggactagatgacaagcatctaggtttcaggtctggtagggttttagggtgcgatgtgtagtgtttgcatagcgaagaggtggaaatttatttatttatttattcattcatttatttatttatttatttattttgttttttgagacagggtttctctatatagccctggctgtcctggaactcactttgtagaccaggctggcctcaaactcagaaatccacctgcctctgcctcgaaagagtataaatgccgcttacagtggaaactggagggtcatgtgctgttcgttggccctgcacctttcaggagtctacactgtcgctgttctttgttgcctttgtaactgttaaatatttcttgttctacattagatctgctccgagatgatttggagtcctcagagttacatggcggagttgtcactgaaaaattctgtcctcctccctctccaggtttcatcgcttcagacatgacctccagacactccagtacccagctgtggctcatcacacattaccatgaaatgggatcgttgtatgactaccttcagctcactactctggatacggttagctgccttcggattgtactgtccatagccagcggccttgcccatttgcacatagagatatttgggacccaaggcaagcccgccattgcccatcgagatctgaagagcaaaaacatcctggtgaagaagaatggacagtgctgcatagc

SEQ ID NO: 10 - DNA, 17768 bp, synthetic construct
  misc_feature 1..17768: note = synthetic polynucleotide representing the 8432 Allele
  source 1..17768: mol_type = other DNA
aggggcgaagctcatagaactgaattgtttctcctcctcacctctcctttgcagatgagaagcctaaggtgaaccccaaactctacatgtgcgtgtgcgaaggtctctcctgtggtaatgaggaccactgcgaaggccagcagtgtttttcttctctgagcatcaacgatggcttccacgtctaccagaagggctgctttcaggtttatgagcaggggaagatgacgtgtaagaccccgccgtcacctggccaggctgtggagtgctgccaaggggactggtgtaacaggaacatcacggcccagctgcccactaaaggttcccatggctccttctattactagatcaaggggttttgtgagggttgccccaagtctgtgtgggttaaggatctgcgtagccagaagcattcttccgacttgctgagccagagctgctgggccatggaaggtcactctgggtgaggaagacaaaggcatggcctttgggattgaaaaggagagtgatgttttggggaaagtgagtgatgtgacatgcatccagaggaagggtgaagctcgagtaactttaaataatgccaattatttaaagttatgtacataactataacggtcctaaggtagcgagctagcggcatgtgtgtttcgagagaagtaggaacactagcttggtgtctgtgtgaagtgcctttcccttgttaagaagaagatgaaggaagccttgcccacccttccaaatcagatccctagccttctgaaacagaagtcagatgagggtgtgtatgagccctgaagttatgccatttcccctcatgtatttcttatggattctgaaccctttggaagtcggtcttcctctgaaactctttttctcatatggtgaactctcattgtcttatctcacattactgtcaatattaatataggctcaccccaagccaaatttattcttgtaatgtacagtacctcttaataggtttaattatatgattttgtccttgtggaagagggaaagcacaaataggataagagaggacacacacagccttcattgctaggactgtcacaaggcaggaaggaggcaagaggctggaaaccaaaagaccaataggtcacatggcagttttgaagttactatgaccccctttgggtagatagaacaggctacattgggatataaaaataggatcaaacacaagggtttgaaaaaggattggctatttgagtaattaactcacaggtcatttccttatgctcactgagttaatcctttgctaaaagaagggatggacaggaagaaacatctgtattcacacaaaaaccttgccagcagtgtgaagatcctcacgaacacattatgctgcacactacgcaacacctttacttatgtgttgacttggttgtgtttgtgtgtgcgaaagcgcaggcacacatggccctgcatttgcacatgtgtgttgagcatgtcagaggacaacttgtaggaattggttctcttattgtgtgggttccagggatcaaactcaggctgtcaggctttgcagcaactcctgagaccctctgagccctttcactctcccaacacccagtgttttcacagaagacatttcctggaaggaatgtcctatttgagaggccacaatatctgtcacttattaaatcacttttacgaataattatacagatggtatgcactatagagcaccggcctgattctcaggacttggggagttaaagaaacaatataaaggaacctttaagtagagagtggatggctgcagggaactctgagacaggtcaagcttagggaggagatcagcgctttctcactgggatattacagtatagaggctgttcatcagggctcgttgaaaagcatctttgcatctgaagtccacgttcataagcgttaacatcaaggattatgtgtctcttatatttaagaagctcttaagcattcatgcattaaaatacagttgattcttggtggaatcctttctaaaccccagttctatttaattgatcacttaaatgagtgtctgggccattgtctaaatgttaggaagtattttagttgtgaattgttttgatacgtgagatggtttggtcgggatcaggagccattacctaagatattggtaaataacaaagaaaactgtggtctccacgcctacttactcttttcttcccattctccagggaagtccttccccggaacacagaatttccacctggaagttggccttatcatcctctcggtggtgtttgcagtatgtcttttagcttgcatccttggagttgctctcaggaagtttaagagacgcaatcaagagcgcctgaaccccagagacgtggagtatggtaccattgaagggctcatcaccaccaatgtgggagacagcactctagcggtgagtttgtgaccccagagggaggcctacgaataacgagatggaggtggacactttcttgttttgaatgtgcaaagccaacaatgactatgcagacatgtatggtgactagtcactgggctaaaagccaaggacatccgtttcagacaggcacttaagggatggggctaggagattttagtagatccttggtggtgacttttctttggagaatgtgaggaaaagggacaaagaatagttggtggaccagagattcaaagaacttgacgtggtggtcttcagttactctatttgtaacaacaaacattttttttaaaggaagcatcttttcattattattaagaaatccttttccctttagattatgtgcatccgccataatttagagacttgacatctccagaagtagccattaagattttgatgctccagtactgcccccttgtggcgagtagtcggctggttgaaccagggtcctctagtacaactgggcatagcacatacagagtttaaaatgcttgagtagccagcttaacaagtaaatagggggaaaagcaaaccgtttataccttttatcatttcaacatacaataaaattgttgatatatttgataagttttcaaagtcagttacctggtgctaggaaccccccccccccccccccgagtttacttcctcagcatagaagatgctattatttatacatttggaaatttcttcctggcccgtgaggcggctccgagggtaaagatgctcactgctgagcctaatgacctaaatttaatccccaggactgatagggtaggaggagaaaaccaactcccacaagctgtcctctgacttccaaatatgtaccgtggcaaggctttccccctacccccaacaaataagcaaacaaatatgtgcaataaaaatttaaaattactcctgcattttaaagctatagcacgatactgtaagtgttcctttagttactcttctaaacttttagttaactcggggaagtaggtcactcaccataagcagcctactatagagtgcagaagaataaagtaacagccttctccatggatactacatggtttctctgcaaaagacaaagctctgtgggtgttaggtgcgaacaacacaaccaaatcagctctttccttgtcacaatctagctagtcgcaagtcaatcaagttatctttgtaatgtgattcagagctgtttaatgctttgctccctcctcatcccagatcgtcatgcccgcaggctggcttcactacctgacaagtccgtaagtgcctgggaattctgtagctaggcttcctctacaactgccttttgttgcttttctggaaaagatgtacgctaagcagtggtcatctttggctcacatgctagacctgggcagccataggtaccccctgtagtcaggtgtcagtacgttaataatgctggatccaggctgggaactgtcttgccctgattcctgtcccatcagggttctgtttgaatgaaagaggaacaagttagcctgtgtacatcgttttgcataccaatttgagttgtgatgtggtggaactgggagaaagggtgtaccttgtttccatgaaatgtttgtccagaagtctggtttaaggtgtaggggaaaggaggtggggtttatgagatttttgtagcagaaagacatgacgatgattcccgacaggcaaagcaagatgctaatgtagattagtcagaactgtgtttctccgagctgctagcatgtctaattaccaagggcagtaattggaatatctagtgtaaattactgtttgaatagcttttaggattttgaaatgggaatgaaagcctgcgccataacagtcttctcaacacagcccagccacgcagctgacagagtgtcaccatataatgacacagtaaggaataacgagatgtccttatttgcttatttaaacaatatgttcatttatgtgagctgacaaagagtaaccttgctgaagctcgcccacggctgggccttcagaacttgaggtaggtaggtctttattagtctttgcttgattgcctgatgccccatctcccaaatctgcacacttaattagaaccaaaactatgcctcatggaaaagcaacagctcaatgtgcagagggttttttttttttttttccctcaaaaataattgaacgaatagagtcaccaagcagggcaagagggaattgttggggagacacgaagcctatttcatcacattttttttcttcttgattatccttttgtgacttttaggggttctgaatttcttatgttgagtgtttagcttttggtaatcttactgatccacagtatctcacggtcttgctccacagaaaggagagaagatgaaattcacagaaatctatgtgttgctttaatgggccagtttatgtgttgggaaaggaagaactttgggctgggttaatctttccaccatcaccatggctataaggctgtctgtgccttagagatagtccaggccgggtcctgccgctgggcctttgctgagagtgtgaaagaaaacttcatgaatgaactaactggcagccttttgccagtatgctcatttgggaaatcaatgtgcgactctggtgttatgttttaagtgaaatatatatatattttttttcctgtttcctttctaatttgcagttcatatggaaaaatgataagtaggtgttaatttagtgacctgcttgttcatatattacctgaaaaaaatgagaatcttcatccacagaaacacccacgcacacacctataagtaccaatttaatgacccacatattattagaatcttttcattacagatattacacttggctcttcatagtcctaatttgcttgcttccaattagacacctttagtttgtaaggcaaccgaagtagatcacttgatagacatttagtggacatagcagaatagataatagtagacaatgcccagctcttggtgctcaagtatcccccacccccattcttcctactgactcctcacgcacagccccgtagcctttctaatgtgcgcgttagcttagctctgtttccttccttgcatatttgattagaaaactctatagagcttaagaaagaatgacttatccatcaggaaaacctgctgtaggtttcgcttctaagtttcccaagacatataactagtcctttcaaaggagattttcaggcatttgaatgggtcacatgtccagagttgaccggtataacttcgtataatgtatgctatacgaagttatgctgagcggattgctgcccttcatgtgagttacaatgtcatgcatgctaatactccaaagtgggagctatattgctcaatcgtttcttttcccccttgtcttaaaccacaggaattattggatcattcgtgtacatcaggaagtggctctggtcttccttttctggtacaaagaacagtggctcgccagattacactgttggagtgtgtcggtaattcttttttttcctttctttgtgggtaatatgcaatgttagttttgtttttgaagtaaaagatggaacttggaaaatctgctttttgttacctgttgtttccttattaaacttatacgcgtataacttcgtataaggtatcctatacgaagttatcacctcctgcatttaatgaggaagcaataataagcaacaagaagcagacttttccaagccctggctgacttcgagaatcacaaccaacattgcctgctgcccaaaaggaagggaaacatagaattaccgacacactccaacagggttatctggtgagccaccgttctctgtaccaggaaaggaagaccggagccacttcctgatgtacacgagtgatctagtagttcctgtgatgtaaaacaaggagaaaaagaaatcactgagcaacagtgcccgcttcagcgtcctgcaatacatgctgtgggaacccacaggatttccaggtaccataacttcgtatagcatacattatacgaagttatccggacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggccgcgactgcgtacgataacttcgtataggataccttatacgaagttatggatccccaagcaaccatgtggttattccacttcagggggtgtgagacagccatgggggtctctgttctgtgtgactcacggttgagagcttgctgaagttgtggtaagagagtcatccctgactagaacagacaatgcctagcgtgaatccacgaggctgcgttgaacaggctcgagaagttcctattccgaagttcctattctctagaaagtataggaacttcgctagctcaagttactgtgtatcaattagacacaagagcttatgaaaatatacatttaagaactcgagtgaaccttactatgtttttttttttaattaagtgcttagtacgaagacctatatacattttgatagcctttcatttttctccctatgacatagtgtaggtcagcatattaattgtgaaaaaaattgtactcctttatccgttaaaatttctaaaaaaaagaaaaaaatctgctgttgctgcatgtgggtatatgacgtatgtgtgtgggtgtgtgcacgccacagcacacgtggactttggaggacagtgctgtagagttggttctcacaccgtacctttatatgggctctgggggttgaactcatatcatcaggctttgcggcaagcgcttcacccattgaaccacctcagaggcccaccttccttcattgttcagaatgcacacacctcgtctggtgttgggaattatcagaagtactttaaaaagcggtgcaacggtcagttcggctagcaggttagcagagatgcgtgaaaaggatttggcacacattctttggtcttctgaattggcagaatgaattccaggaattgtgaatttttgggtcaggcatgatttaagaataataaaatgatgatggtgacggcggtggcggcttcatgaattccatgaggctggtacagaatgctcttctagcttttgagacagggtctcttctggcccaggcagacccaaaactcggtatctagccgagaatcgtcttttgatgctctctcctctctctcttgagtactgggggttattataagtatacatccctatggccaaatagagttttaaagtcctgattttcagggctgaggggatggctcagataataagtgcttgttgcacaagcatgaagactggattttgatccccagtgcctatgtagaaaagcttgcacagtggcatgtatgtatgtgcctatagtctcagtgctggggatgcagaaagggcaagatctgtggagtttgctggccaagcagtctaggtttatctgtgagccttaggtttagtctgaaacctgtctcaattaagtaagtgactgagccaaacacccagtattagcgtcacgtatatatacgtagctgccctccccttcccattccccccacacatgaaggacacactttattttacacttgatcttagccaaaaggccgagaagcgatcacactttattttaaatacaatcctcagtttcactatttgtatgtggagtgtagatatttgcaggtagcatgacgggttttgtcagcttctcattgagatggtgtgtaggaatgccttgtattgtctcaccccttctggtgagcaggctttttgggtggctatagcagctcctgccttctgacagtctgtttggcggacgctgtcgcaggctatttgatcacattgtgaatcagaaaaaccctgtggtggtgtggctcagccctagcacacacctttaatccaaagagctttcttcgaatggaattataaagtcagccacaggtcaagaggcagggcaagcaaccagttgacagggagtgaacataggaaaacccaggaaagagaaaggcgggtctttgagttgaggggtatctaactatatcatggaggagaaagagccttttttcatctgggacatggtgaactaggaaggtcagccgggtgctttctctgcctttcggagctagcaggctttcaccacagcatctggctttggagtcttcattcgggataccaaatgtttgggattttgttttaaaaacaacaggaagcagctgaccattatgaactggtccctgcgctttctgccatatgaaggaggaacagtgtcaaatctagtagtaagatctgcagtgggcagcacggcttgcctgtgacacacaggggccagcaaaggagctgtggtaccttttccccaggtctgcacctactgttcctatttcccattctctgcagctccaccacaactgactaagactagaaagagatggagaaacaaaacaaacaaaacaacaacaacaaaacagaggcactcagcacccacaaccagaatggttgtcttgacgtctaactggcatcctgtttgtgtatgaacaaggatacctgggtagtgatggcaggcagcagaggggccatgcacctgctgctcctgtcttcttgccccaacaacccatcactcacagatagtcccacatacctcagagactggctccagagtcttgctcctctgaactgtctacagtgtcgcactcgatagatgctcttcaacatttttcaccactgtaaatttaactattgcattttgggggttgacattagagcctggtgggtggagagcctgttcagaagttttttgtttttgtgtccaaggtgtagcttggcgggaaaagatggatagatgatcactttagttcaagaacgtggggatctgaccatgtcaacccttccagccctatgcatcctctttgtgggactttctggccagggacttttggggaactgtaccactcttttagtggctttgacataagattacattagcaaaaagcatttgacagccattaaaatagatttctgttctgatttaatccacctaccatgtcgtatattttcttttcttaacttaaaaaaaaaaaaaagatttacaacaacaaacaactttgtatccacaattctttgggatgtttttcttgccttctcagtgaaacagtgactccccagagaaaatcaaaaagttgcaagatggaacaaaggcaggaaaagcttagcatttatttcctgcttttgtagcttctggcttggtggcatcttcaggcagttagtcctcatcttgaacagaaaggttgagccactcctacgacttgattaaaatgacttgtaaaccaagtagactttgtgtgattattgacagtagctcacaaggaactctggcctctcttagcatccctttatattagtcattgctattggatagggttatcagttcttgtcatggaagctcagtttgctggatatccgtgctgccacctctggagctggatggtacccagtgcctcacacctgtgaggcaggcactctatcgacaagtcgcatcccctgcccttggagcttttgtgacagtttcaggtatgtcactctggcacacctgctttctttctctgtgtagggtcttattggcttagttttccagcagaagattgtacttgtgtgcaggcaggtggcaccacagctgcctattacagccccgtgatggtgcagtcaccacatgcctgggcagaaagcctcattgcactgtagtttcggttcccgctgcacgcctgtcatccaagccgactacacctcatgttgacccccactggttttgagataatgacatcctcttccctatctctttgatcatctctacctataattaaagagatgactggaagggggacatgcccttgataagcatgtggctactacatagaggtggggggaggggcatgaatcagacaggtcagatcccagctgaagttaccctattttgtaaactatgctcttcaagttactaaaatgaaatcctgaattatattaagtgtataggggttttgcctgcatgtatgtctgggtatcccaagtatgcctcgtgtttgaggaggtaagaagagggcattgcattccctggtactgatgtaataatagaagggtgtgtgctgcgtgggttcttggaatcaaatctgggtcgtccggaagagcctccaagactcttaacccctgagccatttcttcagccctcgggtgacttaagaaatgttttatctaccagttttgtgtgtgtatgtttatggcgcacatatgtgactgtgctttgtggagtcagttctctattttcacttttatatagattctagagtccgaacccaggatcacacttggcaagctcatttgcttgctggtactgtctttctaactgccaaaggctctttattttagtgtagtctgagtgtgacatcactggggcacatctaaaccaacaccctgaccagtgtcagaagttagagctcttctggaattatgtctatgactttgaaactctgcttttgaaatatatctttctccaagaaaccccttggaggtggtggttcacaaccttctaccagcactggggactgtcattaatctccaggatgtcaaactgcccctctgtggggcaagaggagcttaaatatattcaggcatttgggctgagaaatgcttctcagggtccagtggaaagggggggaaccagagaaaccatctagttatttagatatatacccctaccccttgggcttttgatgctacattgaaatttagtgatcagtagtatgtgctaaattgtactacctttcatgtgttttgaaaaactagatgccaaaagtttctagccttttgacattgtgactttccactgtcatctacaaacaggttgagctgcctgcatagttagtctggggcacaagtcagacaccctcaggaaagcaacttaaatataaatgtgaagttagttatcaggtactgtactgactggaaggcagattcctgccatgattcaactggtagggaaagataggttttaccttatttttgtacatctttataggtgaagtatttgcagttctagactgagtttgtctagcaaataatgaagagtttggggcatcaggctttgtttactactaggagtttcttggtcaggctgtgtgtcctgtctttacccttctatacaacaaagaataaattcagtgtcacacaaagaaacacagaaccaatgcaaagttattggcatggcagtttcttttcaaaaaggatctaccagaacccaaatccagctagtgaactcatgtgccaaatggcctccatattttatttttgatacaggtggtgactttgttctatgccatgggtgggagcttggcttcacagtctgacataattggttagtcagaagagaagagcaggaggttcaaatataaggacactgtcgggttcactgtgtttgagaggaaaaggaaagccaacccatttcttacatatatcatctgttttgctgtccacagggaagggccggtatggagaagtatggaggggcagctggcaaggcgaaaatgtcgctgtgaagatcttctcctcccgagacgagaagtcatggttcagggagacggaattgtacaacactgtgatgttgaggcatgaaaatatcttaggtgagtaccaggtgagctttcaccagctggtctccatagagataagggccggccctttctctctcccatttgccaaatctgaggtgtgtggagtctcttctggaaaacaacatagtaaccctcagcatgtatagactacttaattttttgacaggtatttttgtttgtttgtttaaatctgcacataggttatgaattgcctgcatatgaaaaggaaatccacagcaggacctttctcctcctgtatctgtggatgtattgaagcaaagccctgctctgctccgttgttcacttcaaggctccttgggtggttctgcttgccccacccttcctgaatatttattactggaatcaaactaagaaaggtctttggattctttcctagagattcacagctcccagggacgcgtaattagatgcacatttcctcaaactaggcatcatttttaaagaccagatttacagagggattcagcatacattttaatttgaaagctcctcgccttacttctgggactcacaaagtatagatgaattttcatcatcttatcactaaagcttagcaatgcttaagaaatattcgaggtgcctactacaatcctgctatggcaatgagagggtttccatagtttcaaaaggctttgagtgtatttccattgatacacacatatggtgacagctgcctagggtagatcttgagaagtagaaatgaaatccatgtagaatttcactgtacaccatagtgttggccactgtgtggctattggattatgtctgggcatgggacaattcacttctatcccggtctatcactttagcatttaaatgtgacccaagttgttggtgacagatgattggatggttgtccaaagctttggtttaacagtatggtggcgatgttcactctcaggtacatttaaaagtgattttgagaggcaggagagatagcttagcggttaagaacactggctgtgcttgcagaggtcctgagttctactcccagcacttgacatggcattagtttgtaactcttgttcctgggaatctgatgacctcttctggcttctgtgggcactgcatccacatggcacacatacatagtaaataaataaataaatatgcaaataaataaacaagtcaataccgcatgccacatacatgtaagtaaaacacacagacacacacacacacacacacacacacacacacacacacacacaccaaaccaatcaatcaatcaatcaatcaatcaatcaataatacatgtctctgctccccgattagcctgcaatgttggtgacttctcggcctgacattcatgactccctagttgtcgatttcttttgaacttcacttattaagtgtctctgattttggctgcaaccagtatttttctttttgcgggactgtgacaagcaggtggctttgattgcttgcatcactcttgattgttcactatctcctttctgttctctctctcatctttgctctatcaaaacgtctctgtcagtttctttgaagagactcctctgtttgtctatacccctcatggcctatgctgccttcgggggtctgtgtgtggtagttgtgatggcagcaccggcataggacgtgctggatagttcctgtgccgtgagttcttctgtaaagtctccattgcagccttcaagaggaggtattcttatttccccactttcactaacagaatcaccaatgagaggcgaaatactagagccccagctcatgctgcaaggactaacccttcaccatagttccctgccatgcactgcagtctacgggatagccatatcactttgcactggagtcatgtcttagattgcaggtagggtaggaggtgtggacatttatctatagtcaaacttgccacgagccttccaatcttccagcttcctccggagcaaactggaattgagcaagagttggttatgttagagcctaggatctataacaaaggcttatctttaaatagagtcactaataatgggtagatgcacatactcctaagaggcatgtgaagtcttgaagccgtttgaaggaagagccttctgcacctgacgatcaaagttacatctttctgagcactcactgccttatcatgttaaccctggaggctgactagcatggagatctactcccaggtcttttattgcactcctttaaaaagatttatgatatcatcttatgcattgtttttgagacagggcctactgtatctcaagctagcctcaaattcactagctaaggaggagcttgaatttctgatccccctgctttacctctcaagtgcaggaagtacctccgtgcctggtttatgtggagctgagattaaacccaggatttgttgtgtgcaaggcaagccctgctccaacttcatccccaggccaagtttatgtattttaagcacaaagttgaatagagttaaatttggcatgggtgcggactaatcgtcatccaacctccataactgtcttgtgctcttgtgtcttacgtgatgggctggggagatgctcagtgcttgctgtcttttaactcctgggatcctgtggtaggggagaacagacttctacatgttaatctttgacttccatcatgcgtcattggtacatgtgcacatacacacaaaataaataccagtaaatgtaataataaatataaataaaattgaaaattgaaagcactactaaggggctgagtgatgtctcagtaggtaaagtggttattttgcagggctgacatttgaatccccagaacccacataaagctatgctgtagtgcaggcatgcccctacttggagactggagatggagacaggagaattgctagaagcccataggccagctagttttgtatacacaacagtgaacagcaagagaaaccatgcctcaaacaaggtgaaaagcaaggatggacacctgagattgttctgtgacctgcacacatggaccctcttacatgtaagaggcctcacatcatggatacacacatacatgtgcatgcataagcacacacacaagagtactgtagactgtttcatgtcactgtttcttgagatgactagggaggggaacaaactatcgctggaaatacagaattgcaaatgttcttcctatacaccgtgtgctcagcaccatacacattaagcattctgaatgcactggcttcctgtccatatccctttaggatgaatgctgtcactctcatctcatctcatagactagaaagcaagatggagatttctggtgccttgatcttgatcacccagtccaaggctggctgagccactatgtaaacagtggtacagctttgggttcaaccacctcaacgcctcacattccagcgtctctgctggtttctgagggtgttgccactgaaaacttcaggtttcagatggctttttgttcttgaagagtttgggacaatggctctgcagaaatatttggtctaaccatccttccttctgttgcaaatacatccagtgggtacgtctgagggcccccccccccacctccaggcttttattaaccatggactgcttaggactccagacacaaacatcccagtgctcccaggctgggaagcaaatgtacttacctaaatttgaggagtaaacagttgaaaaaaaaatcctgctcatttaaaagtcctagttcctggaatatgcgcgcacacacacacatgagtcctttttcctggtttagaccataaatgaatggtatgaacaacatccttctttaaggtttaagggtctaggaggaagggagaaagggagagttcgggaggggaggagggaggggaagttccgattgggatgtaaagtaaataaataaatgtattaataaaaaataaacaaaagtaattaagagtatgtttaaatgtcaaatataacattattcatgatgaaggtgaagttcatcagagattcatttgtttcctcattcatataaaataacaagatcaaatagagtgagaccagaaaaaaaaaaagatctaagagtcatagggaaggtgagagcaaaagagcttgcttattatatatgattattgttttctcaggggaagaaataggtgctggctactgcacagttatatgtcagcagtgtgctccctttggaatggcactctagtttggcactttattttgagggaagctaatgcagactgagtcccattgtttgtttgtttgtttgtttgtttgtttgtttgttttgagacagggtttctctgtgtagccctggctgtcctggaactcactttgtagaccaggctggcctcgaacccagaaatattgcctctgcctcccaagtgctgggattaaaggtgtgcgccaccacgcccggcaagaatcccattcttagactgtgtacgaaagtacctttatgtctttccgcatctgttagtgtcccacacctcttccggacaaattcacttgtcaccactggacgcaagcttattccaatgtctaatctaaattattaaaattctttattttagaaaaaaacatattgagagcgtacagatggtgcagcaggcacggcagtaacaatgtctccctgctctgggccacttggattctgggtgttaggcccatggggcgtctctcatgcgccctctgcagtgctcacttcctgttgagattctccagcttcgggttgagctcttccagcttacaagcacgatccgtacagttcttattatctgaatgattaagaacaccacaaatatggataacacaagactgtttctgtttgcttgtcttgtttgtttttttaaaaacttaatgaatcaatcatctcataggtttacctctgttttatgctggaataaatataagccaagtagaccatggcaattagccctattacttaaaatctacagccaggttaggactagatgacaagcatctaggtttcaggtctggtagggttttagggtgcgatgtgtagtgtttgcatagcgaagaggtggaaatttatttatttatttattcattcatttatttatttatttatttattttgttttttgagacagggtttctctatatagccctggctgtcctggaactcactttgtagaccaggctggcctcaaactcagaaatccacctgcctctgcctcgaaagagtataaatgccgcttacagtggaaactggagggtcatgtgctgttcgttggccctgcacctttcaggagtctacactgtcgctgttctttgttgcctttgtaactgttaaatatttcttgttctacattagatctgctccgagatgatttggagtcctcagagttacatggcggagttgtcactgaaaaattctgtcctcctccctctccaggtttcatcgcttcagacatgacctccagacactccagtacccagctgtggctcatcacacattaccatgaaatgggatcgttgtatgactaccttcagctcactactctggatacggttagctgccttcggattgtactgtccatagccagcggccttgcccatttgcacatagagatatttgggacccaaggcaagcccgccattgcccatcgagatctgaagagcaaaaacatcctggtgaagaagaatggacagtgctgcatagc

SEQ ID NO: 11 - DNA, 17166 bp, synthetic construct
  misc_feature 1..17166: note = synthetic polynucleotide representing the 8432 Allele
  source 1..17166: mol_type = other DNA
aggggcgaagctcatagaactgaattgtttctcctcctcacctctcctttgcagatgagaagcctaaggtgaaccccaaactctacatgtgcgtgtgcgaaggtctctcctgtggtaatgaggaccactgcgaaggccagcagtgtttttcttctctgagcatcaacgatggcttccacgtctaccagaagggctgctttcaggtttatgagcaggggaagatgacgtgtaagaccccgccgtcacctggccaggctgtggagtgctgccaaggggactggtgtaacaggaacatcacggcccagctgcccactaaaggttcccatggctccttctattactagatcaaggggttttgtgagggttgccccaagtctgtgtgggttaaggatctgcgtagccagaagcattcttccgacttgctgagccagagctgctgggccatggaaggtcactctgggtgaggaagacaaaggcatggcctttgggattgaaaaggagagtgatgttttggggaaagtgagtgatgtgacatgcatccagaggaagggtgaagctcgagtaactttaaataatgccaattatttaaagttatgtacataactataacggtcctaaggtagcgagctagcggcatgtgtgtttcgagagaagtaggaacactagcttggtgtctgtgtgaagtgcctttcccttgttaagaagaagatgaaggaagccttgcccacccttccaaatcagatccctagccttctgaaacagaagtcagatgagggtgtgtatgagccctgaagttatgccatttcccctcatgtatttcttatggattctgaaccctttggaagtcggtcttcctctgaaactctttttctcatatggtgaactctcattgtcttatctcacattactgtcaatattaatataggctcaccccaagccaaatttattcttgtaatgtacagtacctcttaataggtttaattatatgattttgtccttgtggaagagggaaagcacaaataggataagagaggacacacacagccttcattgctaggactgtcacaaggcaggaaggaggcaagaggctggaaaccaaaagaccaataggtcacatggcagttttgaagttactatgaccccctttgggtagatagaacaggctacattgggatataaaaataggatcaaacacaagggtttgaaaaaggattggctatttgagtaattaactcacaggtcatttccttatgctcactgagttaatcctttgctaaaagaagggatggacaggaagaaacatctgtattcacacaaaaaccttgccagcagtgtgaagatcctcacgaacacattatgctgcacactacgcaacacctttacttatgtgttgacttggttgtgtttgtgtgtgcgaaagcgcaggcacacatggccctgcatttgcacatgtgtgttgagcatgtcagaggacaacttgtaggaattggttctcttattgtgtgggttccagggatcaaactcaggctgtcaggctttgcagcaactcctgagaccctctgagccctttcactctcccaacacccagtgttttcacagaagacatttcctggaaggaatgtcctatttgagaggccacaatatctgtcacttattaaatcacttttacgaataattatacagatggtatgcactatagagcaccggcctgattctcaggacttggggagttaaagaaacaatataaaggaacctttaagtagagagtggatggctgcagggaactctgagacaggtcaagcttagggaggagatcagcgctttctcactgggatattacagtatagaggctgttcatcagggctcgttgaaaagcatctttgcatctgaagtccacgttcataagcgttaacatcaaggattatgtgtctcttatatttaagaagctcttaagcattcatgcattaaaatacagttgattcttggtggaatcctttctaaaccccagttctatttaattgatcacttaaatgagtgtctgggccattgtctaaatgttaggaagtattttagttgtgaattgttttgatacgtgagatggtttggtcgggatcaggagccattacctaagatattggtaaataacaaagaaaactgtggtctccacgcctacttactcttttcttcccattctccagggaagtccttccccggaacacagaatttccacctggaagttggccttatcatcctctcggtggtgtttgcagtatgtcttttagcttgcatccttggagttgctctcaggaagtttaagagacgcaatcaagagcgcctgaaccccagagacgtggagtatggtaccattgaagggctcatcaccaccaatgtgggagacagcactctagcggtgagtttgtgaccccagagggaggcctacgaataacgagatggaggtggacactttcttgttttgaatgtgcaaagccaacaatgactatgcagacatgtatggtgactagtcactgggctaaaagccaaggacatccgtttcagacaggcacttaagggatggggctaggagattttagtagatccttggtggtgacttttctttggagaatgtgaggaaaagggacaaagaatagttggtggaccagagattcaaagaacttgacgtggtggtcttcagttactctatttgtaacaacaaacattttttttaaaggaagcatcttttcattattattaagaaatccttttccctttagattatgtgcatccgccataatttagagacttgacatctccagaagtagccattaagattttgatgctccagtactgcccccttgtggcgagtagtcggctggttgaaccagggtcctctagtacaactgggcatagcacatacagagtttaaaatgcttgagtagccagcttaacaagtaaatagggggaaaagcaaaccgtttataccttttatcatttcaacatacaataaaattgttgatatatttgataagttttcaaagtcagttacctggtgctaggaaccccccccccccccccccgagtttacttcctcagcatagaagatgctattatttatacatttggaaatttcttcctggcccgtgaggcggctccgagggtaaagatgctcactgctgagcctaatgacctaaatttaatccccaggactgatagggtaggaggagaaaaccaactcccacaagctgtcctctgacttccaaatatgtaccgtggcaaggctttccccctacccccaacaaataagcaaacaaatatgtgcaataaaaatttaaaattactcctgcattttaaagctatagcacgatactgtaagtgttcctttagttactcttctaaacttttagttaactcggggaagtaggtcactcaccataagcagcctactatagagtgcagaagaataaagtaacagccttctccatggatactacatggtttctctgcaaaagacaaagctctgtgggtgttaggtgcgaacaacacaaccaaatcagctctttccttgtcacaatctagctagtcgcaagtcaatcaagttatctttgtaatgtgattcagagctgtttaatgctttgctccctcctcatcccagatcgtcatgcccgcaggctggcttcactacctgacaagtccgtaagtgcctgggaattctgtagctaggcttcctctacaactgccttttgttgcttttctggaaaagatgtacgctaagcagtggtcatctttggctcacatgctagacctgggcagccataggtaccccctgtagtcaggtgtcagtacgttaataatgctggatccaggctgggaactgtcttgccctgattcctgtcccatcagggttctgtttgaatgaaagaggaacaagttagcctgtgtacatcgttttgcataccaatttgagttgtgatgtggtggaactgggagaaagggtgtaccttgtttccatgaaatgtttgtccagaagtctggtttaaggtgtaggggaaaggaggtggggtttatgagatttttgtagcagaaagacatgacgatgattcccgacaggcaaagcaagatgctaatgtagattagtcagaactgtgtttctccgagctgctagcatgtctaattaccaagggcagtaattggaatatctagtgtaaattactgtttgaatagcttttaggattttgaaatgggaatgaaagcctgcgccataacagtcttctcaacacagcccagccacgcagctgacagagtgtcaccatataatgacacagtaaggaataacgagatgtccttatttgcttatttaaacaatatgttcatttatgtgagctgacaaagagtaaccttgctgaagctcgcccacggctgggccttcagaacttgaggtaggtaggtctttattagtctttgcttgattgcctgatgccccatctcccaaatctgcacacttaattagaaccaaaactatgcctcatggaaaagcaacagctcaatgtgcagagggttttttttttttttttccctcaaaaataattgaacgaatagagtcaccaagcagggcaagagggaattgttggggagacacgaagcctatttcatcacattttttttcttcttgattatccttttgtgacttttaggggttctgaatttcttatgttgagtgtttagcttttggtaatcttactgatccacagtatctcacggtcttgctccacagaaaggagagaagatgaaattcacagaaatctatgtgttgctttaatgggccagtttatgtgttgggaaaggaagaactttgggctgggttaatctttccaccatcaccatggctataaggctgtctgtgccttagagatagtccaggccgggtcctgccgctgggcctttgctgagagtgtgaaagaaaacttcatgaatgaactaactggcagccttttgccagtatgctcatttgggaaatcaatgtgcgactctggtgttatgttttaagtgaaatatatatatattttttttcctgtttcctttctaatttgcagttcatatggaaaaatgataagtaggtgttaatttagtgacctgcttgttcatatattacctgaaaaaaatgagaatcttcatccacagaaacacccacgcacacacctataagtaccaatttaatgacccacatattattagaatcttttcattacagatattacacttggctcttcatagtcctaatttgcttgcttccaattagacacctttagtttgtaaggcaaccgaagtagatcacttgatagacatttagtggacatagcagaatagataatagtagacaatgcccagctcttggtgctcaagtatcccccacccccattcttcctactgactcctcacgcacagccccgtagcctttctaatgtgcgcgttagcttagctctgtttccttccttgcatatttgattagaaaactctatagagcttaagaaagaatgacttatccatcaggaaaacctgctgtaggtttcgcttctaagtttcccaagacatataactagtcctttcaaaggagattttcaggcatttgaatgggtcacatgtccagagttgaccggtataacttcgtataatgtatgctatacgaagttatggtacctggaaatcctgtgggttcccacagcatgtattgcaggacgctgaagcgggcactgttgctcagtgatttctttttctccttgttttacatcacaggaactactagatcactcgtgtacatcaggaagtggctccggtcttcctttcctggtacagagaacggtggctcaccagataaccctgttggagtgtgtcggtaattctatgtttcccttccttttgggcagcaggcaatgttggttgtgattctcgaagtcagccagggcttggaaaagtctgcttcttgttgcttattattgcttcctcattaaatgcaggaggtgataacttcgtataggataccttatacgaagttatggatccccaagcaaccatgtggttattccacttcagggggtgtgagacagccatgggggtctctgttctgtgtgactcacggttgagagcttgctgaagttgtggtaagagagtcatccctgactagaacagacaatgcctagcgtgaatccacgaggctgcgttgaacaggctcgagaagttcctattccgaagttcctattctctagaaagtataggaacttcgctagctcaagttactgtgtatcaattagacacaagagcttatgaaaatatacatttaagaactcgagtgaaccttactatgtttttttttttaattaagtgcttagtacgaagacctatatacattttgatagcctttcatttttctccctatgacatagtgtaggtcagcatattaattgtgaaaaaaattgtactcctttatccgttaaaatttctaaaaaaaagaaaaaaatctgctgttgctgcatgtgggtatatgacgtatgtgtgtgggtgtgtgcacgccacagcacacgtggactttggaggacagtgctgtagagttggttctcacaccgtacctttatatgggctctgggggttgaactcatatcatcaggctttgcggcaagcgcttcacccattgaaccacctcagaggcccaccttccttcattgttcagaatgcacacacctcgtctggtgttgggaattatcagaagtactttaaaaagcggtgcaacggtcagttcggctagcaggttagcagagatgcgtgaaaaggatttggcacacattctttggtcttctgaattggcagaatgaattccaggaattgtgaatttttgggtcaggcatgatttaagaataataaaatgatgatggtgacggcggtggcggcttcatgaattccatgaggctggtacagaatgctcttctagcttttgagacagggtctcttctggcccaggcagacccaaaactcggtatctagccgagaatcgtcttttgatgctctctcctctctctcttgagtactgggggttattataagtatacatccctatggccaaatagagttttaaagtcctgattttcagggctgaggggatggctcagataataagtgcttgttgcacaagcatgaagactggattttgatccccagtgcctatgtagaaaagcttgcacagtggcatgtatgtatgtgcctatagtctcagtgctggggatgcagaaagggcaagatctgtggagtttgctggccaagcagtctaggtttatctgtgagccttaggtttagtctgaaacctgtctcaattaagtaagtgactgagccaaacacccagtattagcgtcacgtatatatacgtagctgccctccccttcccattccccccacacatgaaggacacactttattttacacttgatcttagccaaaaggccgagaagcgatcacactttattttaaatacaatcctcagtttcactatttgtatgtggagtgtagatatttgcaggtagcatgacgggttttgtcagcttctcattgagatggtgtgtaggaatgccttgtattgtctcaccccttctggtgagcaggctttttgggtggctatagcagctcctgccttctgacagtctgtttggcggacgctgtcgcaggctatttgatcacattgtgaatcagaaaaaccctgtggtggtgtggctcagccctagcacacacctttaatccaaagagctttcttcgaatggaattataaagtcagccacaggtcaagaggcagggcaagcaaccagttgacagggagtgaacataggaaaacccaggaaagagaaaggcgggtctttgagttgaggggtatctaactatatcatggaggagaaagagccttttttcatctgggacatggtgaactaggaaggtcagccgggtgctttctctgcctttcggagctagcaggctttcaccacagcatctggctttggagtcttcattcgggataccaaatgtttgggattttgttttaaaaacaacaggaagcagctgaccattatgaactggtccctgcgctttctgccatatgaaggaggaacagtgtcaaatctagtagtaagatctgcagtgggcagcacggcttgcctgtgacacacaggggccagcaaaggagctgtggtaccttttccccaggtctgcacctactgttcctatttcccattctctgcagctccaccacaactgactaagactagaaagagatggagaaacaaaacaaacaaaacaacaacaacaaaacagaggcactcagcacccacaaccagaatggttgtcttgacgtctaactggcatcctgtttgtgtatgaacaaggatacctgggtagtgatggcaggcagcagaggggccatgcacctgctgctcctgtcttcttgccccaacaacccatcactcacagatagtcccacatacctcagagactggctccagagtcttgctcctctgaactgtctacagtgtcgcactcgatagatgctcttcaacatttttcaccactgtaaatttaactattgcattttgggggttgacattagagcctggtgggtggagagcctgttcagaagttttttgtttttgtgtccaaggtgtagcttggcgggaaaagatggatagatgatcactttagttcaagaacgtggggatctgaccatgtcaacccttccagccctatgcatcctctttgtgggactttctggccagggacttttggggaactgtaccactcttttagtggctttgacataagattacattagcaaaaagcatttgacagccattaaaatagatttctgttctgatttaatccacctaccatgtcgtatattttcttttcttaacttaaaaaaaaaaaaaagatttacaacaacaaacaactttgtatccacaattctttgggatgtttttcttgccttctcagtgaaacagtgactccccagagaaaatcaaaaagttgcaagatggaacaaaggcaggaaaagcttagcatttatttcctgcttttgtagcttctggcttggtggcatcttcaggcagttagtcctcatcttgaacagaaaggttgagccactcctacgacttgattaaaatgacttgtaaaccaagtagactttgtgtgattattgacagtagctcacaaggaactctggcctctcttagcatccctttatattagtcattgctattggatagggttatcagttcttgtcatggaagctcagtttgctggatatccgtgctgccacctctggagctggatggtacccagtgcctcacacctgtgaggcaggcactctatcgacaagtcgcatcccctgcccttggagcttttgtgacagtttcaggtatgtcactctggcacacctgctttctttctctgtgtagggtcttattggcttagttttccagcagaagattgtacttgtgtgcaggcaggtggcaccacagctgcctattacagccccgtgatggtgcagtcaccacatgcctgggcagaaagcctcattgcactgtagtttcggttcccgctgcacgcctgtcatccaagccgactacacctcatgttgacccccactggttttgagataatgacatcctcttccctatctctttgatcatctctacctataattaaagagatgactggaagggggacatgcccttgataagcatgtggctactacatagaggtggggggaggggcatgaatcagacaggtcagatcccagctgaagttaccctattttgtaaactatgctcttcaagttactaaaatgaaatcctgaattatattaagtgtataggggttttgcctgcatgtatgtctgggtatcccaagtatgcctcgtgtttgaggaggtaagaagagggcattgcattccctggtactgatgtaataatagaagggtgtgtgctgcgtgggttcttggaatcaaatctgggtcgtccggaagagcctccaagactcttaacccctgagccatttcttcagccctcgggtgacttaagaaatgttttatctaccagttttgtgtgtgtatgtttatggcgcacatatgtgactgtgctttgtggagtcagttctctattttcacttttatatagattctagagtccgaacccaggatcacacttggcaagctcatttgcttgctggtactgtctttctaactgccaaaggctctttattttagtgtagtctgagtgtgacatcactggggcacatctaaaccaacaccctgaccagtgtcagaagttagagctcttctggaattatgtctatgactttgaaactctgcttttgaaatatatctttctccaagaaaccccttggaggtggtggttcacaaccttctaccagcactggggactgtcattaatctccaggatgtcaaactgcccctctgtggggcaagaggagcttaaatatattcaggcatttgggctgagaaatgcttctcagggtccagtggaaagggggggaaccagagaaaccatctagttatttagatatatacccctaccccttgggcttttgatgctacattgaaatttagtgatcagtagtatgtgctaaattgtactacctttcatgtgttttgaaaaactagatgccaaaagtttctagccttttgacattgtgactttccactgtcatctacaaacaggttgagctgcctgcatagttagtctggggcacaagtcagacaccctcaggaaagcaacttaaatataaatgtgaagttagttatcaggtactgtactgactggaaggcagattcctgccatgattcaactggtagggaaagataggttttaccttatttttgtacatctttataggtgaagtatttgcagttctagactgagtttgtctagcaaataatgaagagtttggggcatcaggctttgtttactactaggagtttcttggtcaggctgtgtgtcctgtctttacccttctatacaacaaagaataaattcagtgtcacacaaagaaacacagaaccaatgcaaagttattggcatggcagtttcttttcaaaaaggatctaccagaacccaaatccagctagtgaactcatgtgccaaatggcctccatattttatttttgatacaggtggtgactttgttctatgccatgggtgggagcttggcttcacagtctgacataattggttagtcagaagagaagagcaggaggttcaaatataaggacactgtcgggttcactgtgtttgagaggaaaaggaaagccaacccatttcttacatatatcatctgttttgctgtccacagggaagggccggtatggagaagtatggaggggcagctggcaaggcgaaaatgtcgctgtgaagatcttctcctcccgagacgagaagtcatggttcagggagacggaattgtacaacactgtgatgttgaggcatgaaaatatcttaggtgagtaccaggtgagctttcaccagctggtctccatagagataagggccggccctttctctctcccatttgccaaatctgaggtgtgtggagtctcttctggaaaacaacatagtaaccctcagcatgtatagactacttaattttttgacaggtatttttgtttgtttgtttaaatctgcacataggttatgaattgcctgcatatgaaaaggaaatccacagcaggacctttctcctcctgtatctgtggatgtattgaagcaaagccctgctctgctccgttgttcacttcaaggctccttgggtggttctgcttgccccacccttcctgaatatttattactggaatcaaactaagaaaggtctttggattctttcctagagattcacagctcccagggacgcgtaattagatgcacatttcctcaaactaggcatcatttttaaagaccagatttacagagggattcagcatacattttaatttgaaagctcctcgccttacttctgggactcacaaagtatagatgaattttcatcatcttatcactaaagcttagcaatgcttaagaaatattcgaggtgcctactacaatcctgctatggcaatgagagggtttccatagtttcaaaaggctttgagtgtatttccattgatacacacatatggtgacagctgcctagggtagatcttgagaagtagaaatgaaatccatgtagaatttcactgtacaccatagtgttggccactgtgtggctattggattatgtctgggcatgggacaattcacttctatcccggtctatcactttagcatttaaatgtgacccaagttgttggtgacagatgattggatggttgtccaaagctttggtttaacagtatggtggcgatgttcactctcaggtacatttaaaagtgattttgagaggcaggagagatagcttagcggttaagaacactggctgtgcttgcagaggtcctgagttctactcccagcacttgacatggcattagtttgtaactcttgttcctgggaatctgatgacctcttctggcttctgtgggcactgcatccacatggcacacatacatagtaaataaataaataaatatgcaaataaataaacaagtcaataccgcatgccacatacatgtaagtaaaacacacagacacacacacacacacacacacacacacacacacacacacacaccaaaccaatcaatcaatcaatcaatcaatcaatcaataatacatgtctctgctccccgattagcctgcaatgttggtgacttctcggcctgacattcatgactccctagttgtcgatttcttttgaacttcacttattaagtgtctctgattttggctgcaaccagtatttttctttttgcgggactgtgacaagcaggtggctttgattgcttgcatcactcttgattgttcactatctcctttctgttctctctctcatctttgctctatcaaaacgtctctgtcagtttctttgaagagactcctctgtttgtctatacccctcatggcctatgctgccttcgggggtctgtgtgtggtagttgtgatggcagcaccggcataggacgtgctggatagttcctgtgccgtgagttcttctgtaaagtctccattgcagccttcaagaggaggtattcttatttccccactttcactaacagaatcaccaatgagaggcgaaatactagagccccagctcatgctgcaaggactaacccttcaccatagttccctgccatgcactgcagtctacgggatagccatatcactttgcactggagtcatgtcttagattgcaggtagggtaggaggtgtggacatttatctatagtcaaacttgccacgagccttccaatcttccagcttcctccggagcaaactggaattgagcaagagttggttatgttagagcctaggatctataacaaaggcttatctttaaatagagtcactaataatgggtagatgcacatactcctaagaggcatgtgaagtcttgaagccgtttgaaggaagagccttctgcacctgacgatcaaagttacatctttctgagcactcactgccttatcatgttaaccctggaggctgactagcatggagatctactcccaggtcttttattgcactcctttaaaaagatttatgatatcatcttatgcattgtttttgagacagggcctactgtatctcaagctagcctcaaattcactagctaaggaggagcttgaatttctgatccccctgctttacctctcaagtgcaggaagtacctccgtgcctggtttatgtggagctgagattaaacccaggatttgttgtgtgcaaggcaagccctgctccaacttcatccccaggccaagtttatgtattttaagcacaaagttgaatagagttaaatttggcatgggtgcggactaatcgtcatccaacctccataactgtcttgtgctcttgtgtcttacgtgatgggctggggagatgctcagtgcttgctgtcttttaactcctgggatcctgtggtaggggagaacagacttctacatgttaatctttgacttccatcatgcgtcattggtacatgtgcacatacacacaaaataaataccagtaaatgtaataataaatataaataaaattgaaaattgaaagcactactaaggggctgagtgatgtctcagtaggtaaagtggttattttgcagggctgacatttgaatccccagaacccacataaagctatgctgtagtgcaggcatgcccctacttggagactggagatggagacaggagaattgctagaagcccataggccagctagttttgtatacacaacagtgaacagcaagagaaaccatgcctcaaacaaggtgaaaagcaaggatggacacctgagattgttctgtgacctgcacacatggaccctcttacatgtaagaggcctcacatcatggatacacacatacatgtgcatgcataagcacacacacaagagtactgtagactgtttcatgtcactgtttcttgagatgactagggaggggaacaaactatcgctggaaatacagaattgcaaatgttcttcctatacaccgtgtgctcagcaccatacacattaagcattctgaatgcactggcttcctgtccatatccctttaggatgaatgctgtcactctcatctcatctcatagactagaaagcaagatggagatttctggtgccttgatcttgatcacccagtccaaggctggctgagccactatgtaaacagtggtacagctttgggttcaaccacctcaacgcctcacattccagcgtctctgctggtttctgagggtgttgccactgaaaacttcaggtttcagatggctttttgttcttgaagagtttgggacaatggctctgcagaaatatttggtctaaccatccttccttctgttgcaaatacatccagtgggtacgtctgagggcccccccccccacctccaggcttttattaaccatggactgcttaggactccagacacaaacatcccagtgctcccaggctgggaagcaaatgtacttacctaaatttgaggagtaaacagttgaaaaaaaaatcctgctcatttaaaagtcctagttcctggaatatgcgcgcacacacacacatgagtcctttttcctggtttagaccataaatgaatggtatgaacaacatccttctttaaggtttaagggtctaggaggaagggagaaagggagagttcgggaggggaggagggaggggaagttccgattgggatgtaaagtaaataaataaatgtattaataaaaaataaacaaaagtaattaagagtatgtttaaatgtcaaatataacattattcatgatgaaggtgaagttcatcagagattcatttgtttcctcattcatataaaataacaagatcaaatagagtgagaccagaaaaaaaaaaagatctaagagtcatagggaaggtgagagcaaaagagcttgcttattatatatgattattgttttctcaggggaagaaataggtgctggctactgcacagttatatgtcagcagtgtgctccctttggaatggcactctagtttggcactttattttgagggaagctaatgcagactgagtcccattgtttgtttgtttgtttgtttgtttgtttgtttgttttgagacagggtttctctgtgtagccctggctgtcctggaactcactttgtagaccaggctggcctcgaacccagaaatattgcctctgcctcccaagtgctgggattaaaggtgtgcgccaccacgcccggcaagaatcccattcttagactgtgtacgaaagtacctttatgtctttccgcatctgttagtgtcccacacctcttccggacaaattcacttgtcaccactggacgcaagcttattccaatgtctaatctaaattattaaaattctttattttagaaaaaaacatattgagagcgtacagatggtgcagcaggcacggcagtaacaatgtctccctgctctgggccacttggattctgggtgttaggcccatggggcgtctctcatgcgccctctgcagtgctcacttcctgttgagattctccagcttcgggttgagctcttccagcttacaagcacgatccgtacagttcttattatctgaatgattaagaacaccacaaatatggataacacaagactgtttctgtttgcttgtcttgtttgtttttttaaaaacttaatgaatcaatcatctcataggtttacctctgttttatgctggaataaatataagccaagtagaccatggcaattagccctattacttaaaatctacagccaggttaggactagatgacaagcatctaggtttcaggtctggtagggttttagggtgcgatgtgtagtgtttgcatagcgaagaggtggaaatttatttatttatttattcattcatttatttatttatttatttattttgttttttgagacagggtttctctatatagccctggctgtcctggaactcactttgtagaccaggctggcctcaaactcagaaatccacctgcctctgcctcgaaagagtataaatgccgcttacagtggaaactggagggtcatgtgctgttcgttggccctgcacctttcaggagtctacactgtcgctgttctttgttgcctttgtaactgttaaatatttcttgttctacattagatctgctccgagatgatttggagtcctcagagttacatggcggagttgtcactgaaaaattctgtcctcctccctctccaggtttcatcgcttcagacatgacctccagacactccagtacccagctgtggctcatcacacattaccatgaaatgggatcgttgtatgactaccttcagctcactactctggatacggttagctgccttcggattgtactgtccatagccagcggccttgcccatttgcacatagagatatttgggacccaaggcaagcccgccattgcccatcgagatctgaagagcaaaaacatcctggtgaagaagaatggacagtgctgcatagc

SEQ ID NO: 12 - DNA, 20 bp, synthetic construct
  misc_feature 1..20: note = synthetic oligonucleotide
  source 1..20: mol_type = other DNA
gcatccagaggaagggtgaa

SEQ ID NO: 13 - DNA, 15 bp, synthetic construct
  misc_feature 1..15: note = synthetic oligonucleotide
  source 1..15: mol_type = other DNA
actgcaggtcatcac

SEQ ID NO: 14 - DNA, 20 bp, synthetic construct
  misc_feature 1..20: note = synthetic oligonucleotide
  source 1..20: mol_type = other DNA
acatgccccatacacccatt

SEQ ID NO: 15 - DNA, 24 bp, synthetic construct
  misc_feature 1..24: note = synthetic oligonucleotide
  source 1..24: mol_type = other DNA
ggctgactgatctgaaggaaatgg

SEQ ID NO: 16 - DNA, 26 bp, synthetic construct
  misc_feature 1..26: note = synthetic oligonucleotide
  source 1..26: mol_type = other DNA
tctggatagtaaggtcagttgctgcg

SEQ ID NO: 17 - DNA, 23 bp, synthetic construct
  misc_feature 1..23: note = synthetic oligonucleotide
  source 1..23: mol_type = other DNA
agaggaaggagacgctaagaatc

SEQ ID NO: 18 - DNA, 24 bp, synthetic construct
  misc_feature 1..24: note = synthetic oligonucleotide
  source 1..24: mol_type = other DNA
tgaaggaaatgggcttctggatag

SEQ ID NO: 19 - DNA, 23 bp, synthetic construct
  misc_feature 1..23: note = synthetic oligonucleotide
  source 1..23: mol_type = other DNA
aaggtcagttgctgcgtcttccc

SEQ ID NO: 20 - DNA, 25 bp, synthetic construct
  misc_feature 1..25: note = synthetic oligonucleotide
  source 1..25: mol_type = other DNA
catactcactcttcctgttagagga

SEQ ID NO: 21 - DNA, 21 bp, synthetic construct
  misc_feature 1..21: note = synthetic oligonucleotide
  source 1..21: mol_type = other DNA
ccttgcccatttgcacataga

SEQ ID NO: 22 - DNA, 15 bp, synthetic construct
  misc_feature 1..15: note = synthetic oligonucleotide
  source 1..15: mol_type = other DNA
aagggaagtccgcca

SEQ ID NO: 23 - DNA, 24 bp, synthetic construct
  misc_feature 1..24: note = synthetic oligonucleotide
  source 1..24: mol_type = other DNA
tgtttttgctcttcagatctcgat

SEQ ID NO: 24 - DNA, 23 bp, synthetic construct
  misc_feature 1..23: note = synthetic oligonucleotide
  source 1..23: mol_type = other DNA
tttgcagatgagaagcctaaggt

SEQ ID NO: 25 - DNA, 18 bp, synthetic construct
  misc_feature 1..18: note = synthetic oligonucleotide
  source 1..18: mol_type = other DNA
caaactctacatgtgcgt

SEQ ID NO: 26 - DNA, 20 bp, synthetic construct
  misc_feature 1..20: note = synthetic oligonucleotide
  source 1..20: mol_type = other DNA
cgcagtggtcctcattacca

SEQ ID NO: 27 - DNA, 21 bp, synthetic construct
  misc_feature 1..21: note = synthetic oligonucleotide
  source 1..21: mol_type = other DNA
ccttgcccatttgcacataga

SEQ ID NO: 28 - DNA, 15 bp, synthetic construct
  misc_feature 1..15: note = synthetic oligonucleotide
  source 1..15: mol_type = other DNA
aaggcaagcccgcca

SEQ ID NO: 29 - DNA, 24 bp, synthetic construct
  misc_feature 1..24: note = synthetic oligonucleotide
  source 1..24: mol_type = other DNA
tgtttttgctcttcagatctcgat

SEQ ID NO: 30 - DNA, 115 bp, synthetic construct
  misc_feature 1..115: note = synthetic polynucleotide
  source 1..115: mol_type = other DNA
cctctcctttgcagatgagaagcctaaggtgaaccccaaactctacatgtgcgtgtgcgaaggtctctcctgtggtaatgaggaccactgcgaaggccagcagtgtttttcttct

SEQ ID NO: 31 - DNA, 68 bp, synthetic construct
  misc_feature 1..68: note = synthetic polynucleotide
  source 1..68: mol_type = other DNA
cagaggaagggtgaagctcgagtaactttaaataatgccaattatttaaagttagccagtagcagcac

SEQ ID NO: 32 - DNA, 106 bp, synthetic construct
  misc_feature 1..106: note = synthetic polynucleotide
  source 1..106: mol_type = other DNA
cgacctgcagccgctaggtaactttaaataatgccaattatttaaagttatgtacataactataacggtcctaaggtagcgagctagcggcatgtgtgtttcgaga

SEQ ID NO: 33 - DNA, 1056 bp, synthetic construct
  misc_feature 1..1056: note = synthetic polynucleotide
  source 1..1056: mol_type = other DNA
ggtcacatgtccagagttgaccggtataacttcgtataatgtatgctatacgaagttatgctgagcggattgctgcccttcatgtgagttacaatgtcatgcatgctaatactccaaagtgggagctatattgctcaatcgtttcttttcccccttgtcttaaaccacaggaattattggatcattcgtgtacatcaggaagtggctctggtcttccttttctggtacaaagaacagtggctcgccagattacactgttggagtgtgtcggtaattcttttttttcctttctttgtgggtaatatgcaatgttagttttgtttttgaagtaaaagatggaacttggaaaatctgctttttgttacctgttgtttccttattaaacttatacgcgtataacttcgtataaggtatcctatacgaagttatcacctcctgcatttaatgaggaagcaataataagcaacaagaagcagacttttccaagccctggctgacttcgagaatcacaaccaacattgcctgctgcccaaaaggaagggaaacatagaattaccgacacactccaacagggttatctggtgagccaccgttctctgtaccaggaaaggaagaccggagccacttcctgatgtacacgagtgatctagtagttcctgtgatgtaaaacaaggagaaaaagaaatcactgagcaacagtgcccgcttcagcgtcctgcaatacatgctgtgggaacccacaggatttccaggtaccataacttcgtatagcatacattatacgaagttatccggacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggccgcgactgcgtacgataacttcgtataggataccttatacgaagttatggatccccaagcaaccatgtggttattccactt

SEQ ID NO: 34 - DNA, 93 bp, synthetic construct
  misc_feature 1..93: note = synthetic polynucleotide
  source 1..93: mol_type = other DNA
gaggctgcgttgaacaggctcgagaagttcctattccgaagttcctattctctagaaagtataggaacttctatgcatggcctccgcgccggg

SEQ ID NO: 35 - DNA, 86 bp, synthetic construct
  misc_feature 1..86: note = synthetic polynucleotide
  source 1..86: mol_type = other DNA
gaatgaaccggcgatatcgaagttcctattccgaagttcctattctctagaaagtataggaacttcgctagctcaagttactgtgt

SEQ ID NO: 36 - DNA, 334 bp, synthetic construct
  misc_feature 1..334: note = synthetic polynucleotide
  source 1..334: mol_type = other DNA
aaaattctgtcctcctccctctccaggtttcatcgcttcagacatgacctccagacactccagtacccagctgtggctcatcacacattaccatgaaatgggatcgttgtatgactaccttcagctcactactctggatacggttagctgccttcggattgtactgtccatagccagcggccttgcccatttgcacatagagatatttgggacccaaggcaagcccgccattgcccatcgagatctgaagagcaaaaacatcctggtgaagaagaatggacagtgctgcatagcagatttgggtaaactttaaaaagaaaatcttaattattgc

SEQ ID NO: 37 - DNA, 110 bp, synthetic construct
  misc_feature 1..110: note = synthetic polynucleotide
  source 1..110: mol_type = other DNA
cagaggaagggtgaagctcgagtaactttaaataatgccaattatttaaagttatgtacataactataacggtcctaaggtagcgagctagcggcatgtgtgtttcgaga

SEQ ID NO: 38 - DNA, 91 bp, synthetic construct
  misc_feature 1..91: note = synthetic polynucleotide
  source 1..91: mol_type = other DNA
gaggctgcgttgaacaggctcgagaagttcctattccgaagttcctattctctagaaagtataggaacttcgctagctcaagttactgtgt

SEQ ID NO: 39 - DNA, 671 bp, synthetic construct
  misc_feature 1..671: note = synthetic polynucleotide
  source 1..671: mol_type = other DNA
ggtcacatgtccagagttgaccggtataacttcgtataatgtatgctatacgaagttatggtacctggaaatcctgtgggttcccacagcatgtattgcaggacgctgaagcgggcactgttgctcagtgatttctttttctccttgttttacatcacaggaactactagatcactcgtgtacatcaggaagtggctccggtcttcctttcctggtacagagaacggtggctcaccagataaccctgttggagtgtgtcggtaattctatgtttcccttccttttgggcagcaggcaatgttggttgtgattctcgaagtcagccagggcttggaaaagtctgcttcttgttgcttattattgcttcctcattaaatgcaggaggtgataacttcgtataggataccttatacgaagttatggatccccaagcaaccatgtggttattccacttcagggggtgtgagacagccatgggggtctctgttctgtgtgactcacggttgagagcttgctgaagttgtggtaagagagtcatccctgactagaacagacaatgcctagcgtgaatccacgaggctgcgttgaacaggctcgagaagttcctattccgaagttcctattctctagaaagtataggaacttcgctagctcaagttactgtgtatcaa

SEQ ID NO: 40 - protein, 26 aa, synthetic construct
  REGION 1..26: note = synthetic peptide
EKPKVNQKLYMCVCEGLSCGNEDHCE

(19) United States
(12) Patent Application Publication
(10) Pub. No.: US 2025/0295096 A1
(43) Pub. Date: Sep. 25, 2025

(54) A RODENT MODEL OF FIBRODYSPLASIA OSSIFICANS PROGRESSIVA

(71) Applicant: REGENERON PHARMACEUTICALS, INC., Tarrytown, NY (US)

(72) Inventors: Sarah HATSELL, Tarrytown, NY (US); Aristides ECONOMIDES, Tarrytown, NY (US); John LEES-SHEPARD, Tarrytown, NY (US)

(21) Appl. No.: 18/860,104

(22) PCT Filed: Apr. 25, 2023
(86) PCT No.: PCT/US2023/066185

§ 371 Date: Oct. 25, 2024

Related U.S. Application Data
(60) Provisional application No. 63/334,773, filed on Apr. 26, 2022.
(60) Provisional application No. 63/375,494, filed on Sep. 13, 2022.

(51) Int. Cl. A01K 67/0278; C07K 14/705; C12N 15/85

(52) U.S. Cl. CPC A01K 67/0278; C07K 14/705; C12N 15/8509; A01K 2207/15; A01K 2217/072; A01K 2217/206; A01K 2227/105; A01K 2267/0306; A01K 2267/035; C12N 2015/8536

(57) ABSTRACT

This disclosure relates to a genetically modified rodent whose genome comprises a modified Acvr1 gene which encodes a modified Acvr1 polypeptide that is expressed in the rodent, causing the rodent to display a phenotypical feature of fibrodysplasia ossificans progressiva (FOP) such as ectopic bone formation without neonatal lethality This disclosure also relates to nucleic acid vectors and methods for making the genetically modified rodent, as well as methods of using the genetically modified rodent as an animal model of human diseases.

### CROSS REFERENCE TO RELATED APPLICATIONS

This application claims the benefit of priority to U.S. Provisional Application No. 63/334,773, filed Apr. 26, 2022, and U.S. Provisional Application No. 63/375,494, filed Sep. 13, 2022, the entire contents of both of which are incorporated herein by reference.

### FIELD OF THE DISCLOSURE

This disclosure relates to genetically modified rodent animals and rodent models of human diseases. More specifically, this disclosure relates to a genetically modified rodent whose genome comprises a modified Acvr1 gene which encodes a modified Acvr1 polypeptide that is expressed in the rodent, causing the rodent to display a phenotypical feature of fibrodysplasia ossificans progressiva (FOP) such as ectopic bone formation without neonatal lethality. This disclosure also relates to nucleic acid vectors and methods for making the genetically modified rodent, as well as methods of using the genetically modified rodent as an animal model of human diseases.

### INCORPORATION BY REFERENCE OF SEQUENCE LISTING

The sequence listing in the XML file, named as 41344WO_11136WO01_SequenceListing.xml of 119 KB, created on Apr. 25, 2023, and submitted to the United States Patent and Trademark Office via Patent Center, is incorporated herein by reference.

### BACKGROUND ART

Various publications, including patents, patent applications, published patent applications, accession numbers, technical articles and scholarly articles are cited throughout the specification. Each of these cited publications is incorporated by reference, in its entirety and for all purposes, in this document.

Acvr1 is a type I receptor for bone morphogenic proteins (BMPs). Certain mutations in the human ACVR1 gene, including mutations that give rise to the amino acid modification R206H or R258G, are strongly associated with the disease fibrodysplasia ossificans progressiva (FOP) in humans (see, e.g., US Pat. Appl. Publ. No. 2009/0253132; Pignolo, R. J. (2011) Orphanet Journal of Rare Diseases, 6:80, 1-6; and Kaplan et al., Am J Med Genet A. 2015; 167 (10): 2265-2271). Chimeric mice that bear an R206H mutation in Acvr1 develop an FOP-like phenotype (see, e.g., Chakkalakal et al. (2012) J. Bone and Mineral Res. 27:1746-1756). Certain mutations in the Acvr1 gene, e.g., those resulting in an R206H Acvr1 protein variant, are perinatal lethal in mice and present challenges for passing a knock-in gene comprising the mutation through the germline of a rodent.

### SUMMARY OF THE DISCLOSURE

Disclosed herein are genetically modified rodent animals suitable for use as a rodent model of FOP. The genetically modified rodent animals display features characteristic of human FOP including congenital toe malformations and injury-induced and idiopathic heterotopic ossification (HO) in post-natal life, without neonatal lethality.

In some embodiments, disclosed herein is a genetically modified rodent which comprises a modified rodent Acvr1 gene at an endogenous rodent Acvr1 locus encoding a modified rodent Acvr1 polypeptide, wherein the modified rodent Acvr1 polypeptide comprises the ectodomain of a human ACVR1 protein, and the transmembrane and cytoplasmic domains of an endogenous rodent Acvr1 protein except for a S330P substitution and an FOP mutation selected from a R206H substitution or a R258G substitution; and wherein expression of the modified rodent Acvr1 gene is under control of the rodent Acvr1 promoter at the endogenous rodent Acvr1 locus.

In some embodiments, exon 2 of a modified rodent Acvr1 gene differs from exon 2 of an endogenous rodent Acvr1 gene by comprising (i) a substitution of the codon for Q at position 30 with a codon for P, or (ii) a replacement of a sequence of exon 2 of the endogenous rodent Acvr1 gene encoding endogenous rodent Acvr1 ectodomain amino acids including Q30, with either a 5′ sequence of a human ACVR1 exon 2 encoding human ACVR1 ectodomain amino acids comprising P at position 30, or a sequence modified from the 5′ sequence of the human ACVR1 exon 2 to include one or more silent mutations. In some embodiments, the human ACVR1 ectodomain amino acids encoded by the 5′ sequence of a human ACVR1 exon 2 comprise amino acids from position 24 to position 49.

In some embodiments, exon 6 of a modified rodent Acvr1 gene differs from exon 6 of the endogenous rodent Acvr1 gene by comprising a substitution of the codon for Ser at position 330 with a codon for Pro, optionally by further comprising a synonymous nucleotide substitution.

In some embodiments, exon 4 of a modified rodent Acvr1 gene differs from an endogenous rodent Acvr1 exon 4 by comprising a substitution of the codon for R at position 206 with a codon for H, optionally by further comprising a replacement of a sequence of the endogenous rodent Acvr1 exon 4 with a corresponding sequence of human ACVR1 exon 4 wherein the replacement does not change the amino acids encoded by the endogenous rodent Acvr1 exon 4.

In some embodiments, a modified rodent Acvr1 gene comprises: an endogenous rodent Acvr1 exon 1, a modified rodent Acvr1 exon 2 which differs from an endogenous rodent Acvr1 exon 2 by comprising a substitution of the codon for Q30 with a codon for P; an endogenous rodent Acvr1 exon 3; a modified rodent Acvr1 exon 4 which differs from an endogenous rodent Acvr1 exon 4 by comprising a substitution of the codon for R206 with a codon for H; an endogenous rodent Acvr1 exon 5; a modified rodent Acvr1 exon 6 that differs from exon 6 of the endogenous rodent Acvr1 gene by comprising a substitution of the codon for S330 with a codon for P; and endogenous rodent Acvr1 exons 7-9. In some embodiments, the modified rodent Acvr1 exon 2 differs from the endogenous rodent Acvr1 exon 2 by comprising a replacement of a 5′ sequence of the endogenous rodent Acvr1 exon 2 with (i) a 5′ sequence of a human ACVR1 exon 2 wherein the 5′ sequence of the human ACVR1 exon 2 encodes human ACVR1 ectodomain amino acids comprising P at position 30, or (ii) a sequence modified from the 5′ sequence of the human ACVR1 exon 2 to include one or more silent mutations. In some embodiments, the modified rodent Acvr1 exon 4 encoding R206H differs from the endogenous rodent Acvr1 exon 4 by comprising a replacement of a sequence of the endogenous rodent Acvr1 exon 4 with a sequence of human ACVR1 exon 4 and a substitution of the codon for R at position 206 with a codon for H. In some embodiments, the modified rodent Acvr1 exon 6 differs from exon 6 of the endogenous rodent Acvr1 gene by comprising a substitution of the codon for S at position 330 with a codon for P and a synonymous nucleotide substitution.

In some embodiments, a modified rodent Acvr1 gene comprises: an endogenous rodent Acvr1 exon 1; a modified rodent Acvr1 exon 2 which differs from an endogenous rodent Acvr1 exon 2 by comprising a substitution of the codon for Q30 with a codon for P; an endogenous rodent Acvr1 exon 3; an endogenous rodent Acvr1 exon 4; a modified rodent Acvr1 exon 5 which differs from an endogenous rodent Acvr1 exon 5 by comprising a substitution of the codon for R258 with a codon for G; a modified rodent Acvr1 exon 6 that differs from exon 6 of the endogenous rodent Acvr1 gene by comprising a substitution of the codon for S330 with a codon for P; and endogenous rodent Acvr1 exons 7-9. In some embodiments, the modified rodent Acvr1 exon 2 differs from the endogenous rodent Acvr1 exon 2 by comprising a replacement of a 5′ sequence of the endogenous rodent Acvr1 exon 2 with (i) a 5′ sequence of a human ACVR1 exon 2 wherein the 5′ sequence of the human ACVR1 exon 2 encodes human ACVR1 amino acids comprising P at position 30, or (ii) a sequence modified from the 5′ sequence of the human ACVR1 exon 2 to include one or more silent mutations. In some embodiments, the modified rodent Acvr1 exon 6 differs from exon 6 of the endogenous rodent Acvr1 gene by comprising a substitution of the codon for S at position 330 with a codon for P and a synonymous nucleotide substitution.

In some embodiments, a modified rodent Acvr1 gene is in the germline genome of the rodent.

In some embodiments, a modified rodent Acvr1 gene is formed at an embryonic stage from an engineered Acvr1 gene in the rodent genome, wherein the engineered Acvr1 gene encodes an engineered Acvr1 polypeptide comprising the ectodomain of a human ACVR1 protein, and the transmembrane and cytoplasmic domains of the endogenous rodent Acvr1 protein except for the S330P substitution; and wherein the engineered Acvr1 gene comprises either (i) a human ACVR1 exon 4 in sense orientation flanked by a first pair of site-specific recombinase recognition sites (SRRS′), and a mutant rodent Acvr1 exon 4 encoding R206H in antisense orientation, flanked by a second pair of SRRS' that are different from the first pair of SRRS′, wherein the first and second pairs of SRRS' are oriented so that a recombinase can invert the mutant rodent Acvr1 exon 4 into sense orientation and delete the human ACVR1 exon 4 to form said modified rodent Acvr1 gene; or (ii) a human ACVR1 exon 5 in sense orientation flanked by a first pair of site-specific recombinase recognition sites (SRRS′), and a mutant rodent Acvr1 exon 5 encoding R258G in antisense orientation, flanked by a second pair of SRRS' that are different from the first pair of SRRS′, wherein the first and second pairs of SRRS' are oriented so that a recombinase can invert the mutant rodent Acvr1 exon 5 into sense orientation and delete the human ACVR1 exon 5 to form said modified rodent Acvr1 gene. In some embodiments, the recombinase is Cre. In some embodiments, the genome of the rodent comprises a polynucleotide encoding the recombinase under control of a Nanog promoter.

In some embodiments, a genetically modified rodent is heterozygous for a modified Acvr1 gene. In some embodiments, a genetically modified rodent is homozygous for a modified Acvr1 gene.

In some embodiments, a genetically modified rodent is a mouse or a rat.

In some embodiments, a genetically modified rodent survives at least 2-3 weeks after birth, and exhibits features characteristic of human FOP such as congenital toe malformations and/or injury-induced and idiopathic HO in post-natal life.

Also disclosed herein is an isolated tissue or cell of a genetically modified rodent described herein, wherein the isolated tissue or cell comprises a modified rodent Acvr1 gene. In some embodiments, the isolated cell is a sperm or an egg.

Further disclosed herein is a rodent embryonic stem (ES) cell, comprising a modified rodent Acvr1 gene at an endogenous rodent Acvr1 locus encoding a modified rodent Acvr1 polypeptide, wherein the modified rodent Acvr1 polypeptide comprises the ectodomain of a human ACVR1 protein, and the transmembrane and cytoplasmic domains of an endogenous rodent Acvr1 protein except for a S330P substitution and an FOP mutation selected from a R206H mutation or a R258G mutation; and wherein expression of the modified rodent Acvr1 gene is under control of the rodent Acvr1 promoter at the endogenous rodent Acvr1 locus.

In some embodiments, a rodent embryonic stem (ES) cell comprises a modified rodent Acvr1 gene at an endogenous rodent Acvr1 locus, wherein the modified rodent Acvr1 gene comprises: an endogenous rodent Acvr1 exon 1, a modified rodent Acvr1 exon 2 which differs from an endogenous rodent Acvr1 exon 2 by comprising a substitution of the codon for Q30 with a codon for P; an endogenous rodent Acvr1 exon 3; a modified rodent Acvr1 exon 4 which differs from an endogenous rodent Acvr1 exon 4 by comprising a substitution of the codon for R206 with a codon for H; an endogenous rodent Acvr1 exon 5; a modified rodent Acvr1 exon 6 that differs from exon 6 of the endogenous rodent Acvr1 gene by comprising a substitution of the codon for S330 with a codon for P; and endogenous rodent Acvr1 exons 7-9. In some embodiments, the modified rodent Acvr1 exon 2 differs from the endogenous rodent Acvr1 exon 2 by comprising a replacement of a 5′ sequence of the endogenous rodent Acvr1 exon 2 with (i) a 5′ sequence of a human ACVR1 exon 2 wherein the 5′ sequence of the human ACVR1 exon 2 encodes human ACVR1 amino acids comprising P at position 30, or (ii) a sequence modified from the 5′ sequence of the human ACVR1 exon 2 to include one or more silent mutations. In some embodiments, the modified rodent Acvr1 exon 4 encoding R206H differs from the endogenous rodent Acvr1 exon 4 by comprising a replacement of a sequence of the endogenous rodent Acvr1 exon 4 with a sequence of human ACVR1 exon 4 and a substitution of the codon for R at position 206 with a codon for H. In some embodiments, the modified rodent Acvr1 exon 6 differs from exon 6 of the endogenous rodent Acvr1 gene by comprising a substitution of the codon for S at position 330 with a codon for P and a synonymous nucleotide substitution.

In some embodiments, a rodent embryonic stem (ES) cell comprises a modified rodent Acvr1 gene at an endogenous rodent Acvr1 locus and under control of the endogenous Acvr1 promoter, wherein the modified rodent Acvr1 gene comprises: an endogenous rodent Acvr1 exon 1; a modified rodent Acvr1 exon 2 which differs from an endogenous rodent Acvr1 exon 2 by comprising a substitution of the codon for Q30 with a codon for P; an endogenous rodent Acvr1 exon 3; an endogenous rodent Acvr1 exon 4; a modified rodent Acvr1 exon 5 which differs from an endogenous rodent Acvr1 exon 5 by comprising a substitution of the codon for R258 with a codon for G; a modified rodent Acvr1 exon 6 that differs from exon 6 of the endogenous rodent Acvr1 gene by comprising a substitution of the codon for S330 with a codon for P; and endogenous rodent Acvr1 exons 7-9. In some embodiments, the modified rodent Acvr1 exon 2 differs from the endogenous rodent Acvr1 exon 2 by comprising a replacement of a 5′ sequence of the endogenous rodent Acvr1 exon 2 with (i) a 5′ sequence of a human ACVR1 exon 2 wherein the 5′ sequence of the human ACVR1 exon 2 encodes human ACVR1 amino acids comprising P at position 30, or (ii) a sequence modified from the 5′ sequence of the human ACVR1 exon 2 to include one or more silent mutations. In some embodiments, the modified rodent Acvr1 exon 6 differs from exon 6 of the endogenous rodent Acvr1 gene by comprising a substitution of the codon for S at position 330 with a codon for P and a synonymous nucleotide substitution.

Also disclosed herein is a rodent embryonic stem (ES) cell which comprises an engineered Acvr1 gene at an endogenous rodent Acvr1 locus and under control of the endogenous Acvr1 promoter, wherein the engineered Acvr1 gene encodes an engineered Acvr1 polypeptide comprising the ectodomain of a human ACVR1 protein, and the transmembrane and cytoplasmic domains of the endogenous rodent Acvr1 protein except for a S330P substitution; and wherein the engineered Acvr1 gene comprises: (i) a human ACVR1 exon 4 in sense orientation flanked by a first pair of site-specific recombinase recognition sites (SRRS′), and a mutant rodent Acvr1 exon 4 encoding R206H in antisense orientation, flanked by a second pair of SRRS' that are different from the first pair of SRRS′, wherein the first and second pairs of SRRS' are oriented so that a recombinase can invert the mutant rodent Acvr1 exon 4 into sense orientation and delete the human ACVR1 exon 4 to form a modified rodent Acvr1 gene; or (ii) a human ACVR1 exon 5 in sense orientation flanked by a first pair of site-specific recombinase recognition sites (SRRS′), and a mutant rodent Acvr1 exon 5 encoding R258G in antisense orientation, flanked by a second pair of SRRS' that are different from the first pair of SRRS′, wherein the first and second pairs of SRRS' are oriented so that a recombinase can invert the mutant rodent Acvr1 exon 5 into sense orientation and delete the human ACVR1 exon 5 to form a modified rodent Acvr1 gene.

In some embodiments, a rodent ES cell is a mouse ES cell or a rat ES cell.

Also disclosed herein is a rodent embryo comprising a rodent ES cell disclosed herein comprising a modified Acvr1 gene or an engineered Acvr1 gene.

Disclosed herein is a targeting nucleic acid construct, comprising a modified rodent Acvr1 gene sequence, flanked by a 5′ homology arm and a 3′ homology arm, wherein the modified rodent Acvr1 gene sequence comprises: a modified rodent Acvr1 exon 2 which differs from a wild-type rodent Acvr1 exon 2 by comprising a substitution of the codon for Q30 with a codon for P; a wild-type rodent Acvr1 exon 3; a modified rodent Acvr1 exon 4 which differs from a wild-type rodent Acvr1 exon 4 by comprising a substitution of the codon for R206 with a codon for H; a wild-type rodent Acvr1 exon 5; and a modified rodent Acvr1 exon 6 that differs from a wild-type rodent Acvr1 exon 6 by comprising a substitution of the codon for S330 with a codon for P; wherein the 5′ homology arm and the 3′ homology arm are substantially identical to the sequences at a rodent Acvr1 gene locus to mediate integration of the modified rodent Acvr1 gene sequence into the rodent Acvr1 gene.

Also disclosed herein is a targeting nucleic acid construct, comprising a modified rodent Acvr1 gene sequence, flanked by a 5′ homology arm and a 3′ homology arm, wherein the modified rodent Acvr1 gene sequence comprises: a modified rodent Acvr1 exon 2 which differs from a wild type rodent Acvr1 exon 2 by comprising a substitution of the codon for Q30 with a codon for P; a wild type rodent Acvr1 exon 3; a wild type rodent Acvr1 exon 4; a modified rodent Acvr1 exon 5 which differs from a wild type rodent Acvr1 exon 5 by comprising a substitution of the codon for R258 with a codon for G; a modified rodent Acvr1 exon 6 that differs from a wild-type rodent Acvr1 exon 6 by comprising a substitution of the codon for S330 with a codon for P; and wherein the 5′ homology arm and the 3′ homology arm are substantially identical to the sequences at a rodent Acvr1 gene locus to mediate integration of the modified rodent Acvr1 gene sequence into the rodent Acvr1 gene.

Further disclosed herein is a targeting nucleic acid construct, which comprises an engineered Acvr1 gene sequence, flanked by a 5′ homology arm and a 3′ homology arm, wherein the engineered Acvr1 gene sequence comprises: a modified rodent Acvr1 exon 2 which differs from a wild-type rodent Acvr1 exon 2 by comprising a substitution of the codon for Q30 with a codon for P; a wild-type rodent Acvr1 exon 3; a human ACVR1 exon 4 in sense orientation flanked by a first pair of site-specific recombinase recognition sites (SRRS′), and a mutant rodent Acvr1 exon 4 encoding R206H in antisense orientation, flanked by a second pair of SRRS' that are different from the first pair of SRRS′, wherein the first and second pairs of SRRS' are oriented so that a recombinase can invert the mutant rodent Acvr1 exon 4 into sense orientation and delete the human ACVR1 exon 4; a wild-type rodent Acvr1 exon 5; and a modified rodent Acvr1 exon 6 that differs from a wild-type rodent Acvr1 exon 6 by comprising a substitution of the codon for S330 with a codon for P; wherein the 5′ homology arm and the 3′ homology arm are substantially identical to the sequences at a rodent Acvr1 gene locus to mediate integration of the engineered Acvr1 gene sequence into the rodent Acvr1 gene.

Also disclosed herein is a targeting nucleic acid construct which comprises a modified rodent Acvr1 gene sequence, flanked by a 5′ homology arm and a 3′ homology arm, wherein the modified rodent Acvr1 gene sequence comprises: a modified rodent Acvr1 exon 2 which differs from a wild type rodent Acvr1 exon 2 by comprising a substitution of the codon for Q30 with a codon for P; a wild type rodent Acvr1 exon 3; a wild type rodent Acvr1 exon 4; a human ACVR1 exon 5 in sense orientation flanked by a first pair of site-specific recombinase recognition sites (SRRS′), and a mutant rodent Acvr1 exon 5 encoding R258G in antisense orientation, flanked by a second pair of SRRS' that are different from the first pair of SRRS′, wherein the first and second pairs of SRRS' are oriented so that a recombinase can invert the mutant rodent Acvr1 exon 5 into sense orientation and delete the human ACVR1 exon 5; a modified rodent Acvr1 exon 6 that differs from a wild-type rodent Acvr1 exon 6 by comprising a substitution of the codon for S330 with a codon for P; and wherein the 5′ homology arm and the 3′ homology arm are substantially identical to the sequences at a rodent Acvr1 gene locus to mediate integration of the engineered Acvr1 gene sequence into the rodent Acvr1 gene.

Disclosed herein is a method of making a genetically modified rodent, comprising modifying the rodent genome to comprise a modified rodent Acvr1 gene at an endogenous rodent Acvr1 locus, wherein the modified rodent Acvr1 gene encodes a modified rodent Acvr1 polypeptide, wherein the modified rodent Acvr1 polypeptide comprises the ectodomain of a human ACVR1 protein, and the transmembrane and cytoplasmic domains of an endogenous rodent Acvr1 protein except for a S330P substitution and an FOP mutation selected from a R206H mutation or a R258G mutation; and wherein expression of the modified rodent Acvr1 gene is under control of the rodent Acvr1 promoter at the endogenous rodent Acvr1 locus.

In some embodiments, the rodent genome is modified by modifying the genome of a rodent ES cell to comprise a modified rodent Acvr1 gene at the endogenous rodent Acvr1 locus of the rodent ES cell, thereby obtaining a genetically modified ES cell, and generating a rodent from the obtained genetically modified ES cell. In some embodiments, the genome of the rodent ES cell is modified by introducing a targeting nucleic acid construct described herein which comprises a modified rodent Acvr1 gene sequence, flanked by a 5′ homology arm and a 3′ homology arm.

Also disclosed herein is a method of making a genetically modified rodent, comprising modifying a rodent genome to comprise an engineered Acvr1 gene at an endogenous rodent Acvr1 locus, wherein the engineered Acvr1 gene encodes an engineered Acvr1 polypeptide comprising the ectodomain of a human ACVR1 protein, and the transmembrane and cytoplasmic domains of the endogenous rodent Acvr1 protein except for the S330P substitution; and wherein the engineered Acvr1 gene comprises: (i) a human ACVR1 exon 4 in sense orientation flanked by a first pair of site-specific recombinase recognition sites (SRRS′), and a mutant rodent Acvr1 exon 4 encoding R206H in antisense orientation, flanked by a second pair of SRRS' that are different from the first pair of SRRS′, wherein the first and second pairs of SRRS' are oriented so that a recombinase can invert the mutant rodent Acvr1 exon 4 into sense orientation and delete the human ACVR1 exon 4 to form a modified rodent Acvr1 gene; or (ii) a human ACVR1 exon 5 in sense orientation flanked by a first pair of site-specific recombinase recognition sites (SRRS′), and a mutant rodent Acvr1 exon 5 encoding R258G in antisense orientation, flanked by a second pair of SRRS' that are different from the first pair of SRRS′, wherein the first and second pairs of SRRS' are oriented so that a recombinase can invert the mutant rodent Acvr1 exon 5 into sense orientation and delete the human ACVR1 exon 5 to form a modified rodent Acvr1 gene.

In some embodiments, the rodent genome is modified by modifying the genome of a rodent ES cell to comprise said engineered Acvr1 gene at the endogenous rodent Acvr1 locus of the rodent ES cell, thereby obtaining a genetically modified ES cell; and generating a rodent from the obtained genetically modified ES cell. In some embodiments, the genome of the rodent ES cell is modified by introducing a targeting nucleic acid construct described herein that comprises an engineered Acvr1 gene sequence.

In some embodiments, wherein the recombinase is Cre.

In some embodiments, the genome of the rodent comprises a polynucleotide encoding the recombinase under control of a Nanog promoter, and wherein the recombinase acts at an embryonic stage of the rodent to invert the mutant rodent Acvr1 exon into sense orientation and delete the wild-type Acvr1 exon thereby forming a modified rodent Acvr1 gene encoding a modified Acvr1 polypeptide comprising the ectodomain of a human ACVR1 protein, and the transmembrane and cytoplasmic domains of the endogenous rodent Acvr1 protein except for the S330P substitution and the FOP mutation.

In some embodiments of a method described herein, the rodent is a mouse or a rat.

Further disclosed herein is a method of testing a candidate therapeutic compound for treating ectopic bone formation, which comprises providing a genetically modified rodent described herein, administering the candidate compound to the rodent; and determining whether the candidate compound inhibits the development of ectopic bone formation in the rodent.

### BRIEF DESCRIPTION OF THE DRAWINGS

The file of this patent or application contains at least one drawing executed in color. Copies of this patent with color drawing(s) will be provided by the Patent and Trademark Office upon request and payment of the necessary fee.

FIG. 1A depicts the genomic structure of the wild-type (unmodified) mouse Acvr1 locus. The exons are depicted by vertical bars above the line which represents mouse genomic DNA. Positions of amino acids Q30, R206 and S330 within the respective coding exons are indicated. Positions of the primers used in the TaqMan assays (7340mTD2, Acvri5U/D, and 8431mAS.WT) are also indicated.

FIG. 1B depicts a targeting nucleic acid construct for generating the 8431 allele, which is an engineered mouse Acvr1 allele having Q30P and S330P humanization, with reversed FOP COIN allele (R206H), and containing Neo and Hygro resistance cassettes. The nucleotide sequence of the first 78 bp of mouse Acvr1 coding exon 2 was replaced with the corresponding human ACVR1 sequence, with 6 silent mutations and Q (CAG) to P (CCC) coding for human amino acid 30. The rest of exon 2 remains mouse. A 45 bp deletion was made in mouse intron 2 for a loss-of-allele Taqman assay and to insert a Prm-Dre hUb-Hygro cassette. Following the cassette, the remainder of intron 2 was not changed. In intron 3, a loxP-3′ human ACVR1 intron 3-human coding exon 4-5′ human intron 4 (336 bp total)-lox2372 sequence was inserted. Following the lox2372 was a reverse oriented sequence consisting of mouse intron 4-mouse exon 4 with R206H FOP mutation-mouse intron 3 (328 bp total). This was followed by a loxP site in reverse orientation relative to the 5′ site, a rabbit HBB2 splice acceptor sequence in reverse orientation, and a lox2372 site, in reverse relative to its 5′ counterpart. This was followed by additional mouse intron 4 sequence (in forward orientation), then a Frt-hUb-Neo-Frt cassette. In mouse coding exon 6, a synonymous G to C point mutation was made, along with a S330P (TCC to CCC) humanizing change. The sizes of the various fragments in the allele are as follows: RoxP-mPrm1-Drei-pA-hUb1-em7-Hygro-pA-RoxP cassette (4,976 bp) in mouse intron 2; Frt-hUb-Neo-pA-Frt in intron 4 (2,605 bp); 78 bp mouse sequence replaced by 78 bp of human sequence, coding exon 2; 336 bp intron 3-ex4-int4 human sequence with inversion of corresponding mouse sequence (328 bp). In exon 6, a single T to C change to create S330P humanization. The constituents and sequences of the various DNA fragments in the 8431 allele identified as “A”, “B”, “C”, “D”, “E”, “F”, and “G” are set forth in FIG. 1C.

FIG. 1C describes the constituents and sequences of various DNA fragments in the 8431 allele identified as “A”, “B”, “C”, “D”, “E”, “F”, and “G” in FIG. 1B.

FIG. 1D depicts the engineered Acvr1 allele after the Neo and Hygro resistance cassettes in the 8431 allele were deleted by FlpO and Dre recombinases, respectively. The resulting allele is designated as the “8432 allele”. After cassette deletion, RoxP and cloning sites (76 bp) remain inserted in mouse intron 2. Frt and cloning sites (59 bp) remain inserted in mouse intron 4. The constituents and sequences of the various DNA fragments in the 8432 allele identified as “H” and “I” are set forth in FIG. 1E.

FIG. 1E sets forth the constituents and sequences of the DNA fragments in the 8432 allele identified as H″ and “I”.

FIG. 1F depicts the 8955 Allele, derived from the 8431 or 8432 allele after Cre activation which deletes the human intron 3-exon 4-intron 4 sequence and places corresponding mouse sequence (328 bp) in the correct orientation. Briefly, in intron 3 of either the 8431 or 8432 allele, Cre-mediated deletion flips the sequence between loxP sites, resulting in lox2372 sites that face in the same direction; Cre then deletes the sequence between the lox2372 sites, leaving a single lox2372 site and a single loxP. Alternatively, Cre flips the sequence between the lox2372 sites in intron 3 of either the 8431 or 8432 allele and then delete sequence between same-facing loxP sites-the end result is the same sequence. As a result of the action by Cre, the human ACVR1 intron 3-human exon 4-5′ human intron 4 fragment in either the 8431 or 8432 allele is removed and the corresponding mouse sequence is inverted to create mouse intron 3-mouse exon 4 with R206H FOP mutation-mouse intron 4, ready for transcription. If the 8341 allele is acted by Cre, the Neo and Hyg cassettes can be subsequently deleted, RoxP and cloning sites (76 bp) remain inserted in mouse intron 2; and Frt and cloning sites (59 bp) remain inserted in mouse intron 4. The remaining parts of the 8955 allele are the same as the 8432 allele: the first 78 bp of mouse Acvr1 exon 2 is replaced with the corresponding human ACVR1 sequence, with 6 silent mutations and Q (CAG) to P (CCC) human amino acid 30; the rest of exon 2 remains mouse; a 45 bp deletion was made in mouse intron 2 for a loss-of-allele Taqman assay; and RoxP and cloning sites (76 bp) remain inserted in mouse intron 2; in mouse coding exon 6, a synonymous G to C point mutation was made, along with a S330P (TCC to CCC) humanizing change. The constituents and sequence of the DNA fragment labeled as “J” is set forth in FIG. 1G.

FIGS. 2A-2B set forth an alignment of the protein sequences of mouse Acvr1 (SEQ ID NO: 1), human ACVR1 (SEQ ID NO: 3) and an Acvr1 protein having Q30P and S330P (SEQ ID NO: 5) encoded by the engineered 8431 or 8432 allele, which are engineered alleles that includes a wild-type exon 4 of a human ACVR1 gene in sense orientation and a mutant mouse exon 4 encoding R206H in anti-sense orientation. The signal peptide, transmembrane domain, and protein kinase domain are also indicated.

FIGS. 2C-2D set forth an alignment of the protein sequences of mouse Acvr1 (SEQ ID NO: 1), human ACVR1 (SEQ ID NO: 3), and an Acvr1 protein having Q30P, R206H and S330P (SEQ ID NO: 7) encoded by the 8955 allele (a modified Acvr1 allele which includes a mutant mouse exon 4 encoding R206H in sense orientation). The signal peptide, transmembrane domain, and protein kinase domain are also indicated.

FIG. 3A sets forth the sequence of a mouse Acvr1 protein (SEQ ID NO: 1).

FIG. 3B sets forth the sequence of a mouse Acvr1 coding sequence (SEQ ID NO: 2).

FIG. 3C sets forth the sequence of a human ACVR1 protein (SEQ ID NO: 3).

FIG. 3D sets forth the sequence of a human ACVR1 coding sequence (SEQ ID NO: 4).

FIG. 3E sets forth the sequence of an engineered Acvr1 protein containing Q30P and S330P humanized amino acids (SEQ ID NO: 5).

FIG. 3F sets forth the coding sequence (SEQ ID NO: 6) of an engineered Acvr1 allele (8431 or 8432) that encodes the engineered Acvr1 protein of SEQ ID NO: 5 containing Q30P and S330P humanized amino acids.

FIG. 3G sets forth the sequence of a modified Acvr1 protein containing Q30P and S330P humanized amino acids, as well as R206H (SEQ ID NO: 7).

FIG. 3H sets forth the coding sequence (SEQ ID NO: 8) of an engineered Acvr1 allele (8955) that encodes the modified Acvr1 protein of SEQ ID NO: 7 containing Q30P, R206H, and S330P.

FIGS. 4A-4D set forth the sequence of the 8431 Allele (SEQ ID NO: 9): mouse intron (lower case)—(in parenthesis: human coding exon 2, synonymous changes (underlined), CAG to CCC Q30P)—mouse exon 2 (bold)—mouse intron 2 (lower case)—XhoI (underlined)—RoxP (italics bold)—Protamine promoter (underlined)—Dre ORF (bold, intron in lower case)—poly(A) (italics)—hUb (underlined)—Em7 (bold)—Hygro (bold underlined)—poly(A) (bold italics)—RoxP (bold italics)—Iceu1 (underlined)—NheI (bold)—mouse intron 2 (lower case)—mouse exon 3 (underlined)—mouse intron 3 (lower case)—AgeI (bold)—loxP (bold underlined)—(in parenthesis: human intron 3 (lower case)—human exon 4 (underlined)—human intron 4 (lower case))—MluI (bold)—lox2372 (bold underlined)—[[in double bracket: mouse intron 4 (lower case)—mouse exon 4 (bold), R206H (bold underlined)—mouse intron 3-KpnI (bold)—loxP (underlined)—rabbit HBB2 splice acceptor (underlined italics bold)—Lox2372 (underlined)]] (region in double bracket is reverse complemented compared to the rest of the sequence)—BamHI (bold)—mouse intron 4 (lower case)—XhoI (bold)—Frt (bold underlined)—hUb (underlined)—Em7 (bold)—Neo (bold underlined)—poly(A) (italics)—Frt (italics bold underlined)—NheI (bold)—mouse intron 4 (lower case)—mouse exon 5 (bold)—mouse intron 5 (lower case)—mouse exon 6 (bold), silent mutation (underlined) and TCC to CCC (S330P) (underlined).

FIGS. 5A-5C set forth the sequence of the 8432 Allele (SEQ ID NO: 10): mouse intron (lower case—(in parenthesis: human coding exon 2, synonymous changes underlined, CAG to CCC Q30P)—mouse exon 2 (bold)—mouse intron 2 (lower case)—XhoI (underlined)—RoxP (italics bold)—Iceu1 (underlined)—NheI (bold)—mouse intron 2 (lower case)—mouse exon 3 (underlined)—mouse intron 3 (lower case)—AgeI (bold)—loxP (bold underlined)—(in parenthesis: human intron 3 (lower case)—human exon 4 (underlined)—human intron 4 (lower case)—MluI (bold)—lox2372 (bold underlined))—[[in double bracket: mouse intron 4 (lower case)—mouse exon 4 (bold), R206H (bold underlined)—mouse intron 3-KpnI (bold)—loxP (underlined)—rabbit HBB2 splice acceptor (italics bold underlined)—Lox2372 (underlined)]] (region in double bracket is reverse complemented compared to the rest of the sequence)—BamHI (bold)—mouse intron 4 (lower case)—XhoI (bold)—Frt (bold italics underlined)—NheI (bold)—mouse intron 4 (lower case)—mouse exon 5 (bold)—mouse intron 5 (lower case)—mouse exon 6 (bold), silent mutation (underlined) and TCC to CCC (S330P) (underlined).

FIGS. 6A-6C set forth the sequence of the 8955 Allele (SEQ ID NO: 11): mouse intron (lower case)—(in parenthesis: human coding exon 2, synonymous changes (underlined), CAG to CCC Q30P)—mouse exon 2-mouse intron 2 (lower case)—XhoI (underlined)—RoxP-Iceu1 (underlined)—NheI-mouse intron 2 (lower case)—mouse exon 3 (underlined)—mouse intron 3 (lower case)—AgeI-loxP-KpnI-mouse intron 3 (lower case)—mouse exon 4, R206H codon underlined-mouse intron 4 (lower case)—lox2372 (underlined)—BamHI-mouse intron 4 (lower case)—XhoI-Frt (underlined)—NheI-mouse intron 4 (lower case)—mouse exon 5-mouse intron 5 (lower case)—mouse exon 6, silent mutation (underlined) and TCC to CCC (S330P) (underlined).

FIGS. 7A-7C demonstrate the phenotypes of Acvr1^{huecto[R206H]FIEx;[S330P]/+}; Nanog-Cre mice. FIG. 7A: Acvr1^{huecto[R206H]FIEx;[S330P]/+}; Nanog-Cre mice exhibited persistent interdigital webbing between digits 2-4 (arrow in the bright field panel) and truncation of hindlimb digits 1 and 5 (asterisk). The apparent intra-digit fusion (arrow in the μCT panel) is likely an artifact of low μCT resolution. FIG. 7B: Acvr1^{huecto[R206H]FIEx;[S330P]/+}; Nanog-Cre mice exhibited FOP-like “spontaneous” HO. 15 of 37 Acvr1^{huecto[R206H]FIEx;[S330P]/+}; Nanog-Cre mice were μCT′d at 4-6 wks of age; 11 of the 15 mice (74%) had one or more sites of “spontaneous” HO; 6 of 15 (40%) had HO ankylosing the mandible (dashed arrow); 5 of 15 (30%) had posterior knee region HO (non-ankylosing intramuscular example is indicated by an arrow); 2 of 15 (13%) had ankle region HO. FIG. 7C: Acvr1^{huecto[R206H]FIEx;[S330P]/+}; Nanog-Cre mice (lower curve) exhibited reduced survival as compared to wild-type mice (upper line), likely due to jaw ankylosing HO.

FIG. 8 shows that an anti-Activin A blocking antibody inhibited HO formation and promoted survival in FOP mice. Wild type mice and Acvr1^{huecto[R206H]FIEx;[S330P]/+}; Nanog-Cre mice (“FOP mice”) were treated with an anti-Activin A monoclonal antibody (Garetosmab) or an isotype control antibody. When the Garetosmab treatment was initiated at 2 weeks of age, FOP mice exhibited 100% survival through 8 weeks of age (n=15). In contrast, FOP mice exhibited a median survival of ˜6 weeks in the absence of Garetosmab (n=14).

FIG. 9 shows that the S330P mutation made ACVR1 less responsive to ligand and antibody activation. Acvr1^{[R206H]/+} and Acvr1^{huecto[R206H]FIEx;[S330P]/+} mES cells were treated with Activin A and BMP7 for 1 hr (after 1 hr starvation). In-cell ELISA was performed with cell lysates to measure P-Smad1 and Total Smad1 levels. The ratio of P-Smad1/T-Smad1 was calculated and plotted against the ligand concentration. Cell lysates were also run on the Western blots to compare the P-Smad1/5/8 levels of Acvr1^{[R206H]/+} and Acvr1^{huecto, S330P[R206H]/+} mES cells treated with Activin A, BMP2, BMP7, and BMP10 for 1 hr. Cyclophilin B was used as a loading control in the immunoblot. Both in-cell ELIS and immunoblot data show that the S330P mutation made ACVR1 less responsive to ligand activation, which is indicated by significantly decreased P-Smad1/T-Smad1 levels in ELIS assay and P-Smad1/5 levels in the Western blot of Acvr1^{huecto[R206H]FIEx;[S330P]/+} mES cells treated with ligands compared to the ones of Acvr1^{[R206H]+} mES cells.

FIG. 10 shows that mouse Acvr1 kinase domain is more active than human Acvr1 kinase domain. N-terminally His tagged human ACVR1 (“hACVR1”), hACVR1[R206H], mouse ACVR1 (“mACVR1”), and mACVR1[R206H] were expressed in ExpiCHO cells and purified (Ni-column followed by size exclusion chromatography-SEC). The kinase activity (ability to phosphorylate casein as a substrate) of the purified human and mouse ACVR1 kinases was compared at room temperature (“RT”). In this experiment, a fixed amount of casein substrate and a fixed amount of ATP were incubated with different amounts of purified human and mouse ACVR1 kinases. After unused ATP was depleted, the produced ADP was re-converted to ATP, which was converted into light by luciferase. The luciferase light generated was correlated with the amount of ADP generated in the kinase assay, which is indicative of kinase activity.

### DETAILED DESCRIPTION

Fibrodysplasia ossificans progressiva (FOP) is a particularly rare and exceedingly disabling genetic disease in which heterotopic ossification (HO) results in joint ankylosis and destruction of skeletal muscle and its associated soft tissues. Approximately 95% of FOP is caused by the R206H mutation in activin A type I receptor (Acvr1). In juxtaposition to the devastatingly disabling consequences of HO during post-natal life, the developmental malformations associated with FOP are comparatively benign, the most overt of which being a truncating malformation of the great toe. However, despite mouse and human ACVR1 proteins sharing about 98% sequence identity, Acvr1^{R206H/+} mice die perinatally. A “conditional-on” mouse model of FOP was generated (Acvr1^{[R206H]FIEx/+}) and described in U.S. Pat. No. 9,510,569 B1 (Regeneron Pharmaceuticals), where a COIN Acvr1 allele was designed such that the mouse expressed a wild-type Acvr1 gene until after the mouse was induced to flip a R206H-encoding mutant exon 4 into sense orientation, delete the wild-type exon 4, and express a mutant Acvr1 comprising the R206H mutation. Although this conditional-on mouse model has been used successfully to discover the key molecular and cellular mechanism that drives HO in FOP, when the Acvr1^{[R206H]FIEx/+} model is recombined early in development using Nanog-Cre, the resulting Acvr1^{R206H/+}; Nanog-Cre mice display both neonatal lethality and skeletal deformities that are substantially more severe than those observed in humans with FOP. It has been surprisingly found in accordance with this disclosure that humanizing the mouse Acvr1 protein comprising the R206H mutation by substituting the mouse ectodomain with the human ectodomain and substituting Serine 330 with a Proline, as is found in human ACVR1, alleviated neonatal lethality. Additionally, the resultant mice exhibited congenital toe malformations and developed injury-induced and idiopathic HO in post-natal life, closely recapitulating human FOP. Hence, provided herein are genetically modified rodent animals suitable for use as a rodent model of FOP.

### Activin A Receptor Type 1 (ACVR1)

ACVR1 is highly conserved across species. The human ACVR1 gene is located on chromosome 2, is about 139 kb in length, and includes 9 coding exons encoding a polypeptide of 509 amino acids. The mouse Acvr1 gene is located on chromosome 2, is about 120 kb in length, and also includes 9 coding exons encoding a polypeptide of 509 amino acids.

Both human, mouse and rat Acvr1 genes have 5′ non-coding exons and 9 coding exons. For simplicity, the numbering of the exons herein refers to the coding exons of an Acvr1 gene. For example, exon 1 of an Acvr1 gene refers to the first coding exon of the Acvr1 gene.

Unless specified otherwise, references to rodent Acvr1 gene, endogenous rodent Acvr1 gene, rodent Acvr1 exon, an endogenous rodent Acvr1 exon, rodent Acvr1 polypeptide, and endogenous rodent Acvr1 polypeptide, all refer to wild-type rodent Acvr1 sequences; and references to human ACVR1 gene, human ACVR1 exon, and human ACVR1 protein, all refer to wild-type human sequences.

Exemplary Acvr1 mRNA and protein sequences from human, mouse and rat are available in GenBank under the following accession numbers and are also set forth in the Sequence Listing.

TABLE 1

| SEQ | | |
|---|---|---|
| ID NO | Description | Features |
| 1 | Mus musculus Acvr1 | Length: 509 aa |
| | protein, NP_001341978.1 | Signal peptide: 1-20 |
| | | Ectodomain: 21-123 |
| | | Transmembrane domain: 124-146 |
| | | Cytoplasmic domain: 147-509 |
| 2 | Mus musculus Acvr1 | Length: 1530 bp |
| | mRNA, CDS | |
| 3 | Homo sapiens ACVR1 | Length: 509 aa |
| | protein, NP_001096.1 | Signal peptide: 1-20 |
| | | Ectodomain: 21-123 |
| | | Transmembrane domain: 124-146 |
| | | Cytoplasmic domain: 147-509 |
| 4 | Homo sapiens ACVR1 | Length: 1530 bp |
| | mRNA, CDS | |
| 41 | Rattus norvegicus Acvr1 | Length: 509 aa |
| | protein, NP_077812.1 | |
| 42 | Rattus norvegicus Acvr1 | Length: 1780 bp |
| | mRNA, NM_024486.1 | CDS: 148 . . . 1677 (1530 bp) |

In some embodiments, a full length human ACVR1 protein is represented by the amino acid sequence as set forth in SEQ ID NO: 3. In some embodiments, a human ACVR1 protein may be represented by an amino acid sequence that is substantially identical to the amino acid sequence set forth in SEQ ID NO: 3.

In some embodiments, a full length mouse Acvr1 protein is represented by the amino acid sequence as set forth in SEQ ID NO: 1. In some embodiments, a mouse Acvr1 protein may be represented by an amino acid sequence that is substantially identical to the amino acid sequence set forth in SEQ ID NO: 1.

In some embodiments, a full length rat Acvr1 protein is represented by the amino acid sequence as set forth in SEQ ID NO: 40. In some embodiments, a rat Acvr1 protein may be represented by an amino acid sequence that is substantially identical to the amino acid sequence set forth in SEQ ID NO: 41.

In referring to a given sequence as being “substantially identical” to a reference sequence, it includes embodiments where the given sequence is at least 98% identical, at least 98.5%, at least 99% identical, or at least 99.5% identical, to a reference sequence; for example, a given amino acid sequence that is at least substantially identical to a reference sequence may differ from the reference sequence by 1, 2, or 3, amino acids, or may differ by not more than 3, 2, or 1 amino acid(s), which may be a result of naturally occurring polymorphism, for example.

### Modified ACVR1 Genes and Polypeptides

References to a “modified Acvr1 gene”, as used herein, are meant to include Acvr1 genes comprising or resulting from a modification (e.g., a mutation) to an endogenous or a wild-type Acvr1 gene, such as an endogenous or wild-type rodent (e.g., mouse or rat) Acvr1 gene. A modification can include addition, deletion, or substitution of one or more nucleotides made to an endogenous or a wild-type Acvr1 gene. In some embodiments, a modification is a substitution of one or more nucleotides in an endogenous or a wild-type Acvr1 gene. In some embodiments, a modification is a substitution of a contiguous sequence of nucleotides in an endogenous or a wild-type Acvr1 gene, e.g., a replacement of a contiguous sequence of nucleotides in a rodent (e.g., mouse or rat) Acvr1 gene with a corresponding sequence of a human ACVR1 gene. In some embodiments, a modification is a deletion of one or more nucleotides in an endogenous or a wild-type Acvr1 gene. In some embodiments, a modification to an endogenous or a wild-type Acvr1 gene is a silent mutation, i.e., the modification does not change the amino acid sequence encoded by the endogenous or wild-type Acvr1 gene. In some embodiments, a modification to an endogenous or a wild-type Acvr1 gene results in an addition, deletion, or substitution of one or more amino acids in the encoded protein, thereby providing a modified or mutant Acvr1 protein. In some embodiments, a modification to an endogenous or a wild-type Acvr1 gene results in substitution of an amino acid in the Acvr1 protein. In some embodiments, a modification to an endogenous or a wild-type rodent Acvr1 gene (e.g., a mouse or rat Acvr1 gene) results in substitution of an amino acid in the rodent Acvr1 protein with an amino acid found at the corresponding position in a human ACVR1 protein.

In some embodiments, a modified Acvr1 gene is a modified rodent (e.g., mouse or rat) Acvr1 gene, where a modification to a rodent Acvr1 gene (i.e., an endogenous or wild-type rodent Acvr1 gene) is made. In some embodiments, a modification to a rodent Acvr1 gene comprises substitution of one or more nucleotides in the coding sequence for the ectodomain of the rodent Acvr1 protein to code for the ectodomain of a human ACVR1 protein. In some embodiments, a modification to a rodent Acvr1 gene comprises replacement of the coding sequence for the entire ectodomain of a rodent Acvr1 protein with a coding sequence for the entire ectodomain of a human ACVR1 protein. Because of the high degree of sequence identity across species, it is not always necessary to replace the coding sequence for the entire ectodomain of a rodent Acvr1 protein in order for the modified Acvr1 gene to code for the ectodomain of a human ACVR1 protein. For example, the ectodomains of human and mouse Acvr1 proteins differ only at amino acid at position 30, with Gln (Q) for the mouse Acvr1 protein and Pro (P) for the human ACVR1 protein. Thus, modification to a mouse Acvr1 gene to substitute one or more nucleotides in the codon for Q30 to code for Pro instead would result in a modified mouse Acvr1 gene encoding a modified mouse Acvr1 protein having the ectodomain of a human ACVR1 protein.

In some embodiments, a modification to a rodent Acvr1 gene comprises substitution of one or more nucleotides in the coding sequence for the ectodomain of a rodent Acvr1 protein such that the resulting modified rodent Acvr1 gene encodes the entire ectodomain of a human ACVR1 protein. In some embodiments, a modification is made to a mouse Acvr1 gene, which modification comprises substitution of one or more nucleotides in the codon for amino acid Glutamine at position 30 (Q30) to code for Proline instead, resulting in a modified mouse Acvr1 gene which encodes the entire ectodomain of a human ACVR1 protein.

In some embodiments, a modification to a rodent Acvr1 gene comprises replacement of a contiguous sequence coding for amino acids within the ectodomain of a rodent Acvr1 protein such that the resulting modified rodent Acvr1 gene encodes the entire ectodomain of a human ACVR1 protein. In some embodiments, a modification is made to a mouse Acvr1 gene, which modification comprises a replacement of a contiguous nucleic acid sequence in exon 2 of the mouse Acvr1 gene coding for amino acids surrounding and including Q30, with a contiguous nucleic acid sequence in exon 2 of a human ACVR1 gene coding for the corresponding amino acids of the human ACVR1 protein. In some embodiments, the contiguous nucleic acid sequence in exon 2 of a mouse Acvr1 gene that is being replaced encodes about 5-45 amino acids including Q30, or about 10-40 amino acids including Q30, or about 20-35 amino acids including Q30, or about 25-35 amino acids including Q30. In some embodiments, the contiguous nucleic acid sequence in exon 2 that is being replaced encodes the amino acid sequence of EKPKVNQKLYMCVCEGLSCGNEDHCE (SEQ ID NO: 40) (Q in this sequence representing Q30).

In some embodiments, a modification to a rodent Acvr1 gene comprises substitution of one or more nucleotides in the codon for amino acid Serine at position 330 (S330) to code for Proline instead, which is the amino acid found at position 330 of a human ACVR1 protein. Amino acid 330 is in the cytoplasmic domain, and the codon for amino acid 330 is in exon 6 for both human and rodent (e.g., mouse) Acvr1 genes. In some embodiments, a modification to a rodent Acvr1 gene comprises a replacement of a contiguous nucleic acid sequence in exon 6 of the rodent Acvr1 gene coding for amino acids surround and including S330 of a rodent Acvr1 protein, with a contiguous nucleic acid sequence in exon 6 of a human ACVR1 gene coding for the corresponding amino acids (including P330) of the human ACVR1 protein. In some embodiments, the contiguous nucleic acid sequence in exon 6 of a rodent Acvr1 gene that is being replaced encodes about 5-45 amino acids including S330.

As used herein, the phrase “corresponding to” or grammatical variations thereof when used in the context of the numbering of positions in a given polypeptide or nucleic acid molecule refers to the numbering of a specified reference polypeptide or nucleic acid molecule when the given amino acid or nucleic acid molecule is compared to the reference molecule. In other words, the position of an amino acid residue or nucleotide in a given polymer is designated with respect to the reference molecule rather than by the actual numerical position of the amino acid residue or nucleotide within the given polymer. For example, a given amino acid sequence can be aligned to a reference sequence by introducing gaps to optimize residue matches between the two sequences. In these cases, although the gaps are present, the numbering of the residue in the given amino acid or nucleic acid sequence is made with respect to the reference sequence to which it has been aligned. For example, when the human and mouse Acvr1 proteins are aligned, the Proline at position 30 in the human ACVR1 protein is considered to correspond to Glutamine at position 30 in the mouse Acvr1 protein; and the Proline at position 330 in the human ACVR1 protein is considered to correspond to Serine at position 330 in the mouse Acvr1 protein.

In some embodiments, a rodent Acvr1 gene has been modified to comprise a combination of modifications described above. In some embodiments, a rodent Acvr1 gene has been modified to encode a modified rodent Acvr1 polypeptide, wherein the modified Acvr1 rodent polypeptide comprises the ectodomain of a human ACVR1 protein, and the transmembrane and cytoplasmic domains of a rodent Acvr1 protein except wherein Serine at position 330 in the cytoplasmic domain of the rodent Acvr1 protein has been substituted for Pro (a S330P substitution). In some embodiments, a modified rodent Acvr1 polypeptide comprises the signal peptide of a rodent Acvr1 protein. In some embodiments, the 5″ and 3′ untranslated regions (UTRs) of a rodent Acvr1 gene remain unmodified.

In some embodiments, in addition to encoding the ectodomain of a human ACVR1 protein and a S330P substitution, a modified rodent Acvr1 gene further comprises an FOP mutation. In some embodiments, an FOP mutation results from substitution of one or more nucleotides in the codon for Arg 206 of a rodent Acvr1 gene to code for His instead-such FOP mutation is also referred to as encoding a R206H substitution, or simply as a R206H mutation. In some embodiments, an FOP mutation results from substitution of one or more nucleotides in the codon for Arg 258 of a rodent Acvr1 gene to code for Gly instead-such FOP mutation is also referred to as encoding a R258G substitution, or simply as a R258G mutation.

In some embodiments, a modified Acvr1 gene that comprises an FOP mutation is derived from an engineered Acvr1 gene with a FlEx design. Although an engineered Acvr1 gene with a FlEx design is itself a modified Acvr1 gene, the term “engineered Acvr1 gene” is used herein to refer to Acvr1 genes with a FIEx design, to differentiate from modified Acvr1 genes without a FIEx design and from modified Acvr1 genes derived from an engineered Acvr1 gene with a FlEx design. A FIEx design provides for a conditional deletion of a wild-type exon and replacement of the wild-type exon with a mutant exon (e.g., an exon comprising an FOP mutation). FlEx allows for forming a conditional allele by placement of a mutant exon in the antisense orientation (hereon referred to as “inverted mutant exon”) next to a wild-type exon in the sense orientation that can be deleted. By utilizing selected site-specific recombinase recognition sites (SRRS's), in presence of their cognate recombinase, the inverted mutant exon is brought to the sense strand, and hence also in frame with the rest of the gene, whereas the wild-type exon is deleted. This FlEx approach relies on the placement of incompatible SRSS's (e.g., lox2372 and loxP) surrounding the wild-type and mutant exons. One advantage of the FIEx approach is that a lethal mutation (such as a perinatal/embryonic lethal mutation) is not expressed unless the FIEx allele is acted upon by the selected recombinase(s).

In some embodiments, a modified rodent Acvr1 gene, which comprises an FOP mutation and one or more modifications described herein (e.g., a modification to encode the ectodomain of a human ACVR1 protein and/or a modification to encode a S330P substitution), is derived from an engineered Acvr1 gene with a FlEx design.

In some embodiments, an engineered Acvr1 gene comprises (i) a nucleotide sequence encoding the ectodomain of a human ACVR1 protein, (ii) a nucleotide sequence comprising a codon encoding Pro at position 330; and (iii) a mutant rodent Acvr1 exon comprising an FOP mutation in antisense orientation flanked by a first pair of SRRS's, and a wild-type Acvr1 exon in sense orientation flanked by a second pair of SRRS's; wherein the first and second pairs of SRRS's are oriented to direct inversion of the mutant rodent Acvr1 exon into sense orientation and deletion of the wild-type Acvr1 exon, thereby forming a modified rodent Acvr1 gene encoding a modified Acvr1 polypeptide, wherein the modified Acvr1 polypeptide comprises the ectodomain of a human ACVR1 protein, and the transmembrane and cytoplasmic domains of a rodent Acvr1 protein except for the Pro at position 330 and the FOP mutation.

In some embodiments of an engineered Acvr1 gene with a FlEx design, the mutant exon is a mutant rodent exon 4 encoding a R206H mutation. In some embodiments of an engineered Acvr1 gene with a FIEx design, the mutant exon is a mutant rodent exon 5 encoding a R258G mutation.

In some embodiments of an engineered Acvr1 gene with a FlEx design, the rodent is mouse, and the engineered Acvr1 gene comprises (i) a nucleotide sequence encoding the ectodomain of a human ACVR1 protein, (ii) a nucleotide sequence comprising a codon encoding Pro at amino acid position 330 (in lieu of Ser in a mouse Acvr1 protein); and (iii) a mutant mouse Acvr1 exon comprising an FOP mutation in antisense orientation flanked by a first pair of SRRS's, and a wild-type Acvr1 exon in sense orientation flanked by a second pair of SRRS's, and wherein the first and second pairs of SRRS's are oriented to direct inversion of the mutant mouse Acvr1 exon into sense orientation and deletion of the wild-type Acvr1 exon, thereby forming a modified mouse Acvr1 gene encoding a modified Acvr1 polypeptide, wherein the modified Acvr1 polypeptide comprises the ectodomain of a human ACVR1 protein, and the transmembrane and cytoplasmic domains of a mouse Acvr1 protein except for the Pro at position 330 and the FOP mutation.

In some embodiments, an engineered Acvr1 gene with a FlEx design comprises a mouse Acvr1 exon 1, a modified mouse Acvr1 exon 2 which encodes a Q30P substitution, a mouse Acvr1 exon 3, a mutant mouse Acvr1 exon 4 comprising an R206H mutation in antisense orientation flanked by a first pair of SRRS's, a wild-type Acvr1 exon 4 in sense orientation flanked by a second pair of SRRS's, a mouse Acvr1 exon 5, a modified mouse Acvr1 exon 6 which encodes a S330P substitution, and mouse Acvr1 exons 7-9; wherein the first and second pairs of SRRS's are oriented to direct inversion of the mutant mouse Acvr1 exon 4 into sense orientation and deletion of the wild-type Acvr1 exon 4, thereby forming a modified mouse Acvr1 gene encoding a modified Acvr1 polypeptide, wherein the modified Acvr1 polypeptide comprises the ectodomain of a human ACVR1 protein, and the transmembrane and cytoplasmic domains of a mouse Acvr1 protein except for the R206H mutation and the S330P substitution.

In some embodiments, an engineered mouse Acvr1 gene with a FlEx design comprises a mouse Acvr1 exon 1, a modified mouse Acvr1 exon 2 which encodes a Q30P substitution, a mouse Acvr1 exon 3, a mouse Acvr1 exon 4, a mutant mouse Acvr1 exon 5 encoding an R258G mutation in antisense orientation flanked by a first pair of SRRS's, a wild-type Acvr1 exon 5 in sense orientation flanked by a second pair of SRRS's, a modified mouse Acvr1 exon 6 which encodes a S330P substitution, and mouse Acvr1 exons 7-9; wherein the first and second pairs of SRRS's are oriented to direct inversion of the mutant mouse Acvr1 exon 5 into sense orientation and deletion of the wild-type Acvr1 exon 5, thereby forming a modified mouse Acvr1 gene encoding a modified Acvr1 polypeptide, wherein the modified Acvr1 polypeptide comprises the ectodomain of a human ACVR1 protein, and the transmembrane and cytoplasmic domains of a mouse Acvr1 protein except for the R258G mutation and the S330P substitution.

In some embodiments of an engineered rodent Acvr1 gene with a FIEx design, the wild-type exon that is in sense orientation to be subsequently deleted is an exon (i.e., wild type exon) of a human ACVR1 gene. In some embodiments of an engineered rodent Acvr1 gene with a FlEx design, the wild-type exon that is in sense orientation to be subsequently deleted is an exon encoding the same amino acids as a human ACVR1 exon but having a reduced nucleotide sequence identity with the mutant rodent Acvr1 exon to be inverted as compared to the human ACVR1 exon. A reduced sequence identity with the mutant rodent Acvr1 exon may reduce undesirable recombination or rearrangement events.

In some embodiments of an engineered rodent Acvr1 gene with a FIEx design, the first pair of SRRS' includes a first SRRS and a second SRRS, wherein the first and second SRRS' are compatible with each other and are oriented to direct an inversion. In some embodiments, the second pair of SRRS' includes a third SRRS and a fourth SRRS, wherein the third and fourth SRRS' are compatible with each other, are oriented to direct an inversion, but are not compatible with the first or second SRRS. In some embodiments, all SRRS' are recognized by the same recombinase, such as Cre. In some embodiments, the first pair of SRRS' is a pair of Lox2372 sites, and the second pair of SRRS' is a pair of LoxP sites. In some embodiments, the first pair of SRRS' is a pair of LoxP sites, and the second pair of SRRS' is a pair of Lox2372 sites.

### Genetically Modified Rodents, Rodent Tissues and Cells

Genetically modified rodents are provided that express a modified Acvr1 protein comprising an FOP mutation that results in a disorder characterized by ectopic bone formation.

In some embodiments, disclosed herein is a genetically modified rodent comprising a modified rodent Acvr1 gene as described above, wherein the modified rodent Acvr1 gene is at an endogenous rodent Acvr1 locus and under control of the endogenous rodent Acvr1 promoter.

In some embodiments, a modified rodent (e.g., mouse or rat) Acvr1 gene results from a modification to an endogenous rodent Acvr1 gene at an endogenous rodent Acvr1 locus.

In some embodiments, a modification to an endogenous rodent Acvr1 gene comprises substitution of one or more nucleotides in the coding sequence for the ectodomain of the rodent Acvr1 protein to code for the ectodomain of a human ACVR1 protein. In some embodiments, a modification to an endogenous rodent Acvr1 gene comprises replacement of the coding sequence for the entire ectodomain of an endogenous rodent Acvr1 protein with a coding sequence for the entire ectodomain of a human ACVR1 protein. Because of the high degree of sequence identity across species, it is not always necessary to replace the coding sequence for the entire ectodomain of an endogenous rodent Acvr1 protein in order for the modified Acvr1 gene to code for the ectodomain of a human ACVR1 protein. For example, the ectodomains of human and mouse Acvr1 proteins differ only at amino acid at position 30, with Gln (Q) for mouse Acvr1 protein and Pro (P) for human ACVR1 protein. Thus, modification to an endogenous mouse Acvr1 gene to substitute one or more nucleotides in codon 30 to code for Pro instead would result in a modified mouse Acvr1 gene encoding a modified mouse Acvr1 protein having the ectodomain of a human ACVR1 protein.

In some embodiments, a modification to an endogenous rodent Acvr1 gene comprises substitution of one or more nucleotides in the coding sequence for the ectodomain of an endogenous rodent Acvr1 protein such that the resulting modified rodent Acvr1 gene encodes the entire ectodomain of a human ACVR1 protein. In some embodiments, a modification is made to an endogenous mouse Acvr1 gene, which modification comprises substitution of one or more nucleotides in the codon for amino acid Glutamine at position 30 (Q30) to code for Proline instead, resulting in a modified mouse Acvr1 gene which encodes the entire ectodomain of a human ACVR1 protein.

In some embodiments, a modification to an endogenous rodent Acvr1 gene comprises replacement of a contiguous sequence coding for amino acids within the ectodomain of an endogenous rodent Acvr1 protein such that the resulting modified rodent Acvr1 gene encodes the entire ectodomain of a human ACVR1 protein. In some embodiments, a modification is made to an endogenous mouse Acvr1 gene, which modification comprises a replacement of a contiguous nucleic acid sequence in exon 2 of the endogenous mouse Acvr1 gene coding for amino acids surrounding and including Q30, with a contiguous nucleic acid sequence in exon 2 of a human ACVR1 gene coding for the corresponding amino acids of the human ACVR1 protein. In some embodiments, the contiguous nucleic acid sequence in exon 2 of an endogenous mouse Acvr1 gene that is being replaced encodes about 5-45 amino acids including Q30, or about 10-40 amino acids including Q30, or about 20-35 amino acids including Q30, or about 25-35 amino acids including Q30. In some embodiments, the contiguous nucleic acid sequence in exon 2 that is being replaced encodes the amino acid sequence of EKPKVNQKLYMCVCEGLSCGNEDHCE (SEQ ID NO: 40) (Q in this sequence representing Q30).

In some embodiments, a modification to an endogenous rodent Acvr1 gene comprises substitution of one or more nucleotides in the codon for amino acid Serine at position 330 (S330) to code for Proline instead, which is the amino acid found at position 330 of a human ACVR1 protein. Amino acid 330 is in the cytoplasmic domain, and the codon for amino acid 330 is in exon 6 for both human and rodent (e.g., mouse) Acvr1 genes. In some embodiments, a modification to an endogenous rodent Acvr1 gene comprises a replacement of a contiguous nucleic acid sequence in exon 6 of the endogenous rodent Acvr1 gene coding for amino acids surround and including S330 of a rodent Acvr1 protein, with a contiguous nucleic acid sequence in exon 6 of a human ACVR1 gene coding for the corresponding amino acids (including P330) of the human ACVR1 protein. In some embodiments, the contiguous nucleic acid sequence in exon 6 of an endogenous rodent Acvr1 gene that is being replaced encodes about 5-45 amino acids including S330.

In some embodiments, an endogenous rodent Acvr1 gene has been modified to comprise a combination of modifications described above. In some embodiments, an endogenous rodent Acvr1 gene has been modified to encode a modified Acvr1 polypeptide, wherein the modified Acvr1 polypeptide comprises the ectodomain of a human ACVR1 protein, and the transmembrane and cytoplasmic domains of an endogenous rodent Acvr1 protein except for a S330P substitution.

In some embodiments, a modified rodent Acvr1 polypeptide comprises the signal peptide of a rodent Acvr1 protein (e.g., an endogenous rodent Acvr1 protein). In some embodiments, the 5 and 3′ untranslated regions (UTRs) of an endogenous rodent Acvr1 gene remain unmodified.

In some embodiments, in addition to encoding the ectodomain of a human ACVR1 protein and a S330P substitution, a modified rodent Acvr1 gene further comprises an FOP mutation. In some embodiments, an FOP mutation results from substitution of one or more nucleotides in the codon for Arg 206 of a rodent Acvr1 gene to code for His instead-such FOP mutation is also referred to as encoding a R206H substitution, or simply as a R206H mutation. In some embodiments, an FOP mutation results from substitution of one or more nucleotides in the codon for Arg 258 of a rodent Acvr1 gene to code for Gly instead-such FOP mutation is also referred to as encoding a R258G substitution, or simply as a R258G mutation.

In some embodiments, a genetically modified rodent comprises a modified rodent Acvr1 gene in its genome (i.e., germline genome). In some embodiments, a genetically modified rodent comprises a modified rodent Acvr1 gene in its genome, wherein the modified rodent Acvr1 gene encodes a modified rodent Acvr1 polypeptide comprising the ectodomain of a human ACVR1 protein, and the transmembrane and cytoplasmic domains of an endogenous rodent Acvr1 protein except for a S330P substitution. In some embodiments, a genetically modified rodent comprises a modified rodent Acvr1 gene in its genome, wherein the modified rodent Acvr1 gene encodes a modified rodent Acvr1 polypeptide comprising the ectodomain of a human ACVR1 protein, the transmembrane and cytoplasmic domains of an endogenous rodent Acvr1 protein except for a S330P substitution and an FOP mutation (such as a R206H substitution or a R258G substitution).

In some embodiments, a genetically modified rodent comprises a modified rodent Acvr1 gene comprising an FOP mutation, wherein the modified rodent Acvr1 gene, instead of being in the genome of the rodent, is derived at an embryonic stage of the rodent from an engineered Acvr1 gene comprising an FOP mutation with a FIEx design in the rodent genome.

In some embodiments, a genetically modified rodent comprises a modified rodent Acvr1 gene derived at an embryonic stage of the rodent from an engineered Acvr1 gene which comprises (i) a nucleotide sequence encoding the ectodomain of a human ACVR1 protein, (ii) a nucleotide sequence comprising a codon encoding Pro at position 330; and (iii) a mutant rodent Acvr1 exon comprising an FOP mutation in antisense orientation flanked by a first pair of SRRS's, and a wild-type Acvr1 exon in sense orientation flanked by a second pair of SRRS's; wherein the first and second pairs of SRRS's are oriented to direct inversion of the mutant rodent Acvr1 exon into sense orientation and deletion of the wild-type Acvr1 exon, thereby forming a modified rodent Acvr1 gene encoding a modified rodent Acvr1 polypeptide, wherein the modified rodent Acvr1 polypeptide comprises the ectodomain of a human ACVR1 protein, and the transmembrane and cytoplasmic domains of an endogenous rodent Acvr1 protein except for the Pro at position 330 and the FOP mutation. In some embodiments, the mutant exon is a mutant rodent exon 4 encoding a R206H mutation. In some embodiments, the mutant exon is a mutant rodent exon 5 encoding a R258G mutation.

In some embodiments, the rodent is a mouse which comprises a modified mouse Acvr1 gene derived at an embryonic stage from an engineered Acvr1 gene comprising (i) a nucleotide sequence encoding the ectodomain of a human ACVR1 protein, (ii) a nucleotide sequence comprising a codon encoding Pro at amino acid position 330 (in lieu of Ser in a mouse Acvr1 protein); and (iii) a mutant mouse Acvr1 exon comprising an FOP mutation in antisense orientation flanked by a first pair of SRRS's, and a wild-type Acvr1 exon in sense orientation flanked by a second pair of SRRS's, and wherein the first and second pairs of SRRS's are oriented to direct inversion of the mutant mouse Acvr1 exon into sense orientation and deletion of the wild-type Acvr1 exon, thereby forming a modified mouse Acvr1 gene encoding a modified mouse Acvr1 polypeptide, wherein the modified Acvr1 polypeptide comprises the ectodomain of a human ACVR1 protein, and the transmembrane and cytoplasmic domains of an endogenous mouse Acvr1 protein except for the S330P substitution and the FOP mutation.

In some embodiments, an engineered Acvr1 gene with a FlEx design comprises a mouse Acvr1 exon 1, a modified mouse Acvr1 exon 2 which encodes a Q30P substitution, a mouse Acvr1 exon 3, a mutant mouse Acvr1 exon 4 which encodes an R206H mutation in antisense orientation flanked by a first pair of SRRS's, a wild-type Acvr1 exon 4 in sense orientation flanked by a second pair of SRRS's, a mouse Acvr1 exon 5, a modified mouse Acvr1 exon 6 which encodes a S330P substitution, and mouse Acvr1 exons 7-9; wherein the first and second pairs of SRRS's are oriented to direct inversion of the mutant mouse Acvr1 exon 4 into sense orientation and deletion of the wild-type Acvr1 exon 4, thereby forming a modified mouse Acvr1 gene encoding a modified mouse Acvr1 polypeptide, wherein the modified mouse Acvr1 polypeptide comprises the ectodomain of a human ACVR1 protein, and the transmembrane and cytoplasmic domains of an endogenous mouse Acvr1 protein except for the R206H mutation and the S330P substitution.

In some embodiments, an engineered Acvr1 gene with a FlEx design comprises a mouse Acvr1 exon 1, a modified mouse Acvr1 exon 2 which encodes a Q30P substitution, mouse Acvr1 exons 3-4, a mutant mouse Acvr1 exon 5 encoding an R258G mutation in antisense orientation flanked by a first pair of SRRS's, a wild-type Acvr1 exon 5 in sense orientation flanked by a second pair of SRRS's, a modified mouse Acvr1 exon 6 which encodes a S330P substitution, and mouse Acvr1 exons 7-9; wherein the first and second pairs of SRRS's are oriented to direct inversion of the mutant mouse Acvr1 exon 5 into sense orientation and deletion of the wild-type Acvr1 exon 5, thereby forming a modified mouse Acvr1 gene encoding a modified mouse Acvr1 polypeptide, wherein the modified mouse Acvr1 polypeptide comprises the ectodomain of a human ACVR1 protein, and the transmembrane and cytoplasmic domains of an endogenous mouse Acvr1 protein except for the R258G mutation and the S330P substitution.

In some embodiments, the wild-type Acvr1 exon that is in sense orientation to be subsequently deleted is an exon of a human ACVR1 gene. In some embodiments, the wild-type exon that is in sense orientation to be subsequently deleted is an exon encoding the same amino acids as a human ACVR1 exon but having a reduced nucleotide sequence identity with the mutant rodent Acvr1 exon to be inverted as compared to the human ACVR1 exon.

In some embodiments, the first pair of SRRS' includes a first SRRS and a second SRRS, wherein the first and second SRRS' are compatible with each other and are oriented to direct an inversion. In some embodiments, the second pair of SRRS' includes a third SRRS and a fourth SRRS, wherein the third and fourth SRRS' are compatible with each other, are oriented to direct an inversion, but are not compatible with the first or second SRRS. In some embodiments, all SRRS' are recognized by the same recombinase, such as Cre. In some embodiments, the first pair of SRRS' is a pair of Lox2372 sites, and the second pair of SRRS' is a pair of LoxP sites. In some embodiments, the first pair of SRRS' is a pair of LoxP sites, and the second pair of SRRS' is a pair of Lox2372 sites.

In some embodiments, a genetically modified rodent comprising an engineered rodent Acvr1 gene having an FOP mutation in a FIEx design expresses a recombinase at an embryonic stage in the rodent to direct an inversion of the mutant Acvr1 exon comprising the FOP mutation into sense orientation and deletion of the wild type Acvr1 exon, thereby forming a modified rodent Acvr1 gene at an embryonic stage in the rodent. In some embodiments, expression of the recombinase at an embryonic stage of the rodent is achieved by placing a coding sequence of the recombinase under control of a promoter active at an embryonic stage of the rodent. Suitable promoters include, for example, a Nanog promoter (see, e.g., Mitsui et al., Cell 113:631-642 (2003); Chambers, et al., Cell 113:643-655 (2003); both incorporated herein by reference), a Sox2 promoter, and a CMV promoter. The coding sequence of a recombinase, operably linked to a promoter active at an embryonic stage, can be integrated in the genome of a rodent.

In various embodiments, the rodent is selected from the group consisting of a mouse, a rat, and a hamster. In some embodiments, the rodent is a mouse. In some embodiments, the rodent is a rat.

In some embodiments, a rodent is heterozygous for a modified rodent Acvr1 gene. In some embodiments, a rodent is homozygous for a modified rodent Acvr1 gene.

In some embodiments, disclosed herein are isolated rodent tissue or cells comprising a modified rodent Acvr1 gene described herein. In some embodiments, such tissue or cell can be isolated from a genetically modified rodent described herein that comprises a modified rodent Acvr1 gene. In some embodiments, the rodent cell is a sperm cell or an egg. In some embodiments, a rodent cell comprising a modified rodent Acvr1 gene is a rodent embryonic stem (ES) cell. In some embodiments, a rodent ES cell is a mouse ES cell; and in some embodiments, a rodent ES cell is a rat ES cell.

### Vectors And Methods For Making Genetically Modified Rodents and Rodent ES Cells

A targeting nucleic acid construct comprising a modified rodent Acvr1 gene described above, or a portion thereof comprising desired modification(s), is disclosed herein for introducing the modified Acvr1 gene or a portion thereof into a rodent genome. In addition to a modified rodent Acvr1 gene or a portion thereof comprising desired modification(s), the nucleic acid construct can include flanking sequences that are of suitable lengths and substantial identity to rodent sequences at an endogenous rodent Acvr1 locus so as to be capable of mediating homologous recombination and integration of the modified rodent Acvr1 gene or a portion thereof into the endogenous rodent Acvr1 locus to form a modified rodent Acvr1 gene at the endogenous rodent Acvr1 locus. The substantial identity between a homology arm to endogenous rodent sequences is at least 90%, 95%, 98%, or greater. In some embodiments, a homology arm includes a nucleotide sequence identical to an endogenous rodent sequence at the endogenous rodent Acvr1 locus.

A targeting nucleic acid construct comprising an engineered rodent Acvr1 gene with a FlEx design described above, or a portion thereof comprising desired modification(s), is disclosed herein for introducing the engineered Acvr1 gene or a portion thereof into a rodent genome. In addition to an engineered rodent Acvr1 gene or a portion thereof comprising desired modification(s), the nucleic acid construct can include flanking sequences that are of suitable lengths and substantial sequence identity to rodent sequences at an endogenous rodent Acvr1 locus so as to be capable of mediating homologous recombination and integration of the engineered Acvr1 gene with a FlEx design or a portion thereof into the endogenous rodent Acvr1 locus, to form the engineered Acvr1 gene with a FlEx design at the endogenous rodent Acvr1 locus. The substantial identity between a homology arm to endogenous rodent sequences is at least 90%, 95%, 98%, or greater. In some embodiments, a homology arm includes a nucleotide sequence identical to an endogenous rodent sequence at the endogenous rodent Acvr1 locus.

In some embodiments, a targeting nucleic acid construct is introduced into a rodent embryonic stem (ES) cell to modify the genome of the ES cell. Both mouse ES cells and rat ES cells have been described in the art. See, e.g., U.S. Pat. Nos. 7,576,259, 7,659,442, and 7,294,754, and US Publ. No. 2008/0078000 A1 (all of which are incorporated herein by reference) describe mouse ES cells and the VELOCIMOUSE® method for making a genetically modified mouse; and US Publ. No. 2014/0235933 A1 and US Publ. No. 2014/0310828 A1 (all of which are incorporated herein by reference) describe rat ES cells and methods for making a genetically modified rat.

ES cells having a modified or engineered Acvr1 gene at the endogenous rodent Acvr1 locus can be identified and selected. The selected positively targeted ES cells are then used as donor ES cells for injection into a pre-morula stage embryo (e.g., 8-cell stage embryo) by using the VELOCIMOUSE® method (see, e.g., U.S. Pat. Nos. 7,576,259, 7,659,442, and 7,294,754, and US Publ. No. 2008/0078000 A1), or methods described in US Publ. Nos. 2014/0235933 A1 and 2014/0310828 A1. The embryo comprising the donor ES cells is incubated until blastocyst stage and then implanted into a surrogate mother to produce an F0 rodent fully derived from the donor ES cells. Rodent pups bearing the modified or engineered Acvr1 gene can be identified by genotyping of DNA isolated from tail snips using, for example, a loss of allele assay (Valenzuela et al., supra).

In some embodiments, a genetically modified rodent comprising an engineered Acvr1 gene with a FlEx design, also referred to as an engineered Acvr1 FlEx allele, is made by modifying a rodent ES cell to contain the engineered Acvr1 FlEx allele, and modifying the same ES cell to contain a gene encoding a recombinase (e.g., Cre) operably linked to a promoter active in an embryonic stage, and using the ES cell as a donor cell to make a rodent that contains the engineered Acvr1 FlEx allele and the gene encoding the recombinase. In other embodiments, a genetically modified rodent comprising an engineered Acvr1 FlEx allele is made and crossed with a rodent containing a gene encoding a recombinase (e.g., Cre) operably linked to a promoter active in an embryonic stage, to obtain an offspring that contains the engineered Acvr1 FlEx allele and the gene encoding the recombinase which is active in an embryonic stage to convert the engineered Acvr1 FlEx allele into a modified Acvr1 gene which expresses a modified Acvr1 protein comprising an FOP mutation.

### Use of the Rodent as a Model of FOP

As described herein, by humanizing a rodent mutant Acvr1 protein comprising a FOP mutation (such as R206H mutation) through substituting the rodent Acvr1 ectodomain with the human ACVR1 ectodomain and substituting Serine 330 with a Proline, as is found in human ACVR1, neonatal lethality is alleviated and the rodent animal can survive at least 14-23 days. The resulting rodent exhibits phenotypes characteristics of FOP, e.g., congenital toe malformations and injury-induced and idiopathic HO in post-natal life. Hence, the genetically modified rodents described herein are suitable for use as a rodent model of FOP.

In some embodiments, a genetically modified rodent described herein may be used in the screening and development of therapeutic compounds for the inhibition, prevention, and/or treatment of ectopic bone disorders, including FOP.

In some embodiments, a candidate therapeutic compound is tested in vivo, by administering the compound to a genetically modified rodent disclosed herein.

Candidate therapeutic compounds can be, without limitation, small molecule chemical compounds, antibodies, inhibitory nucleic acids, or any combination thereof. In a specific embodiment, the compound is an antibody or antigen-binding fragment thereof, e.g., an activin A neutralizing antibody or antigen-binding fragment thereof, or an anti-Acvr1 antibody or antigen-binding fragment thereof. In some embodiments, the compound comprises an antagonist of one or more of activin receptor 1, activin receptor type 2A, and activin receptor type 2B. Any such antagonist may comprise an antibody. In some embodiments, the compound comprises an antibody against activin A. An antagonist or antibody against activin receptor 1, against activin receptor type 2A, against activin receptor type 2B, or against activin A may be any antagonist or antibody described or exemplified in U.S. Publ. No. 2018/0111983, which is incorporated by reference herein.

Administration of the compound can be performed before, during, or after induction of the recombinase activity in the rodent to allow the mutant Acvr1 allele to be expressed. Candidate therapeutic compounds may be dosed via any desired route of administration including parenteral and non-parenteral routes of administration. Parenteral routes include, e.g., intravenous, intraarterial, intraportal, intramuscular, subcutaneous, intraperitoneal, intraspinal, intrathecal, intracerebroventricular, intracranial, intrapleural or other routes of injection. Non-parenteral routes include, e.g., oral, nasal, transdermal, pulmonary, rectal, buccal, vaginal, ocular. Administration may also be by continuous infusion, local administration, sustained release from implants (gels, membranes or the like), and/or intravenous injection.

Various assays may be performed to determine the pharmacokinetic properties of administered compounds using samples obtained from rodent animals described. Pharmacokinetic properties include, but are not limited to, how a non-human animal processes the compound into various metabolites (or detection of the presence or absence of one or more metabolites, including, but not limited to, toxic metabolites), half-life, circulating levels (e.g., serum concentration), anti-compound response (e.g., antibodies), absorption and distribution, route of administration, routes of excretion and/or clearance of the compound.

In some embodiments, performing an assay includes determining the differences between a genetically modified rodent animal administered a compound and a genetically modified rodent animal not administered the compound, and determining whether the compound can inhibit the development and/or progression of ectopic bone formation in the rodent.

The present description is further illustrated by the following examples, which should not be construed as limiting in any way. The contents of all cited references (including literature references, issued patents, and published patent applications as cited throughout this application) are hereby expressly incorporated by reference.

### Example 1. Generation of Mice Having an Engineered Mouse Acvr1 Allele (“8431”): Comprising Q30P and S330P Humanization, Reversed FOP COIN Allele (R206H), and Containing Neo and Hygro Resistance Cassettes

The Mouse Acvr1 Locus was Modified by Using VELOCIGENE® Technology (See, e.g., U.S. Pat. No. 6,586,251 and Valenzuela et al. (2003) High-throughput engineering of the mouse genome coupled with high-resolution expression analysis, Nat. Biotech. 21 (6): 652-659, both incorporated herein by reference).

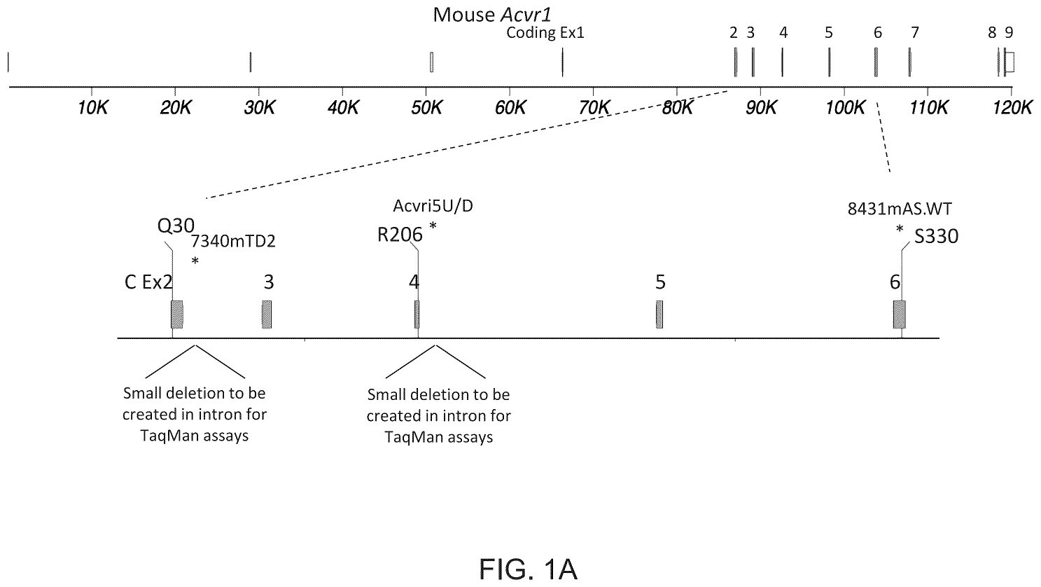

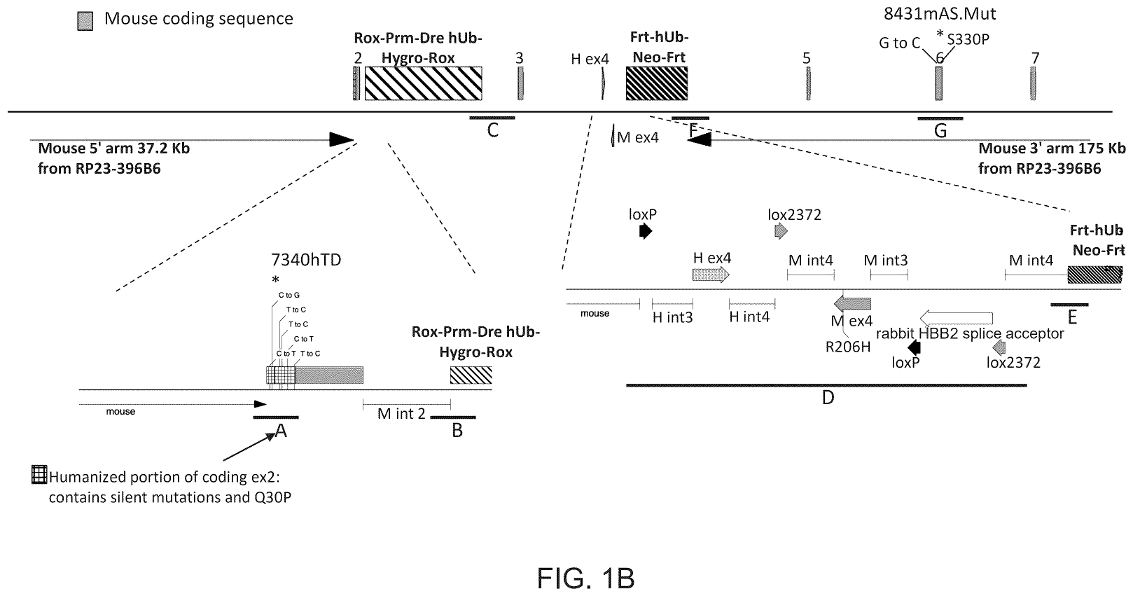

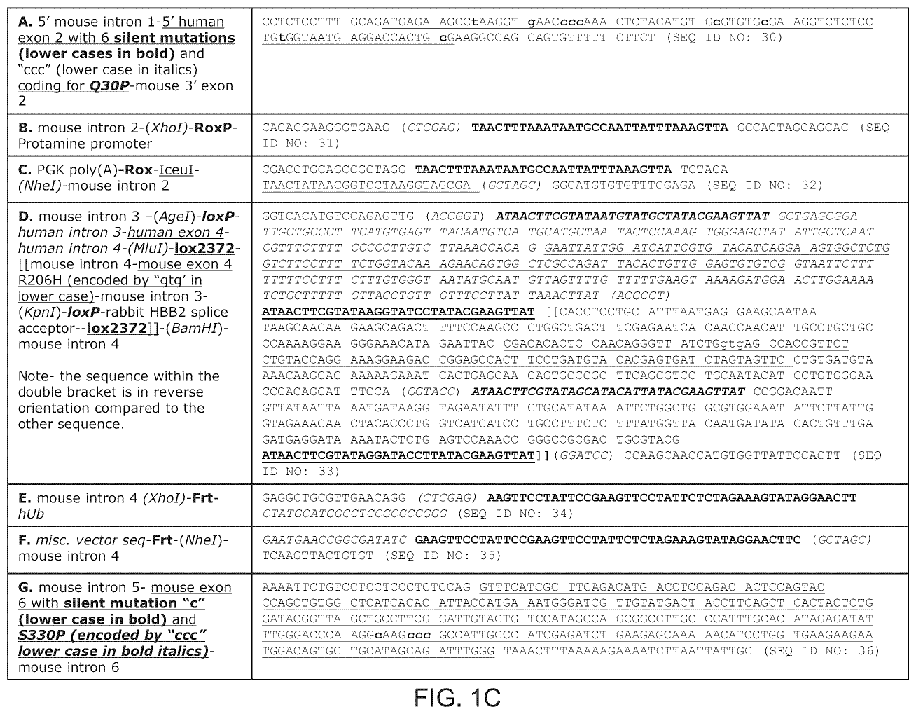

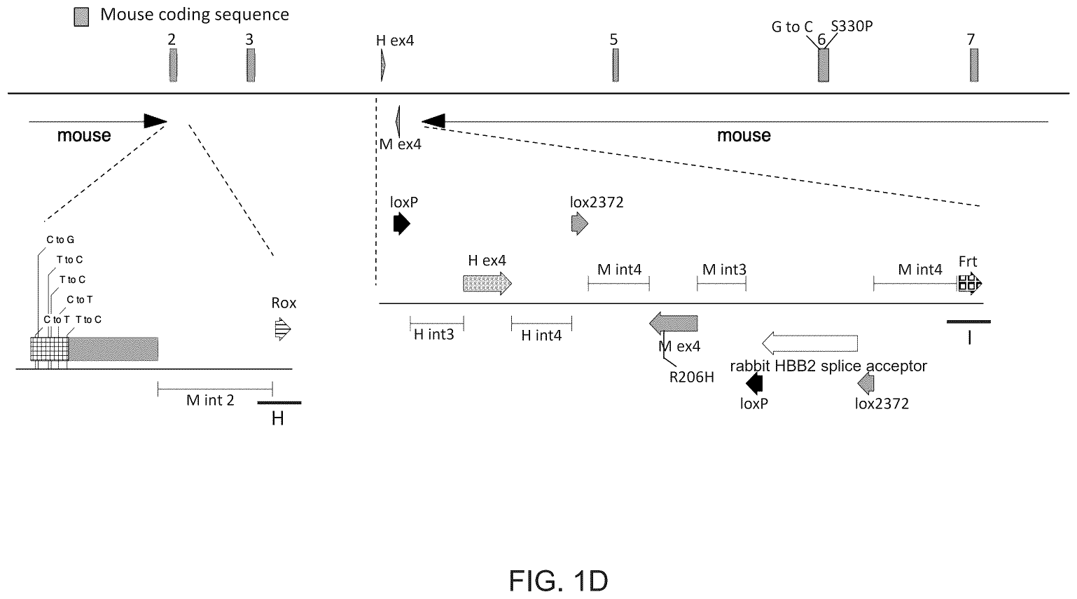

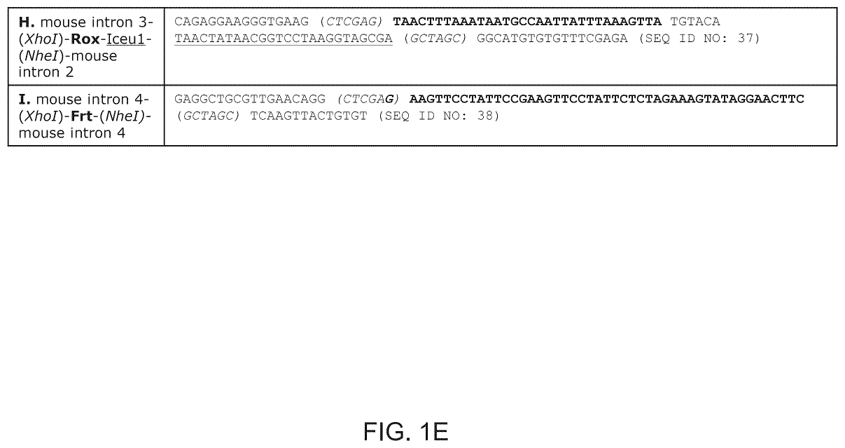

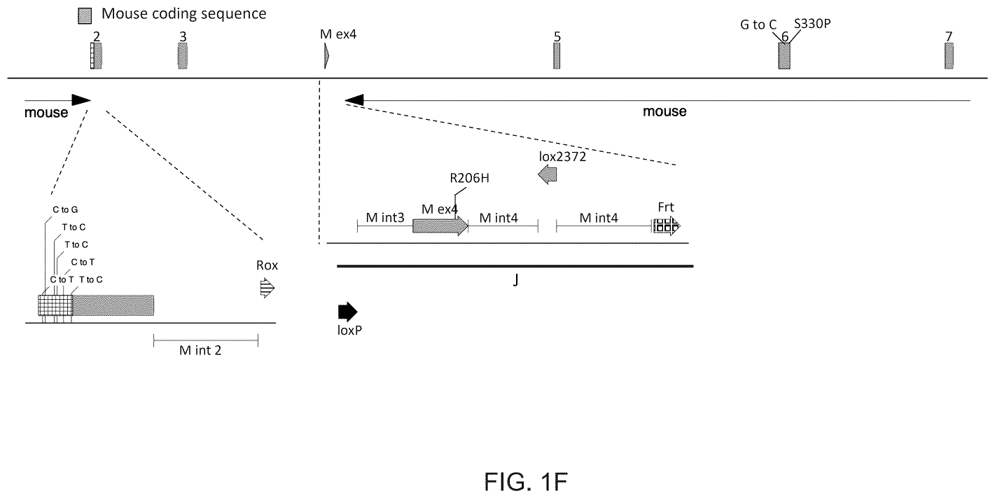

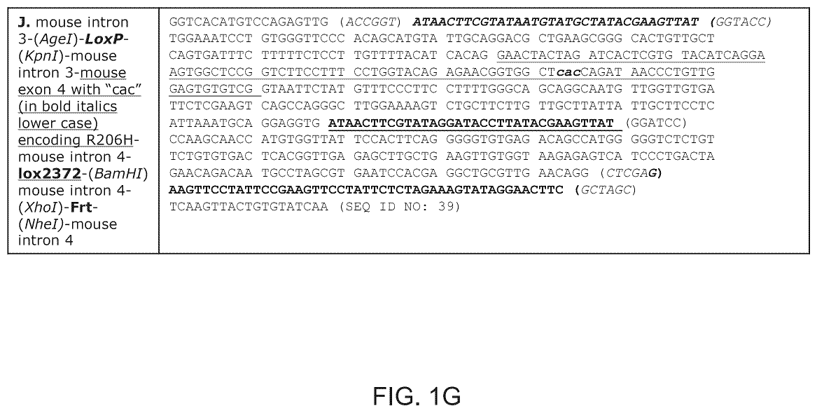

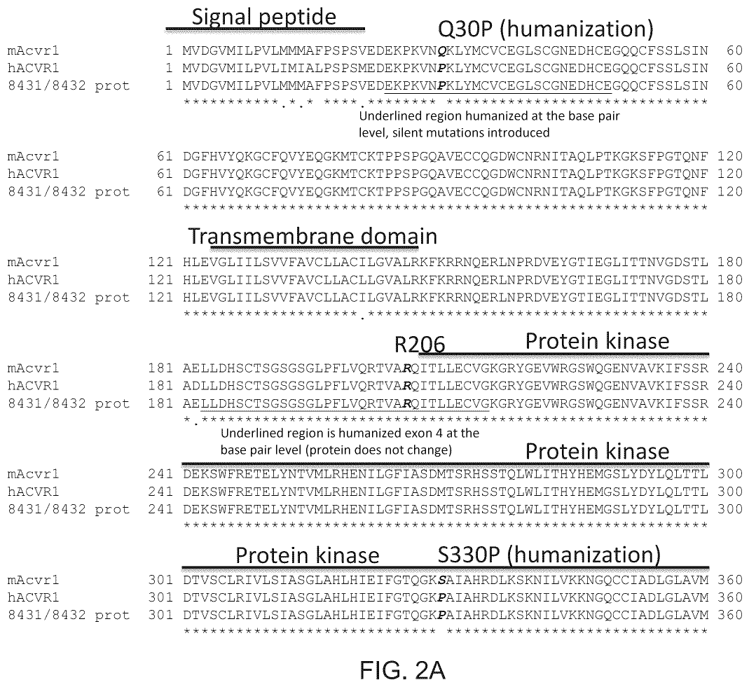

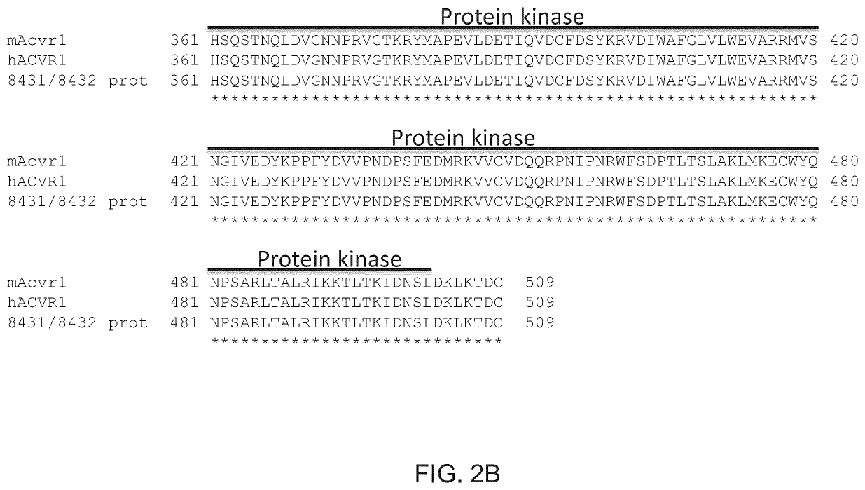

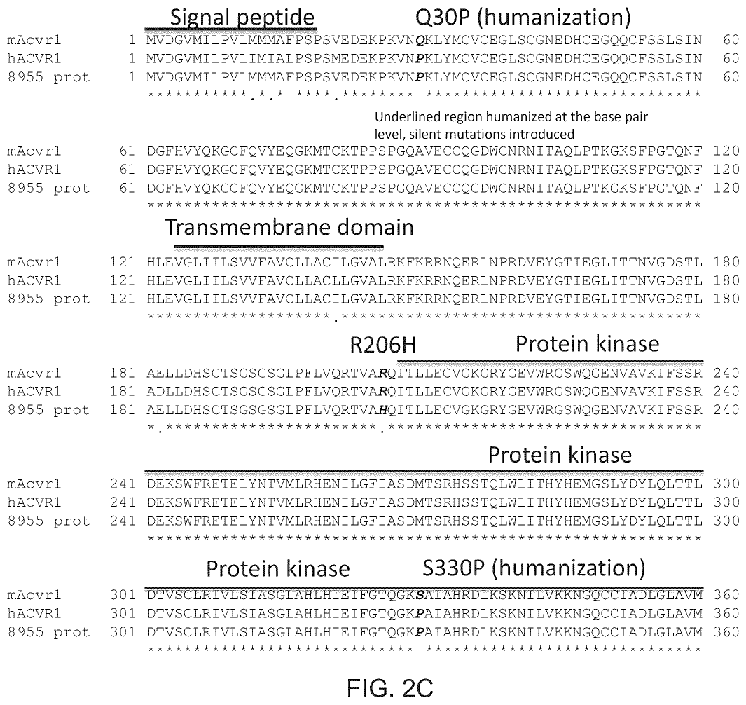

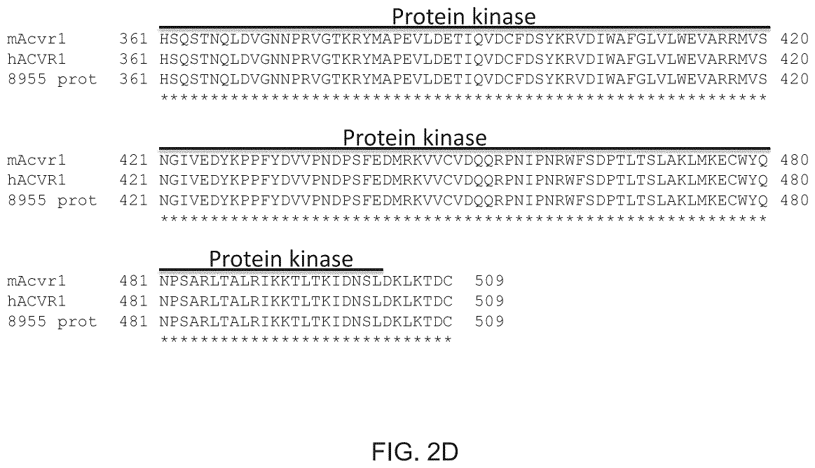

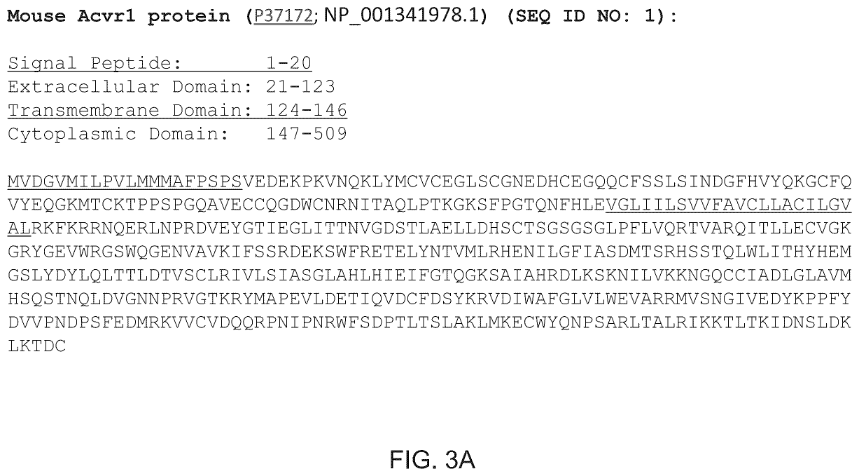

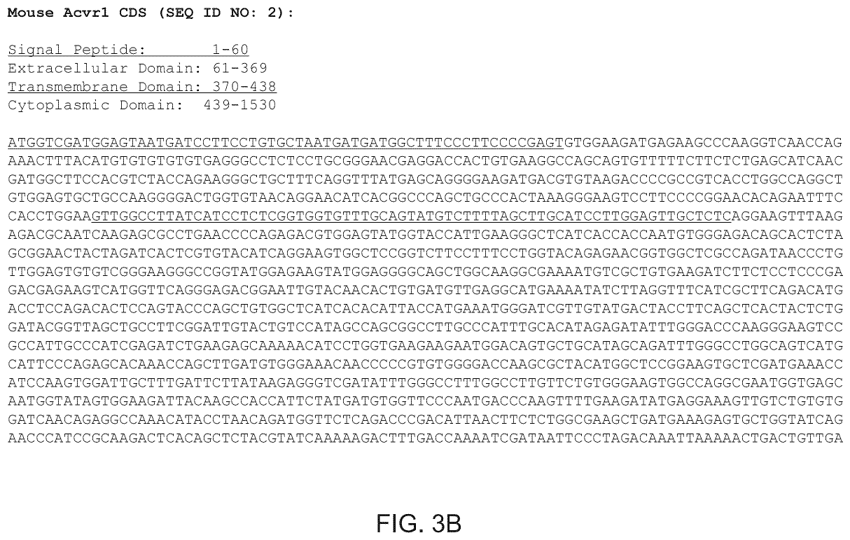

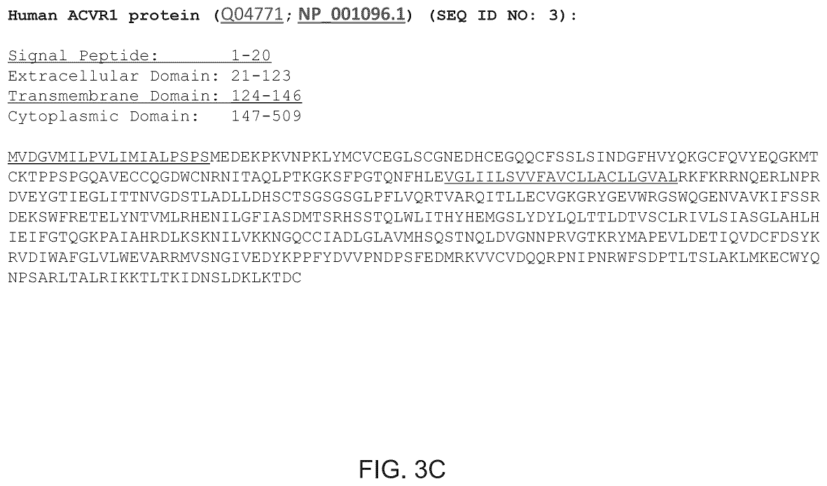

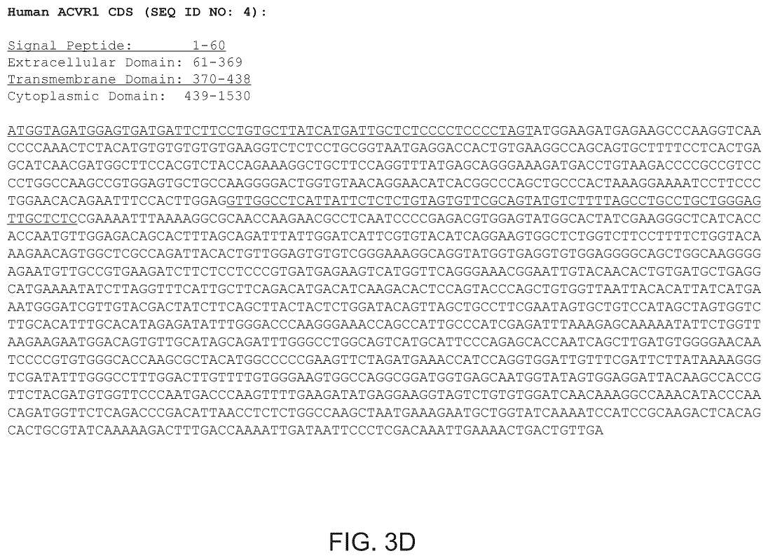

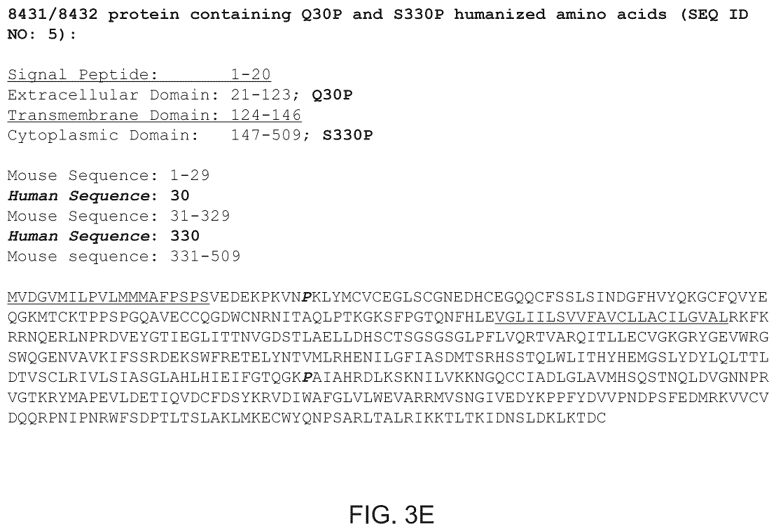

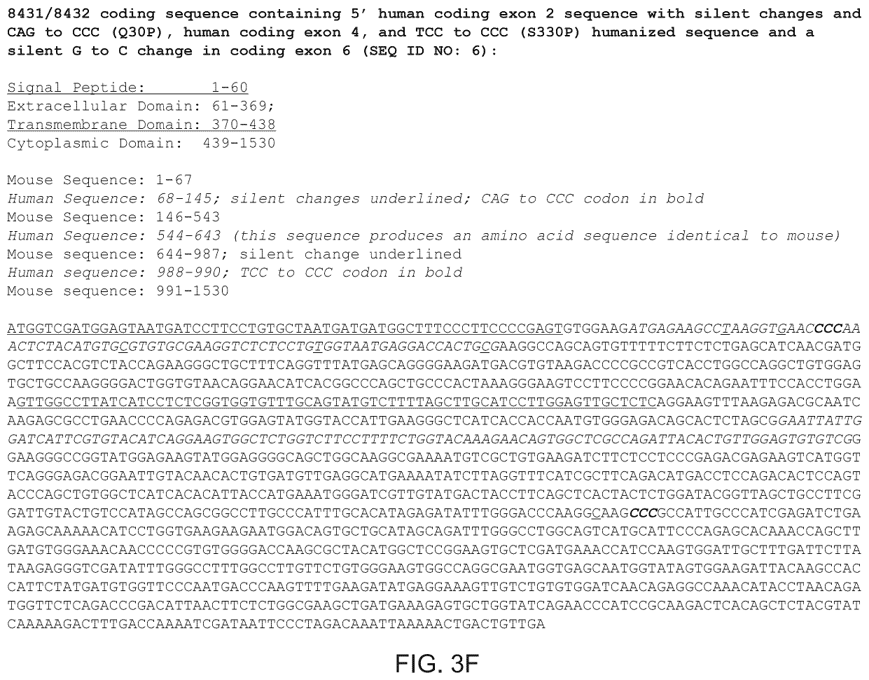

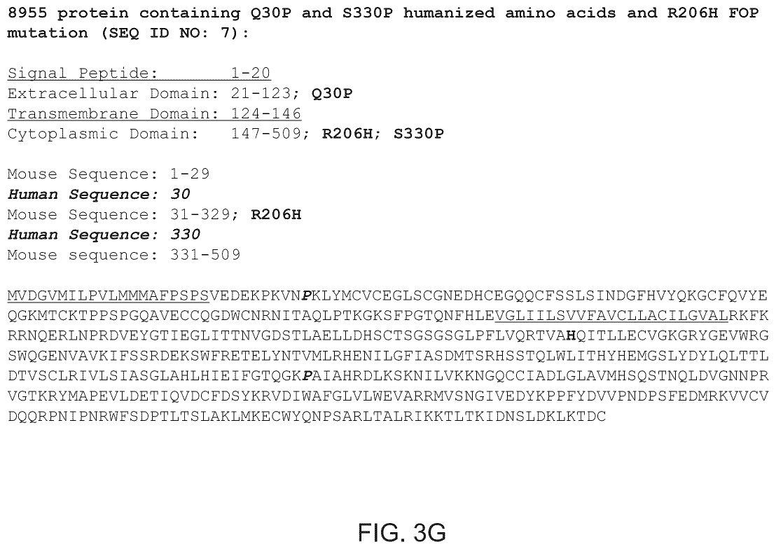

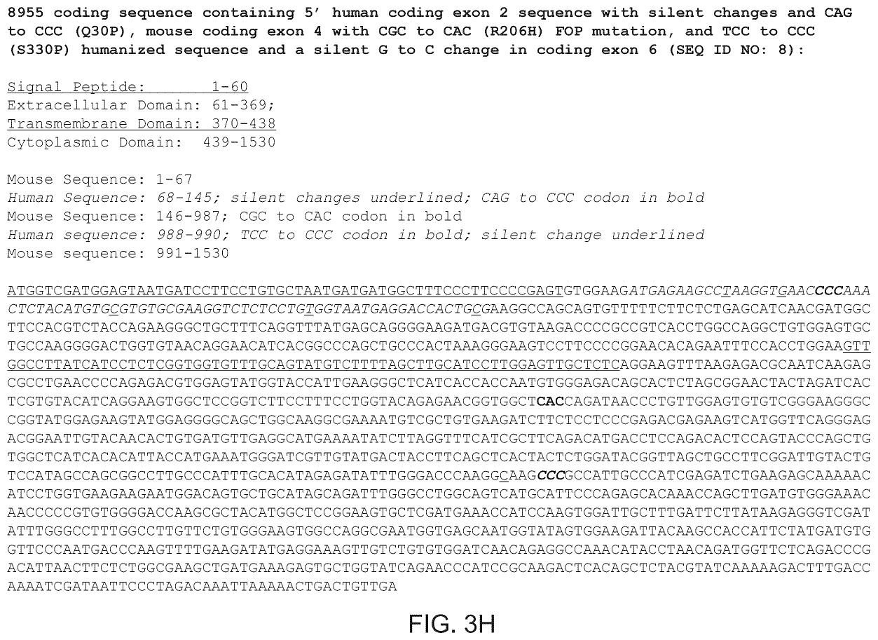

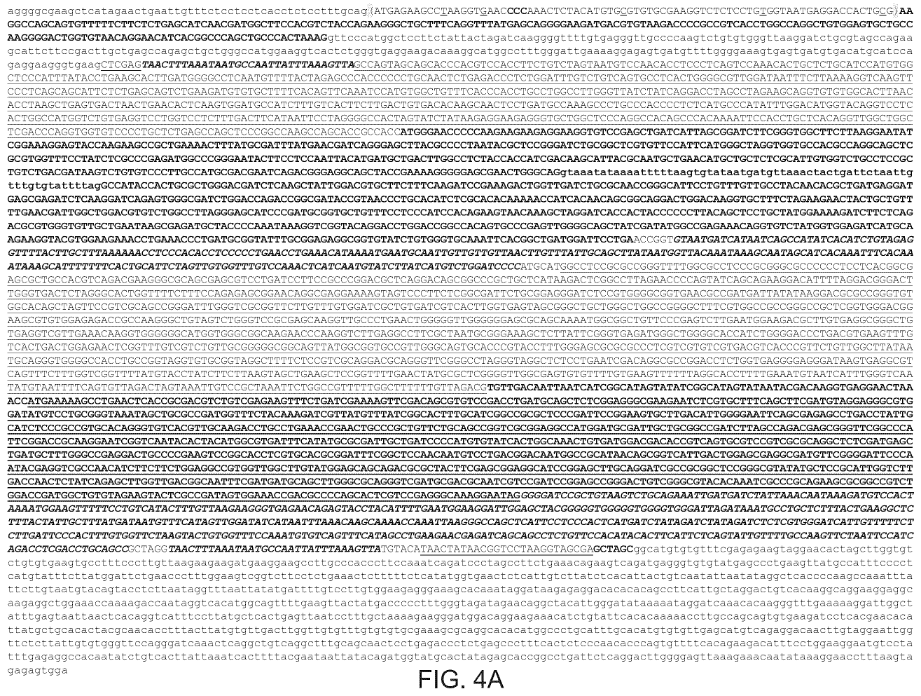

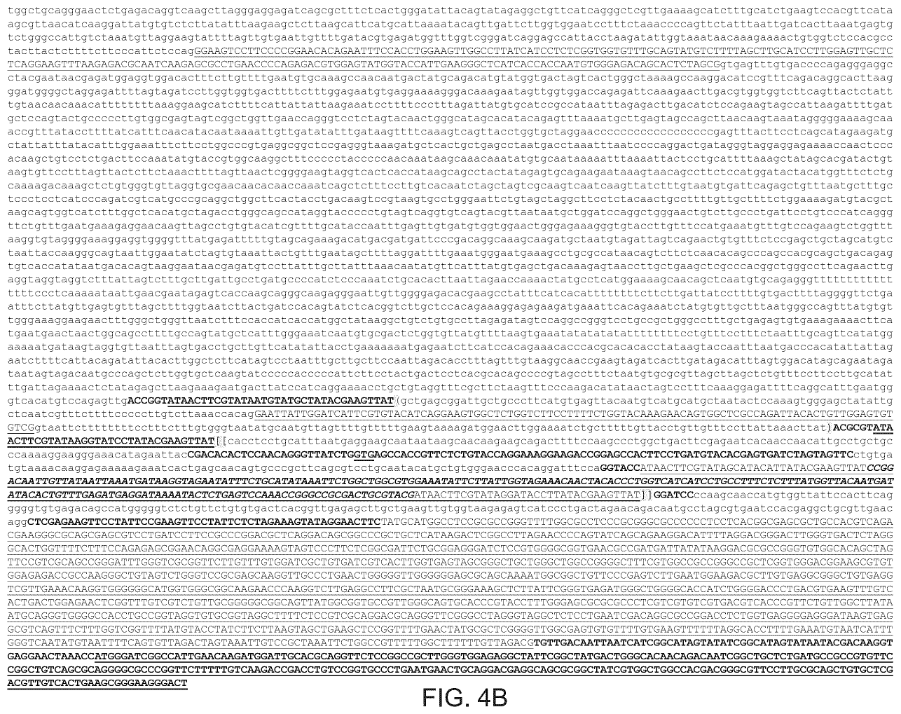

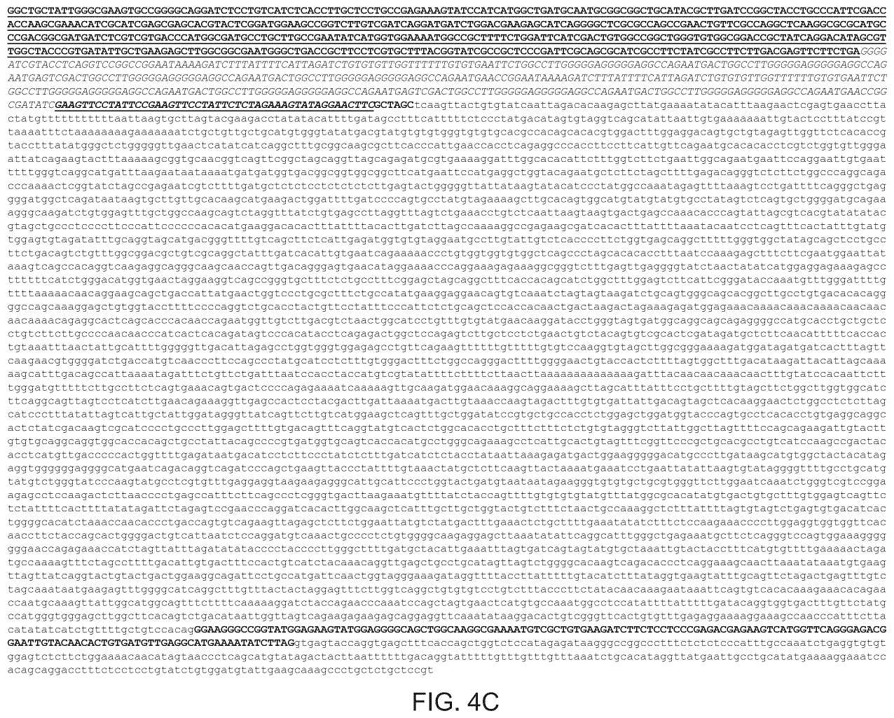

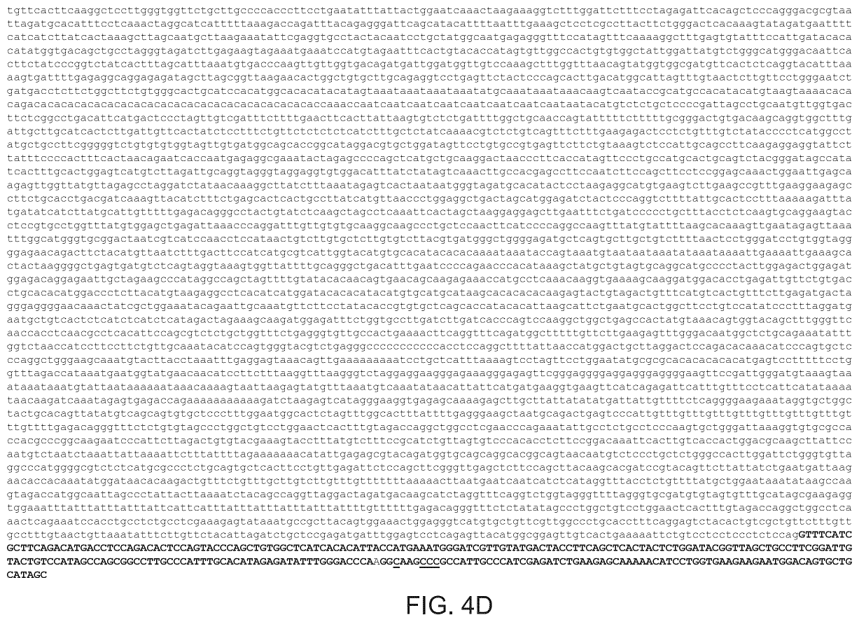

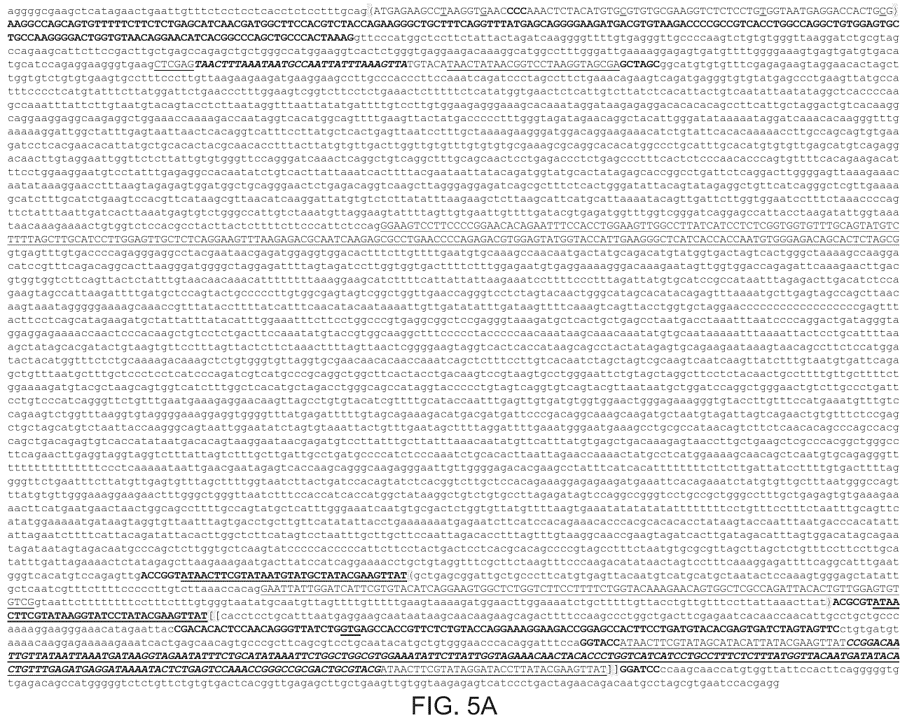

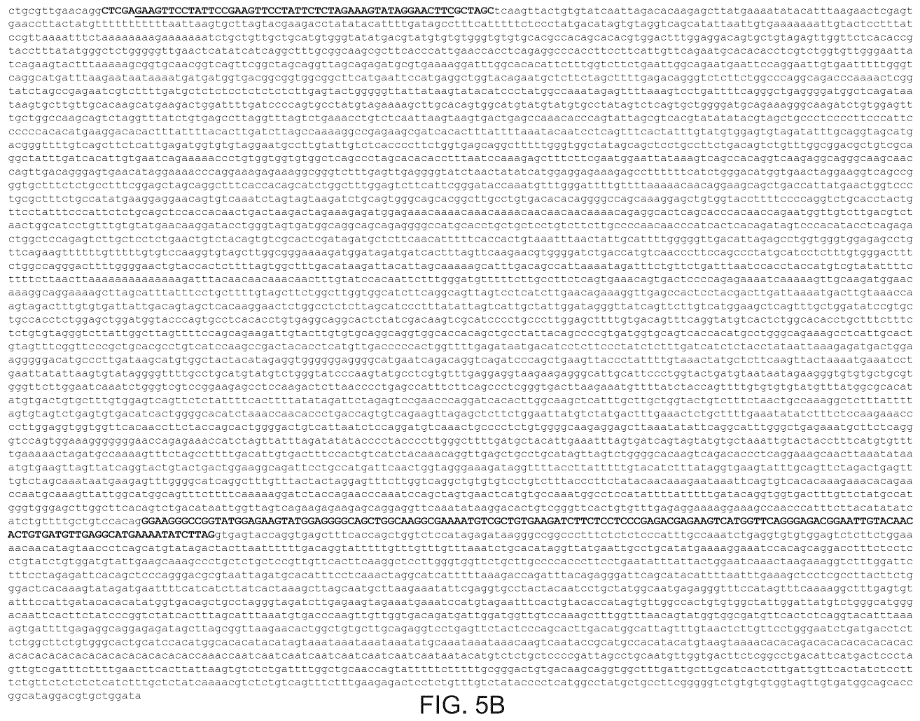

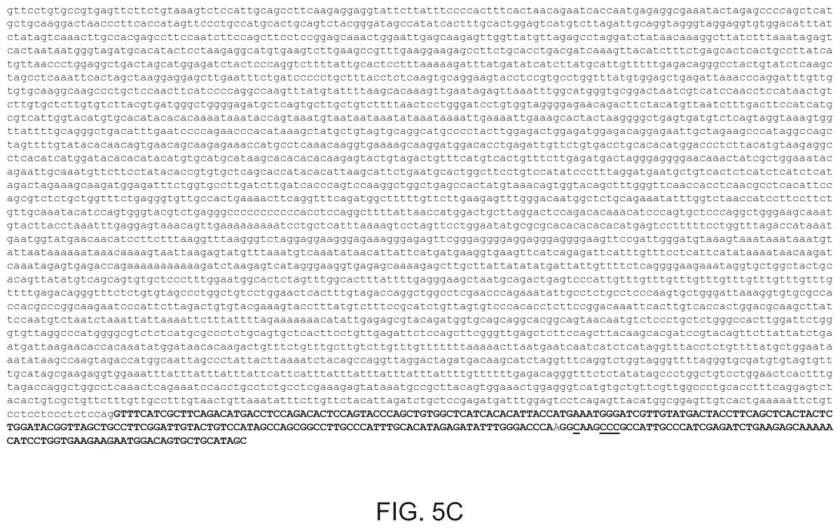

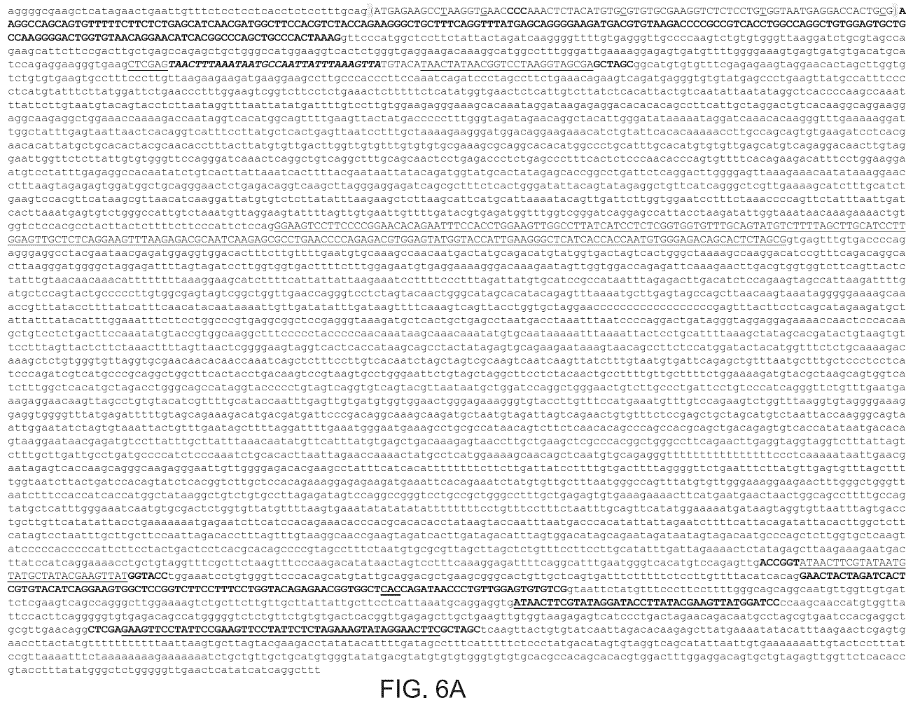

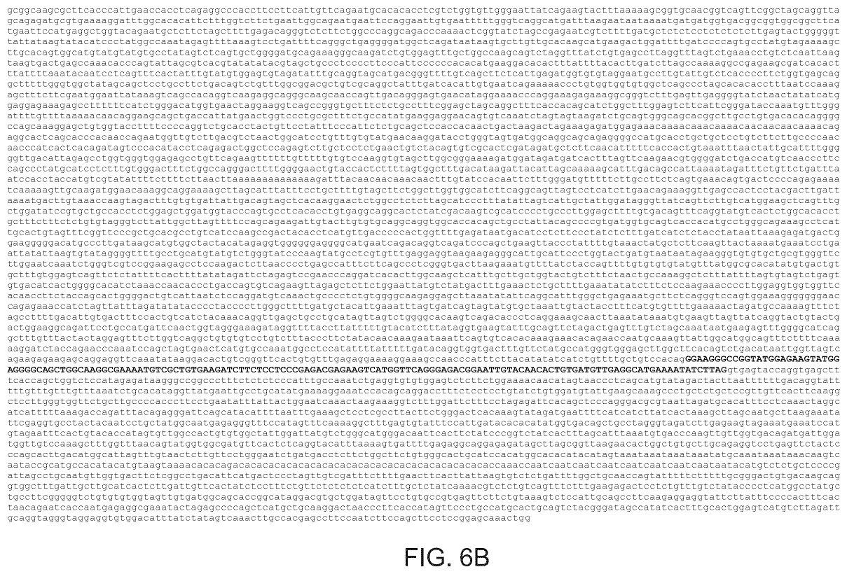

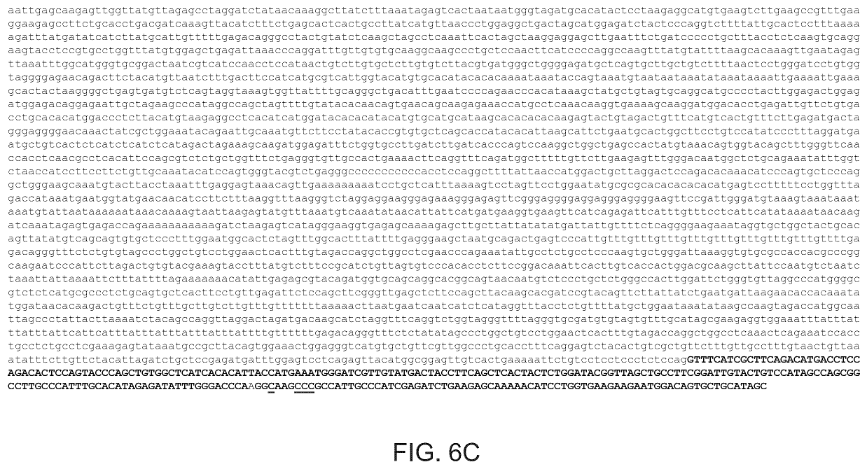

FIG. 1A depicts the genomic structure of a wild-type (unmodified) mouse Acvr1 locus. FIG. 1B illustrates a design of a targeting construct for modifying the endogenous mouse Acvr1 locus, resulting in an engineered Acvr1 allele designated as the “8431 allele”. Specifically, the nucleotide sequence of the first 78 bp of mouse Acvr1 coding exon 2 was replaced with the corresponding human ACVR1 sequence, with 6 silent mutations and Q (CAG) to P (CCC) coding for human amino acid 30. The rest of exon 2 remains mouse. A 45 bp deletion was made in mouse intron 2 for a loss-of-allele Taqman assay and to insert a Prm-Dre hUb-Hygro cassette. Following the cassette, the remainder of intron 2 was not changed. In intron 3, a loxP-3′ human ACVR1 intron 3-human coding exon 4-5′ human intron 4 (336 bp total)-lox2372 sequence was inserted. Following the lox2372 was a reverse oriented sequence consisting of mouse intron 4-mouse exon 4 with R206H FOP mutation-mouse intron 3 (328 bp total). This was followed by a loxP site in reverse orientation relative to the 5′ site, a rabbit HBB2 splice acceptor sequence in reverse orientation, and a lox2372 site, in reverse relative to its 5′ counterpart. This was followed by additional mouse intron 4 sequence (in forward orientation), then a Frt-hUb-Neo-Frt cassette. In mouse coding exon 6, a synonymous G to C point mutation was made, along with a S330P (TCC to CCC) humanizing change. FIG. 1C sets forth the constituents and sequences of the various fragments in the 8431allele identified as “A”, “B”, “C”, “D”, “E”, “F”, and “G” in FIG. 1B. FIG. 1D depicts the engineered Acvr1 allele after the Neo and Hygro resistance cassettes in the 8431 allele were deleted by FlpO and Dre recombinases, respectively. The resulting allele is designated as the 8432 allele.

The targeting construct for generating the 8431 allele was generated based on the following mouse and human sequences:

TABLE 2

| | NCBI | RefSeq | UniProt | Genomic | |
|---|---|---|---|---|---|
| | GeneID | mRNA ID | ID | Assembly | Location |
| Mouse | 11477 | NM_001355049.1 | P37172 | GRCm39 | Chr2: 58336450 . . . 58456840, - strand |
| Acvr1 | | (SEQ ID NO: 1) | (SEQ ID NO: 2) | | |
| Human | 90 | NM_001105.5 | Q04771 | GRCh38.p13 | Chr2: 157736446 . . . 157876330, - strand |
| ACVR1 | | (SEQ ID NO: 3) | (SEQ ID NO: 4) | | |

The targeting nucleic acid construct was electroporated into F1H4 mouse embryonic stem (ES) cells. Successful integration was confirmed by a modification of allele (MOA) assay as described, e.g., in Valenzuela et al., supra. Primers and probes used for the MOA assay are described in Tables 3-4, and their locations are shown in FIGS. 1A-1B. Neo and Hygro resistance cassettes were then deleted by FlpO and Dre recombinases, respectively.

TABLE 3
The below TaqMan assays are present in wild-type
alleles, absent in 8431, 8432, 8955 alleles.
7340mTD2

| | |
|---|---|
| Fwd | GCATCCAGAGGAAGGGTGAA (SEQ ID NO: 12) |
| Probe | ACTGCAGGTCATCAC (SEQ ID NO: 13) |
| Rev | ACATGCCCCATACACCCATT (SEQ ID NO: 14) |

Acvri5U

| | |
|---|---|
| Fwd | GGCTGACTGATCTGAAGGAAATGG (SEQ ID NO: 15) |
| Probe | TCTGGATAGTAAGGTCAGTTGCTGCG (SEQ ID |
| | NO: 16) |
| Rev | AGAGGAAGGAGACGCTAAGAATC (SEQ ID NO: 17) |

Acvri5D

| | |
|---|---|
| Fwd | TGAAGGAAATGGGCTTCTGGATAG (SEQ ID NO: 18) |
| Probe | AAGGTCAGTTGCTGCGTCTTCCC (SEQ ID NO: 19) |
| Rev | CATACTCACTCTTCCTGTTAGAGGA (SEQ ID |
| | NO: 20) |

8431mAS.WT

| | |
|---|---|
| Fwd | CCTTGCCCATTTGCACATAGA (SEQ ID NO: 21) |
| Probe | AAGGGAAGTCCGCCA (SEQ ID NO: 22) |
| Rev | TGTTTTTGCTCTTCAGATCTCGAT (SEQ ID NO: 23) |

TABLE 4
The below TaqMan assays are absent in wild-type
alleles, present in 8431, 8432, 8955 alleles
7340hTD

| | |
|---|---|
| Fwd | TTTGCAGATGAGAAGCCTAAGGT (SEQ ID |
| | NO: 24) |
| Probe | CAAACTCTACATGTGCGT (SEQ ID NO: 25) |
| Rev | CGCAGTGGTCCTCATTACCA (SEQ ID NO: 26) |

8431mAS.Mut

| | |
|---|---|
| Fwd | CCTTGCCCATTTGCACATAGA (SEQ ID NO: 27) |
| Probe | AAGGCAAGCCCGCCA (SEQ ID NO: 28) |
| Rev | TGTTTTTGCTCTTCAGATCTCGAT (SEQ ID |
| | NO: (29) |

Positively targeted ES cells were used as donor ES cells and microinjected into a pre-morula (8-cell) stage mouse embryo by the VELOCIMOUSE® method (see, e.g., U.S. Pat. Nos. 7,576,259, 7,659,442, 7,294,754, and US 2008-0078000 A1, all of which are incorporated herein by reference). The mouse embryo comprising the donor ES cells was incubated in vitro and then implanted into a surrogate mother to produce an F0 mouse fully derived from the donor ES cells. Mice bearing the engineered Acvr1 allele were identified by genotyping using the MOA assay described above. Mice heterozygous for the engineered Acvr1 allele were bred to homozygosity. The engineered Acvr1 alleles (8341 and 8342) are also referred herein as Acvr1^{huecto[R206H]FIEx;[S330P]/+}.

### Example 2. Phenotyping of Acvr1^{huecto[R206H]FIEx;[S330P]/+}; Nanog-Cre Mice

Experimental Procedures—Acvr1^{huecto[R206H]FIEx;[S330P]/+} males were mated with Nanog-Cre females (Tg(Nanog-cre)#Vlcg). The details of the Tg(Nanog-cre)#Vlcg mice are available at the Mouse Genome Informatics web site under MGI ID 5545911. Briefly, a transgene comprising a nucleic acid sequence encoding recombinase Cre, operably linked to a Nanog promoter, is inserted in the genome of this mouse strain. Resultant pups from the cross surviving to 23 days of age (n=83) were genotyped and assessed for gross morphological defects, heterotopic bone formation, and survival. Select Acvr1^{huecto[R206H]FIEx;[S330P]/+}; Nanog-Cre (n=15) and wild-type (n=4) mice were subjected to muscle pinch injury 14 days prior to the study end date. Heterotopic bone formation was assessed using in vivo μCT.

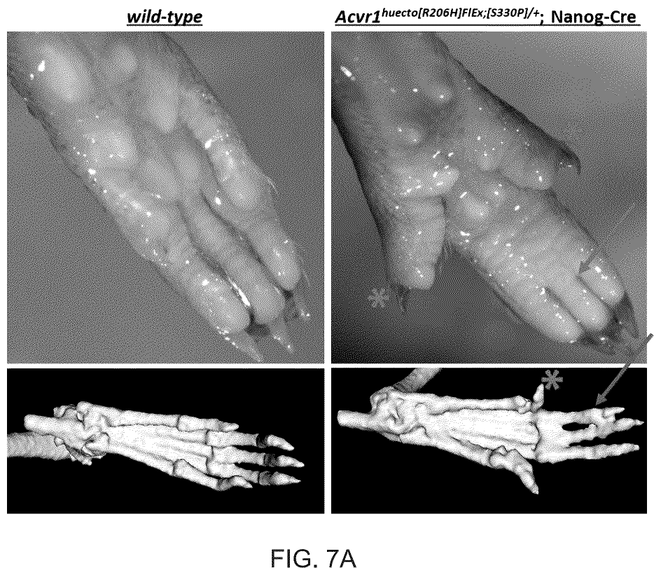

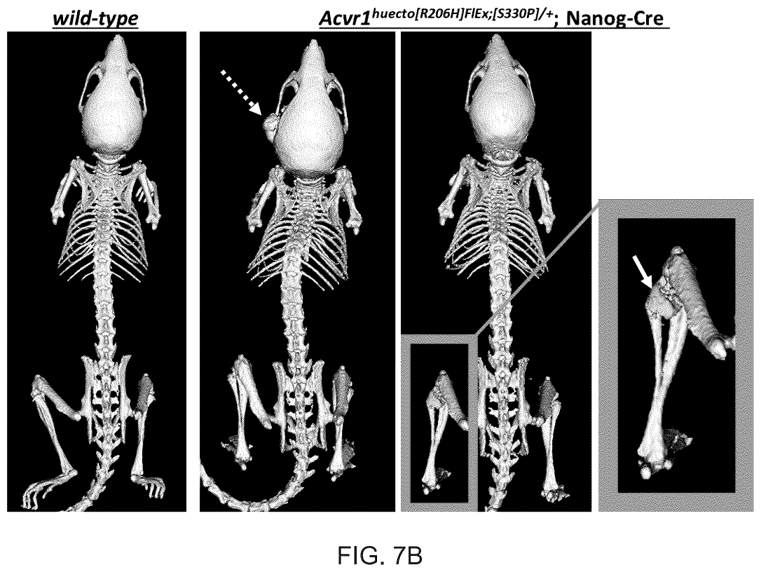

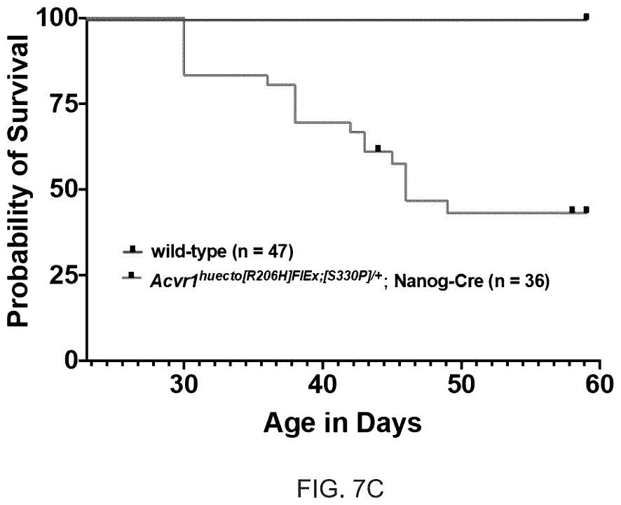

Results—Acvr1^{huecto[R206H]FIEx;[S330P]/+}; Nanog-Cre mice were born at mendelian ratios 36 of 83 pups genotyped as Acvr1^{huecto[R206H]FIEx;[S330P]/+}; Nanog-Cre at 23 days of age. With 100% penetrance, Acvr1^{huecto[R206H]FIEx;[S330P]/+}; Nanog-Cre mice exhibited persistent interdigital webbing between hindlimb digits 2-4 and truncation of hindlimb digits 1 and 5 (FIG. 7A). 15 of 37 Acvr1^{huecto[R206H]FIEx;[S330P]/+}; Nanog-Cre mice were imaged by in vivo μCT at 4-6 wks of age prior to hindlimb muscle pinch injury. 74% presented with one or more sights of overt heterotopic ossification (HO). Of these 40% had HO ankylosing the mandible, 30% had posterior knee region HO, and 13% had ankle region HO. See FIG. 7B. Acvr1^{huecto[R206H]FIEx;[S330P]/+}; Nanog-Cre mice exhibit median survival of 46 days post-birth, likely due to jaw ankylosing HO impairing feeding (FIG. 7C). 100% of Acvr1^{huecto[R206H]FIEx;[S330P]/+}; Nanog-Cre displayed HO 14 days following injury. HO was not detected in wild-type mice.

Discussion-Fibrodysplasia ossificans progressiva (FOP) is a particularly rare and exceedingly disabling genetic disease in which heterotopic ossification (HO) results in joint ankylosis and destruction of skeletal muscle and its associated soft tissues. In FOP, activating mutations in the type I bone morphogenetic protein (BMP) receptor ACVR1 render it neo-responsive to Activin A, which mediates induction and progression of HO. Although neo-responsiveness to Activin A is shared by all FOP-causing ACVR1 mutations, ˜95% of FOP patients possess Arginine 206 to a Histidine (R206H) substation the in the intracellular domain ACVR1. In juxtaposition to the devastatingly disabling consequences of HO during post-natal life, the developmental malformations associated with FOP are comparatively benign, the most overt of which being a truncating malformation of the great toe. However, despite mouse and human ACVR1 proteins sharing ˜98% sequence identity, Acvr1R206H/+ mice die perinatally. This initial observation led us to generate a ‘conditional-on’ mouse model of FOP: Acvr1^{huecto[R206H]FIEx;[S330P]/+}; this model has been used successfully to discover the key molecular and cellular mechanism that drives HO in FOP. Nonetheless, when the Acvr1^{huecto[R206H]FIEx;[S330P]/+} model is recombined early in development using Nanog-Cre, the resulting Acvr1^{R206H/+}; Nanog-Cre mice display both neonatal lethality and skeletal deformities that are substantially more severe than those observed in humans with FOP. As shown herein, humanizing mouse Acvr1[R206H] by substituting Serine 330 with a Proline, as is found in human ACVR1, alleviated neonatal lethality. Additionally, the resultant Acvr1^{huecto[R206H]FIEx;[S330P]/+}; Nanog-Cre mice exhibited congenital toe malformations and developed injury-induced and idiopathic HO in post-natal life, closely recapitulating human FOP. Studies using mouse embryonic stem cells indicate that the humanized ACVR1[R206H;S330P] receptor signals less potently than mouse ACVR1[R206H], which is consistent with the differential phenotypic severity observed in Acvr1R206H/+; Nanog-Cre and Acvr1^{huecto[R206H]FIEx;[S330P]/+}; Nanog-Cre mice. Hence, Acvr1^{huecto[R206H]FIEx;[S330P]/+} mice represent the most physiological relevant genetically engineered mouse model of FOP developed to date.

### Example 3. An Anti-Activin a Blocking Antibody Inhibits HO Formation and Promotes Survival in Acvr1^{[R206h, S330p]} Mice

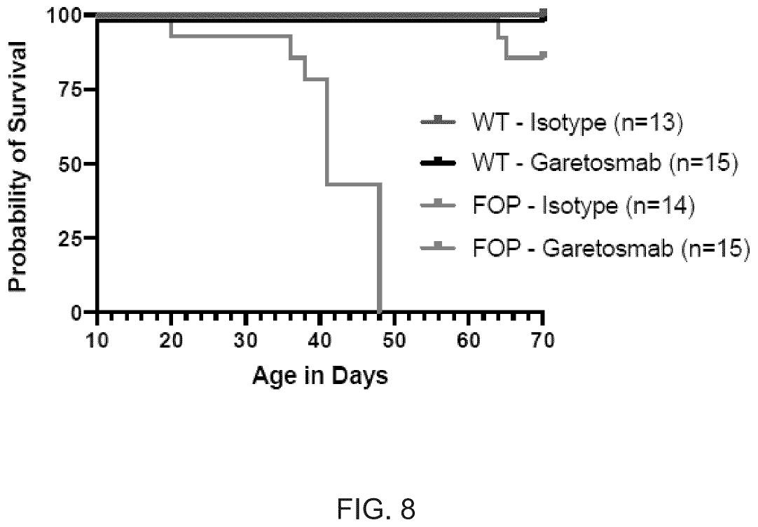

Wild type mice and Acvr1^{huecto[R206H]FIEx;[S330P]/+}; Nanog-Cre mice (“FOP mice”) were treated with an anti-Activin A monoclonal antibody (Garetosmab) and an isotype control antibody. When the Garetosmab treatment was initiated at 2 weeks of age, FOP mice exhibited 100% survival through 8 weeks of age (n=15). In contrast, FOP mice exhibit a median survival of ˜6 weeks in the absence of Garetosmab (n=14). The deaths in untreated FOP mice are believed to be possibly due to jaw HO. The results are shown in FIG. 8, demonstrating that the anti-Activin A antibody (Garetosmab) inhibited HO formation and promoted survival of the FOP mice.

### Example 4. S330P Mutation Makes ACVR1 Less Responsive to Ligand and Antibody Activation

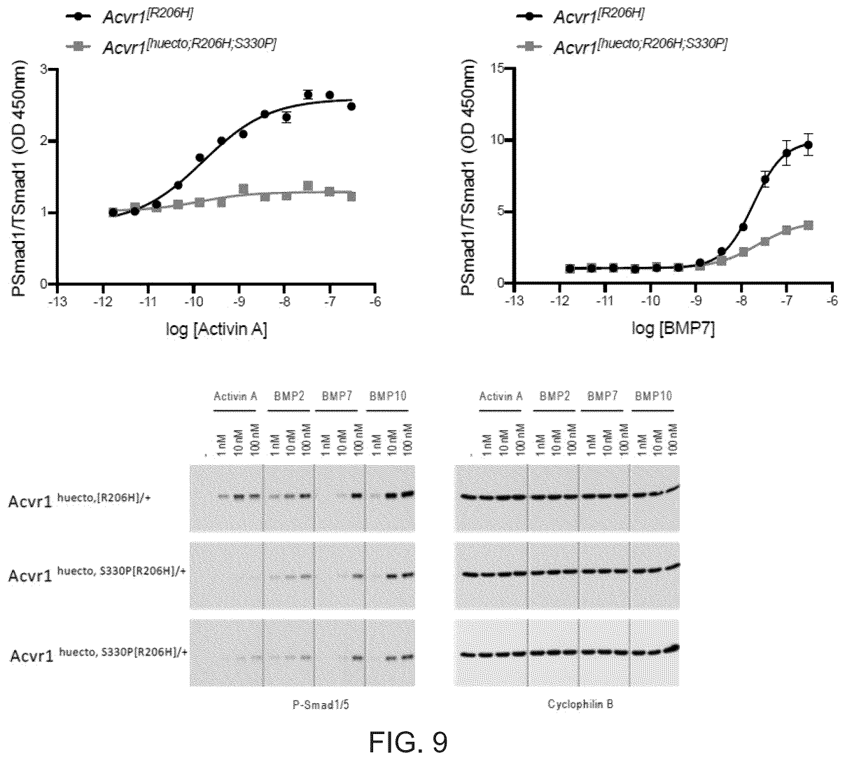

Acvr1^{[R206H]/+} and Acvr1^{huecto[R206H]FIEx;[S330P]/+} mES cells were treated with Activin A and BMP7 for 1 hr (after 1 hr starvation). In-cell ELISA was performed with cell lysates to measure P-Smad1 and Total Smad1 levels. The ratio of P-Smad1/T-Smad1 was calculated and plotted against the ligand concentration. Cell lysates were also run on the Western blots to compare the P-Smad1/5/8 levels of Acvr1^{[R206H]/+} and Acvr1^{huecto[R206H]FIEx;[S330P]/+} mES cells treated with Activin A, BMP2, BMP7, and BMP10 for 1 hr. Cyclophilin B was used as a loading control in the immunoblot. Both in-cell ELIS and immunoblot data (FIG. 9) show that the S330P mutation made ACVR1 less responsive to ligand activation, which is indicated by significantly decreased P-Smad1/T-Smad1 levels in ELIS assay and P-Smad1/5 levels in the Western blot of Acvr1^{huecto[R206H]FIEx;[S330P]/+} mES cells treated with ligands compared to the ones of Acvr1^{[R206H]/+} mES cells.

### Example 5. Mouse Acvr1 Kinase Domain is More Active than Human

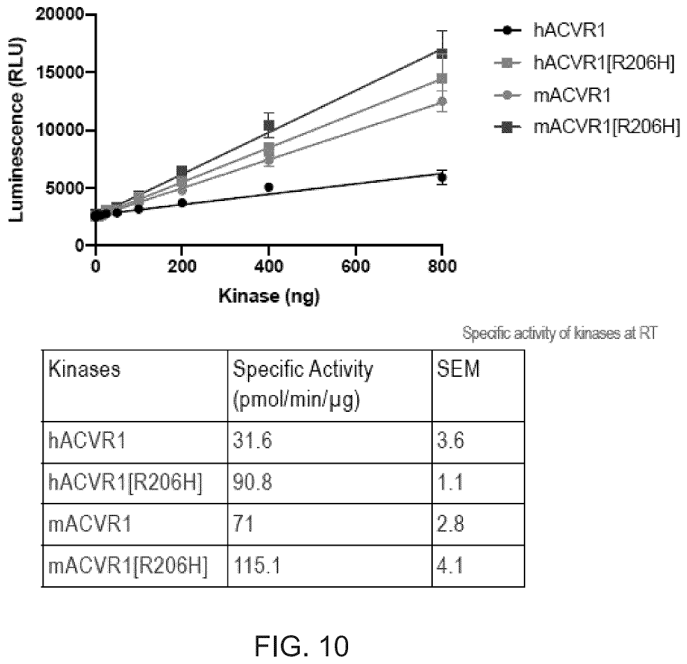

N-terminally His tagged human ACVR1 (“hACVR1”), hACVR1 [R206H], mouse ACVR1 (“mACVR1”), and mACVR1 [R206H] were expressed in ExpiCHO cells and purified (Ni-column followed by size exclusion chromatography-SEC). The kinase activity (ability to phosphorylate casein as a substrate) of the purified human and mouse ACVR1 kinases was compared at room temperature (“RT”). In this experiment, a fixed amount of casein substrate and a fixed amount of ATP were incubated with different amounts of purified human and mouse ACVR1 kinases. After unused ATP was depleted, the produced ADP was re-converted to ATP, which was converted into light by luciferase. The luciferase light generated was correlated with the amount of ADP generated in the kinase assay, which is indicative of kinase activity. The data, as shown in FIG. 10, demonstrate that mACVR1 kinase is significantly more active than hACVR1 kinase.

What is claimed is:

1. A genetically modified rodent, comprising a modified rodent Acvr1 gene at an endogenous rodent Acvr1 locus encoding a modified rodent Acvr1 polypeptide,
wherein the modified rodent Acvr1 polypeptide comprises the ectodomain of a human ACVR1 protein, and the transmembrane and cytoplasmic domains of an endogenous rodent Acvr1 protein except for a S330P substitution and an FOP mutation selected from a R206H substitution or a R258G substitution; and
wherein expression of the modified rodent Acvr1 gene is under control of the rodent Acvr1 promoter at the endogenous rodent Acvr1 locus.

2. The rodent claim 1, wherein exon 2 of the modified rodent Acvr1 gene differs from exon 2 of an endogenous rodent Acvr1 gene by comprising
(i) a substitution of the codon for Q at position 30 with a codon for P, or
(ii) a replacement of a sequence in exon 2 of the endogenous rodent Acvr1 gene encoding endogenous rodent Acvr1 ectodomain amino acids including Q30, with either a 5′ sequence of a human ACVR1 exon 2 encoding human ACVR1 ectodomain amino acids comprising P at position 30, or a sequence modified from the 5′ sequence of the human ACVR1 exon 2 to include one or more silent mutations.

3. The rodent of claim 2, wherein said human ACVR1 ectodomain amino acids comprise amino acids from position 24 to position 49.

4. The rodent according to any one of claims 1-3, wherein exon 6 of the modified rodent Acvr1 gene differs from exon 6 of the endogenous rodent Acvr1 gene by comprising a substitution of the codon for Ser at position 330 with a codon for Pro, optionally by further comprising a synonymous nucleotide substitution.

5. The rodent according to any one of claims 1-4, wherein exon 4 of the modified rodent Acvr1 gene differs from exon 4 of the endogenous rodent Acvr1 gene by comprising a substitution of the codon for R at position 206 with a codon for H, optionally by further comprising a replacement of a sequence of the endogenous rodent Acvr1 exon 4 with a corresponding sequence of human ACVR1 exon 4 wherein the replacement does not change the amino acids encoded by the endogenous rodent Acvr1 exon 4.

6. The rodent according to any one of claims 1-5, wherein the modified rodent Acvr1 gene comprises:
an endogenous rodent Acvr1 exon 1,
a modified rodent Acvr1 exon 2 which differs from an endogenous rodent Acvr1 exon 2 by comprising a substitution of the codon for Q30 with a codon for P;
an endogenous rodent Acvr1 exon 3;
a modified rodent Acvr1 exon 4 which differs from an endogenous rodent Acvr1 exon 4 by comprising a substitution of the codon for R206 with a codon for H;
an endogenous rodent Acvr1 exon 5;
a modified rodent Acvr1 exon 6 that differs from exon 6 of the endogenous rodent Acvr1 gene by comprising a substitution of the codon for S330 with a codon for P; and
endogenous rodent Acvr1 exons 7-9.

7. The rodent of claim 6, wherein
the modified rodent Acvr1 exon 2 differs from the endogenous rodent Acvr1 exon 2 by comprising a replacement of a 5′ sequence of the endogenous rodent Acvr1 exon 2 with (i) a 5′ sequence of a human ACVR1 exon 2 wherein the 5′ sequence of the human ACVR1 exon 2 encodes human ACVR1 amino acids comprising P at position 30, or (ii) a sequence modified from the 5′ sequence of the human ACVR1 exon 2 to include one or more silent mutations; and/or
the modified rodent Acvr1 exon 4 encoding R206H differs from the endogenous rodent Acvr1 exon 4 by comprising a replacement of a sequence of the endogenous rodent Acvr1 exon 4 with a sequence of human ACVR1 exon 4 and a substitution of the codon for R at position 206 with a codon for H; and/or
the modified rodent Acvr1 exon 6 differs from the endogenous rodent Acvr1 exon 6 by comprising a substitution of the codon for S at position 330 with a codon for P and a synonymous nucleotide substitution.

8. The rodent according to any one of claims 1-5, wherein the modified rodent Acvr1 gene comprises:
an endogenous rodent Acvr1 exon 1,
a modified rodent Acvr1 exon 2 which differs from an endogenous rodent Acvr1 exon 2 by comprising a substitution of the codon for Q30 with a codon for P;
an endogenous rodent Acvr1 exon 3;
an endogenous rodent Acvr1 exon 4;
a modified rodent Acvr1 exon 5 which differs from an endogenous rodent Acvr1 exon 5 by comprising a substitution of the codon for R258 with a codon for G;
a modified rodent Acvr1 exon 6 that differs from an endogenous rodent Acvr1 exon 6 by comprising a substitution of the codon for S330 with a codon for P; and
endogenous rodent Acvr1 exons 7-9.

9. The rodent of claim 8, wherein:
the modified rodent Acvr1 exon 2 differs from the endogenous rodent Acvr1 exon 2 by comprising a replacement of a 5′ sequence of the endogenous rodent Acvr1 exon 2 with (i) a 5′ sequence of a human ACVR1 exon 2 wherein the 5′ sequence of the human ACVR1 exon 2 encodes human ACVR1 amino acids comprising P at position 30, or (ii) a sequence modified from the 5′ sequence of the human ACVR1 exon 2 to include one or more silent mutations; and/or
the modified rodent Acvr1 exon 6 differs from the endogenous rodent Acvr1 exon 6 by comprising a substitution of the codon for S at position 330 with a codon for P and a synonymous nucleotide substitution.

10. The rodent of according to any one of claims 1-9, wherein the modified rodent Acvr1 gene is in the germline genome of the rodent.

11. The rodent of according to any one of claims 1-9, wherein the modified rodent Acvr1 gene is formed at an embryonic stage from an engineered Acvr1 gene in the rodent genome,
wherein the engineered Acvr1 gene encodes an engineered Acvr1 polypeptide comprising the ectodomain of a human ACVR1 protein, and the transmembrane and cytoplasmic domains of the endogenous rodent Acvr1 protein except for the S330P substitution; and
wherein the engineered Acvr1 gene comprises:
a. a human ACVR1 exon 4 in sense orientation flanked by a first pair of site-specific recombinase recognition sites (SRRS′), and a mutant rodent Acvr1 exon 4 encoding R206H in antisense orientation, flanked by a second pair of SRRS' that are different from the first pair of SRRS′, wherein the first and second pairs of SRRS' are oriented so that a recombinase can invert the mutant rodent Acvr1 exon 4 into sense orientation and delete the human ACVR1 exon 4 to form said modified rodent Acvr1 gene; or
b. a human ACVR1 exon 5 in sense orientation flanked by a first pair of site-specific recombinase recognition sites (SRRS′), and a mutant rodent Acvr1 exon 5 encoding R258G in antisense orientation, flanked by a second pair of SRRS' that are different from the first pair of SRRS′, wherein the first and second pairs of SRRS' are oriented so that a recombinase can invert the mutant rodent Acvr1 exon 5 into sense orientation and delete the human ACVR1 exon 5 to form said modified rodent Acvr1 gene.

12. The rodent of claim 11, wherein the recombinase is Cre.

13. The rodent of claim 11 or 12, wherein the genome of the rodent comprises a polynucleotide encoding the recombinase under control of a Nanog promoter.

14. The rodent according to any one of claims 1-13, wherein the rodent is heterozygous for the modified Acvr1 gene.

15. The rodent according to any of the preceding claims, selected from a mouse or a rat.

16. The rodent according to any of the preceding claims, which survives at least 2-3 weeks after birth, exhibits congenital toe malformations and develop injury-induced and idiopathic HO in post-natal life.

17. An isolated tissue or cell of the rodent according to any of the preceding claims, wherein the isolated tissue or cell comprises the modified rodent Acvr1 gene.

18. A rodent embryonic stem (ES) cell, comprising a modified rodent Acvr1 gene at an endogenous rodent Acvr1 locus encoding a modified rodent Acvr1 polypeptide,
wherein the modified rodent Acvr1 polypeptide comprises the ectodomain of a human ACVR1 protein, and the transmembrane and cytoplasmic domains of an endogenous rodent Acvr1 protein except for a S330P substitution and an FOP mutation selected from a R206H mutation or a R258G mutation; and
wherein expression of the modified rodent Acvr1 gene is under control of the rodent Acvr1 promoter at the endogenous rodent Acvr1 locus.

19. A rodent embryonic stem (ES) cell, comprising an engineered rodent Acvr1 gene at an endogenous rodent Acvr1 locus,
wherein the engineered Acvr1 gene encodes an engineered Acvr1 polypeptide comprising the ectodomain of a human ACVR1 protein, and the transmembrane and cytoplasmic domains of the endogenous rodent Acvr1 protein except for a S330P substitution; and
wherein the engineered Acvr1 gene comprises:
(i) a human ACVR1 exon 4 in sense orientation flanked by a first pair of site-specific recombinase recognition sites (SRRS′), and a mutant rodent Acvr1 exon 4 encoding R206H in antisense orientation, flanked by a second pair of SRRS' that are different from the first pair of SRRS′, wherein the first and second pairs of SRRS' are oriented so that a recombinase can invert the mutant rodent Acvr1 exon 4 into sense orientation and delete the human ACVR1 exon 4 to form a modified rodent Acvr1 gene; or
(ii) a human ACVR1 exon 5 in sense orientation flanked by a first pair of site-specific recombinase recognition sites (SRRS′), and a mutant rodent Acvr1 exon 5 encoding R258G in antisense orientation, flanked by a second pair of SRRS' that are different from the first pair of SRRS′, wherein the first and second pairs of SRRS' are oriented so that a recombinase can invert the mutant rodent Acvr1 exon 5 into sense orientation and delete the human ACVR1 exon 5 to form a modified rodent Acvr1 gene.

20. The rodent ES cell of claim 18 or 19, wherein the rodent is mouse or rat.

21. A rodent embryo comprising the rodent ES cell of any one of claims 18-20.

22. A nucleic acid construct, comprising a modified rodent Acvr1 gene sequence, flanked by a 5′ homology arm and a 3′ homology arm, wherein the modified rodent Acvr1 gene sequence comprises:
a modified rodent Acvr1 exon 2 which differs from a wild-type rodent Acvr1 exon 2 by comprising a substitution of the codon for Q30 with a codon for P;
a wild-type rodent Acvr1 exon 3;
a modified rodent Acvr1 exon 4 which differs from a wild-type rodent Acvr1 exon 4 by comprising a substitution of the codon for R206 with a codon for H;
a wild-type rodent Acvr1 exon 5; and
a modified rodent Acvr1 exon 6 which differs from a wild-type rodent Acvr1 exon 6 by comprising a substitution of the codon for S330 with a codon for P;
wherein the 5′ homology arm and the 3′ homology arm are substantially identical to the sequences at a rodent Acvr1 gene locus to mediate integration of the modified rodent Acvr1 gene sequence into the rodent Acvr1 gene.

23. A nucleic acid construct, comprising a modified rodent Acvr1 gene sequence, flanked by a 5′ homology arm and a 3′ homology arm, wherein the modified rodent Acvr1 gene sequence comprises:
a modified rodent Acvr1 exon 2 which differs from a wild type rodent Acvr1 exon 2 by comprising a substitution of the codon for Q30 with a codon for P;
a wild type rodent Acvr1 exon 3;
a wild type rodent Acvr1 exon 4;
a modified rodent Acvr1 exon 5 which differs from a wild type rodent Acvr1 exon 5 by comprising a substitution of the codon for R258 with a codon for G;
a modified rodent Acvr1 exon 6 that differs from a wild-type rodent Acvr1 exon 6 by comprising a substitution of the codon for S330 with a codon for P; and
wherein the 5′ homology arm and the 3′ homology arm are substantially identical to the sequences at a rodent Acvr1 gene locus to mediate integration of the modified rodent Acvr1 gene sequence into the rodent Acvr1 gene.

24. A nucleic acid construct, comprising an engineered rodent Acvr1 gene sequence, flanked by a 5′ homology arm and a 3′ homology arm, wherein the engineered rodent Acvr1 gene sequence comprises:
a modified rodent Acvr1 exon 2 which differs from a wild-type rodent Acvr1 exon 2 by comprising a substitution of the codon for Q30 with a codon for P;
a wild-type rodent Acvr1 exon 3;
a human ACVR1 exon 4 in sense orientation flanked by a first pair of site-specific recombinase recognition sites (SRRS′), and a mutant rodent Acvr1 exon 4 encoding R206H in antisense orientation, flanked by a second pair of SRRS' that are different from the first pair of SRRS′, wherein the first and second pairs of SRRS' are oriented so that a recombinase can invert the mutant rodent Acvr1 exon 4 into sense orientation and delete the human ACVR1 exon 4;
a wild-type rodent Acvr1 exon 5; and
a modified rodent Acvr1 exon 6 that differs from a wild-type rodent Acvr1 exon 6 by comprising a substitution of the codon for S330 with a codon for P;
wherein the 5′ homology arm and the 3′ homology arm are substantially identical to the sequences at a rodent Acvr1 gene locus to mediate integration of the engineered rodent Acvr1 gene sequence into the rodent Acvr1 gene.

25. A nucleic acid construct, comprising an engineered rodent Acvr1 gene sequence, flanked by a 5′ homology arm and a 3′ homology arm, wherein the engineered rodent Acvr1 gene sequence comprises:
a modified rodent Acvr1 exon 2 which differs from a wild type rodent Acvr1 exon 2 by comprising a substitution of the codon for Q30 with a codon for P;
a wild type rodent Acvr1 exon 3;
a wild type rodent Acvr1 exon 4;
a human ACVR1 exon 5 in sense orientation flanked by a first pair of site-specific recombinase recognition sites (SRRS′), and a mutant rodent Acvr1 exon 5 encoding R258G in antisense orientation, flanked by a second pair of SRRS' that are different from the first pair of SRRS′, wherein the first and second pairs of SRRS' are oriented so that a recombinase can invert the mutant rodent Acvr1 exon 5 into sense orientation and delete the human ACVR1 exon 5;
a modified rodent Acvr1 exon 6 that differs from a wild-type rodent Acvr1 exon 6 by comprising a substitution of the codon for S330 with a codon for P; and
wherein the 5′ homology arm and the 3′ homology arm are substantially identical to the sequences at a rodent Acvr1 gene locus to mediate integration of the engineered rodent Acvr1 gene sequence into the rodent Acvr1 gene.

26. A method of making a genetically modified rodent, comprising modifying the rodent genome to comprise a modified rodent Acvr1 gene at an endogenous rodent Acvr1 locus,
wherein the modified rodent Acvr1 gene encodes a modified rodent Acvr1 polypeptide,
wherein the modified rodent Acvr1 polypeptide comprises the ectodomain of a human ACVR1 protein, and the transmembrane and cytoplasmic domains of an endogenous rodent Acvr1 protein except for a S330P substitution and an FOP mutation selected from a R206H mutation or a R258G mutation; and
wherein expression of the modified rodent Acvr1 gene is under control of the rodent Acvr1 promoter at the endogenous rodent Acvr1 locus.

27. The method of claim 26, wherein said modifying comprises modifying the genome of a rodent ES cell to comprise said modified rodent Acvr1 gene at the endogenous rodent Acvr1 locus, thereby obtaining a genetically modified rodent ES cell, and generating a rodent from the obtained genetically modified rodent ES cell.

28. The method of claim 27, wherein the genome of the rodent ES cell is modified by introducing a nucleic acid construct according to claim 22 or 23.

29. A method of making a genetically modified rodent, comprising modifying a rodent genome to comprise an engineered rodent Acvr1 gene at an endogenous rodent Acvr1 locus,
wherein the engineered Acvr1 gene encodes an engineered Acvr1 polypeptide comprising the ectodomain of a human ACVR1 protein, and the transmembrane and cytoplasmic domains of the endogenous rodent Acvr1 protein except for the S330P substitution; and
wherein the engineered Acvr1 gene comprises:
(a) a human ACVR1 exon 4 in sense orientation flanked by a first pair of site-specific recombinase recognition sites (SRRS′), and a mutant rodent Acvr1 exon 4 encoding R206H in antisense orientation, flanked by a second pair of SRRS' that are different from the first pair of SRRS′, wherein the first and second pairs of SRRS' are oriented so that a recombinase can invert the mutant rodent Acvr1 exon 4 into sense orientation and delete the human ACVR1 exon 4 to form a modified rodent Acvr1 gene; or
(b) a human ACVR1 exon 5 in sense orientation flanked by a first pair of site-specific recombinase recognition sites (SRRS′), and a mutant rodent Acvr1 exon 5 encoding R258G in antisense orientation, flanked by a second pair of SRRS' that are different from the first pair of SRRS′, wherein the first and second pairs of SRRS' are oriented so that a recombinase can invert the mutant rodent Acvr1 exon 5 into sense orientation and delete the human ACVR1 exon 5 to form a modified rodent Acvr1 gene.

30. The method of claim 29, wherein said modifying comprises modifying the genome of a rodent ES cell to comprise said engineered rodent Acvr1 gene at the endogenous rodent Acvr1 locus of the rodent ES cell, thereby obtaining a genetically modified ES cell, and generating a rodent from the obtained genetically modified ES cell.

31. The method of claim 30, wherein the genome of the rodent ES cell is modified by introducing a nucleic acid construct according to claim 24 or 25.

32. The method according to any one of claims 29-31, wherein the recombinase is Cre.

33. The method according to any one of claims 29-32, wherein the genome of the rodent comprises a polynucleotide encoding the recombinase under control of a Nanog promoter, and wherein the recombinase acts at an embryonic stage of the rodent to invert the mutant rodent Acvr1 exon into sense orientation and delete the wild-type Acvr1 exon thereby forming a modified rodent Acvr1 gene encoding a modified Acvr1 polypeptide comprising the ectodomain of a human ACVR1 protein, and the transmembrane and cytoplasmic domains of the endogenous rodent Acvr1 protein except for the S330P substitution and the FOP mutation.

34. The method according to any one of claims 26-33, wherein the rodent is a mouse or a rat.

35. A method of testing a candidate therapeutic compound for treating ectopic bone formation, comprising:
providing a genetically modified rodent according to any one of claims 1-16;
administering the candidate compound to the rodent; and
determining whether the candidate compound inhibits the development of ectopic bone formation in the rodent.
